SEQUENCE LISTING (362 sequences)

SEQ ID NO: 1 - DNA, 600 bp, Saccharomyces cerevisiae
  source 1..600: mol_type = unassigned DNA
gcgtattaccttctgctggattcaaacactcttctccagtaaaaagattcccttctcactctgcttagagaaggagtgccaggccggctaagcccactctccagacggaaaccatacaatgcctccgctggctgcattgtcgcgccccgcccaacgacggtttaccgacagctgctagctgggctcaacaggtggttagcccaccaattcccctgtcgctcttcgctctgaatgtgacggcaaatttcgacccgttgttcctgttccttttttttttcaattggactgaaaaaaaaaaagaaccgaatctggaaagatacacccaaacatacatagaatgtacggatgcatgattgtctcagcctcgtttggctcatcgttcttcatttctttttcctaattttgatagagacaatagatagacgtggaaggaaaaaaaaaaggaaagcccaacaatattgagaaacgaagaggtgtatttggtttaaatagagcctcttcattcctttcctgatctgacaacagggtggaacataaaatatagatctgtagtgagtgcgaatagcaatagtaagtgaacgaaaaaggaatacgataata

SEQ ID NO: 2 - DNA, 1491 bp, synthetic construct
  misc_feature 1..1491: note = Yeast optimized cas-alpha 10 gene
  source 1..1491: mol_type = other DNA
atgggagaatccgtgaaggccatcaaactgaagatcctggacatgttcctggacccagagtgtaccaaacaggacgacaactggagaaaggacctgagtaccatgtccaggttctgcggtgaagccggcaacatgtgtttgagggacctatacaactacttctccatgccaaaggaggaccgtatctcttccaaagacctatacaacgccatgtaccacaaaaccaagctgctgcacccaggtctgcccggcaaagttaaaaaccaaatcgtcaaccacgccaaggacgtctggaaaaggaacgccaagctgatatacaggaaccagatctccatgccaacatacaagatcaccaccgcccccatcaggctgcagaacaacatctacaagctgatcaagaacaagaacaagtacataatcgacgtccagctgtacagtaaggagtactcaaaggacagtggcaaaggcacccataggtacttcctggtcgcagtcagagactcatccaccaggatgatcttcgacaggataatgtccaaggatcacatcgacagttccaagtcctacaaacagggacagctgcagatcaagaaggaccaccagggcaagtggtactgcatcatcccctacaccttcccaactcatgagcatgtgttagaccccgacaaggtgatgggagtggacctgggcgtcgctaaagccgtctactgggccttcaacagttcctacaagaggggctgcatcgacggaggcgaaatcgagcatttccgcaagatgatcagggccaggagggtcagtatccagaaccagatcaaacacagtggagacgcccgtaagggccacggaaggaagcgtgctttgaagccaatcgagaccctgtctgagaaggaatcaaacttcagggacaccatcaactttaggtacgccaacaggatcgtcgaagccgccatcaagcagggctgcggaaccatccagatcgagaacctggaaggaatcgctgatagaaccggctccaagttcctgaagaactggccatactacgacctgcagaccaagatcgtcaacaaggccaaggagcacggaatcaccgtggttgccatcaacccacaatatacctcccagaggtgctccatgtgcggctacatcgagaagacaaacagatcctcccaggctgtcttcgaatgcaagcagtgcggctacggttccaggaccaattgcatcaactgcagacacgtccaagtttccggtgacgtctgcgaagagtgcggcggtatcgtcaaaaaggagaacgtgaacgcagactacaacgccgccaagaatatcagtaccccctacatcgaccagataatcatggagaaatgcctggagctaggcatcccctacaggtccatcacatgcaaggagtgtggccacatccaagccagtggcaatacctgcgaagtctgcggcagtaccaatatcctgaaacccaagaagatcaggaaggccaag

SEQ ID NO: 3 - DNA, 21 bp, synthetic construct
  misc_feature 1..21: note = Sequence encoding SV40 NLS
  source 1..21: mol_type = other DNA
ccaaagaagaagagaaaggtt

SEQ ID NO: 4 - DNA, 248 bp, Saccharomyces cerevisiae
  source 1..248: mol_type = unassigned DNA
tcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgc

SEQ ID NO: 5 - DNA, 270 bp, Saccharomyces cerevisiae
  source 1..270: mol_type = unassigned DNA
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcg

SEQ ID NO: 6 - DNA, 189 bp, synthetic construct
  misc_feature 1..189: note = Sequence encoding Cas-alpha 10 sgRNA (Cas-alpha recognitiondomain)
  source 1..189: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaac

SEQ ID NO: 7 - DNA, 20 bp, Saccharomyces cerevisiae
  source 1..20: mol_type = unassigned DNA
tttagtgtaggaacatcaac

SEQ ID NO: 8 - DNA, 20 bp, Saccharomyces cerevisiae
  source 1..20: mol_type = unassigned DNA
atcttcatcacctatgccat

SEQ ID NO: 9 - DNA, 209 bp, synthetic construct
  misc_feature 1..209: note = Sequence encoding Cas-alpha 10 sgRNA targeting ADE2
  source 1..209: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 10 - DNA, 209 bp, synthetic construct
  misc_feature 1..209: note = Sequence encoding Cas-alpha 10 sgRNA targeting CAN1
  source 1..209: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacatcttcatcacctatgccat

SEQ ID NO: 11 - DNA, 68 bp, Hepatitis delta virus
  source 1..68: mol_type = unassigned DNA
ggccggcatggtcccagcctcctcgctggcgccggctgggcaacatgcttcggcatggcgaatgggac

SEQ ID NO: 12 - DNA, 20 bp, Saccharomyces cerevisiae
  source 1..20: mol_type = unassigned DNA
tttttttgttttttatgtct

SEQ ID NO: 13 - protein, 7 aa, Betapolyomavirus macacae
PKKKRKV

SEQ ID NO: 14 - protein, 497 aa, synthetic construct
  REGION 1..497: note = Cas-alpha 10 endonuclease
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 15 - protein, 497 aa, synthetic construct
  REGION 1..497: note = Cas-alpha 10 T34E variant
MGESVKAIKLKILDMFLDPECTKQDDNWRKDLSEMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 16 - protein, 497 aa, synthetic construct
  REGION 1..497: note = Cas-alpha 10 S36A variant
MGESVKAIKLKILDMFLDPECTKQDDNWRKDLSTMARFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 17 - protein, 497 aa, synthetic construct
  REGION 1..497: note = Cas-alpha 10 M45S variant
MGESVKAIKLKILDMFLDPECTKQDDNWRKDLSTMSRFCGEAGNSCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 18 - protein, 497 aa, synthetic construct
  REGION 1..497: note = Cas-alpha 10 F54D variant
MGESVKAIKLKILDMFLDPECTKQDDNWRKDLSTMSRFCGEAGNMCLRDLYNYDSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 19 - protein, 497 aa, synthetic construct
  REGION 1..497: note = Cas-alpha 10 L77S variant
MGESVKAIKLKILDMFLDPECTKQDDNWRKDLSTMSRFCGEAGNMCLRDLYNYFSMPKEDRISSKDLYNAMYHKTKSLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 20 - protein, 497 aa, synthetic construct
  REGION 1..497: note = Cas-alpha 10 K85Q variant
MGESVKAIKLKILDMFLDPECTKQDDNWRKDLSTMSRFCGEAGNMCLRDLYNYFSMPKEDRISSKDLYNAMYHKTKLLHPGLPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 21 - protein, 497 aa, synthetic construct
  REGION 1..497: note = Cas-alpha 10 K155N variant
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGNGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 22 - protein, 497 aa, synthetic construct
  REGION 1..497: note = Cas-alpha 10 K198C variant
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKCDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 23 - protein, 497 aa, synthetic construct
  REGION 1..497: note = Cas-alpha 10 H215T variant
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTTEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 24 - protein, 497 aa, synthetic construct
  REGION 1..497: note = Cas-alpha 10 G320E variant
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQECGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 25 - protein, 497 aa, synthetic construct
  REGION 1..497: note = Cas-alpha 10 L455A variant
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCAELGIPYRSITCKECGHIQASGNTCEVCGSTNILKPKKIRKAK

SEQ ID NO: 26 - protein, 497 aa, synthetic construct
  REGION 1..497: note = Cas-alpha 10 I464T variant
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSTTCKECGHIQASGNTCEVCGSTNILKPKKIRKAK

SEQ ID NO: 27 - protein, 497 aa, synthetic construct
  REGION 1..497: note = Cas-alpha 10 T465E variant
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSIECKECGHIQASGNTCEVCGSTNILKPKKIRKAK

SEQ ID NO: 28 - protein, 497 aa, synthetic construct
  REGION 1..497: note = Cas-alpha 10 G470D variant
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

SEQ ID NO: 29 - protein, 497 aa, synthetic construct
  REGION 1..497: note = Cas-alpha 10 L488E variant
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNIEKPKKIRKAK

SEQ ID NO: 30 - protein, 497 aa, synthetic construct
  REGION 1..497: note = Cas-alpha 10 A496N variant
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKNK

SEQ ID NO: 31 - protein, 497 aa, synthetic construct
  REGION 1..497: note = Cas-alpha 10 F54D+T34E variant
MGESVKAIKLKILDMFLDPECTKQDDNWRKDLSEMSRFCGEAGNMCLRDLYNYDSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 32 - protein, 497 aa, synthetic construct
  REGION 1..497: note = Cas-alpha 10 F54D+S36A variant
MGESVKAIKLKILDMFLDPECTKQDDNWRKDLSTMARFCGEAGNMCLRDLYNYDSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 33 - protein, 497 aa, synthetic construct
  REGION 1..497: note = Cas-alpha 10 F54D+M45S variant
MGESVKAIKLKILDMFLDPECTKQDDNWRKDLSTMSRFCGEAGNSCLRDLYNYDSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 34 - protein, 497 aa, synthetic construct
  REGION 1..497: note = Cas-alpha 10 F54D+K85Q variant
MGESVKAIKLKILDMFLDPECTKQDDNWRKDLSTMSRFCGEAGNMCLRDLYNYDSMPKEDRISSKDLYNAMYHKTKLLHPGLPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 35 - protein, 497 aa, synthetic construct
  REGION 1..497: note = Cas-alpha 10 F54D+G470D variant
MGESVKAIKLKILDMFLDPECTKQDDNWRKDLSTMSRFCGEAGNMCLRDLYNYDSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

SEQ ID NO: 36 - protein, 497 aa, synthetic construct
  REGION 1..497: note = Cas-alpha 10 F54D+L488E variant
MGESVKAIKLKILDMFLDPECTKQDDNWRKDLSTMSRFCGEAGNMCLRDLYNYDSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNIEKPKKIRKAK

SEQ ID NO: 37 - protein, 497 aa, synthetic construct
  REGION 1..497: note = Cas-alpha 10 F54D+K85Q+T34E variant
MGESVKAIKLKILDMFLDPECTKQDDNWRKDLSEMSRFCGEAGNMCLRDLYNYDSMPKEDRISSKDLYNAMYHKTKLLHPGLPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 38 - protein, 497 aa, synthetic construct
  REGION 1..497: note = Cas-alpha 10 F54D+K85Q+S36A variant
MGESVKAIKLKILDMFLDPECTKQDDNWRKDLSTMARFCGEAGNMCLRDLYNYDSMPKEDRISSKDLYNAMYHKTKLLHPGLPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 39 - protein, 497 aa, synthetic construct
  REGION 1..497: note = Cas-alpha 10 F54D+K85Q+M45S variant
MGESVKAIKLKILDMFLDPECTKQDDNWRKDLSTMSRFCGEAGNSCLRDLYNYDSMPKEDRISSKDLYNAMYHKTKLLHPGLPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 40 - protein, 497 aa, synthetic construct
  REGION 1..497: note = Cas-alpha 10 F54D+K85Q+G470D variant
MGESVKAIKLKILDMFLDPECTKQDDNWRKDLSTMSRFCGEAGNMCLRDLYNYDSMPKEDRISSKDLYNAMYHKTKLLHPGLPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

SEQ ID NO: 41 - protein, 497 aa, synthetic construct
  REGION 1..497: note = Cas-alpha 10 F54D+K85Q+L488E variant
MGESVKAIKLKILDMFLDPECTKQDDNWRKDLSTMSRFCGEAGNMCLRDLYNYDSMPKEDRISSKDLYNAMYHKTKLLHPGLPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNIEKPKKIRKAK

SEQ ID NO: 42 - protein, 497 aa, synthetic construct
  REGION 1..497: note = Cas-alpha 10 F54D+K85Q+T34E+G470D variant
MGESVKAIKLKILDMFLDPECTKQDDNWRKDLSEMSRFCGEAGNMCLRDLYNYDSMPKEDRISSKDLYNAMYHKTKLLHPGLPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

SEQ ID NO: 43 - protein, 497 aa, synthetic construct
  REGION 1..497: note = Cas-alpha 10 F54D+K85Q+S36A+G470D variant
MGESVKAIKLKILDMFLDPECTKQDDNWRKDLSTMARFCGEAGNMCLRDLYNYDSMPKEDRISSKDLYNAMYHKTKLLHPGLPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

SEQ ID NO: 44 - protein, 497 aa, synthetic construct
  REGION 1..497: note = Cas-alpha 10 F54D+K85Q+M45S+G470D variant
MGESVKAIKLKILDMFLDPECTKQDDNWRKDLSTMSRFCGEAGNSCLRDLYNYDSMPKEDRISSKDLYNAMYHKTKLLHPGLPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

SEQ ID NO: 45 - protein, 497 aa, synthetic construct
  REGION 1..497: note = Cas-alpha 10 F54D+K85Q+T34E+L488E variant
MGESVKAIKLKILDMFLDPECTKQDDNWRKDLSEMSRFCGEAGNMCLRDLYNYDSMPKEDRISSKDLYNAMYHKTKLLHPGLPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNIEKPKKIRKAK

SEQ ID NO: 46 - protein, 497 aa, synthetic construct
  REGION 1..497: note = Cas-alpha 10 F54D+K85Q+S36A+L488E variant
MGESVKAIKLKILDMFLDPECTKQDDNWRKDLSTMARFCGEAGNMCLRDLYNYDSMPKEDRISSKDLYNAMYHKTKLLHPGLPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNIEKPKKIRKAK

SEQ ID NO: 47 - protein, 497 aa, synthetic construct
  REGION 1..497: note = Cas-alpha 10 F54D+K85Q+M45S+L488E variant
MGESVKAIKLKILDMFLDPECTKQDDNWRKDLSTMSRFCGEAGNSCLRDLYNYDSMPKEDRISSKDLYNAMYHKTKLLHPGLPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNIEKPKKIRKAK

SEQ ID NO: 48 - protein, 497 aa, Syntrophomonas palmitatica
MGESVKAIKLKILDMFLDPECTKQDDNWRKDLSTMSRFCAEAGNMCLRDLYNYFSMPKEDRISSKDLYNAMYHKTKLLHPELPGKVANQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYTQGQLQIKKDHQGKWYCIIPYTFPTHETVLDPDKVMGVDLGVAKAVYWAFNSSYKRGCIDGGEIEHFRKMIRARRVSIQNQIKHSGDARKGHGRKRALKPIETLSEKEKNFRDTINHRYANRIVEAAIKQGCGTIQIENLEGIADTTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 49 - protein, 497 aa, synthetic construct
MGESVKAIKLKILDMFLDPECTKQDDNWRKDLSEMSRFCGEAGNMCLRDLYNYDSMPKEDRISSKDLYNAMYHKTKLLHPELPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

SEQ ID NO: 50 - protein, 497 aa, synthetic construct
MGESVKAIKLKILDMFLDPECTKQDDNWRKDLSTMSRFCGEAGNSCLRDLYNYDSMPKEDRISSKDLYNAMYHKTKLLHPELPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

SEQ ID NO: 51 - protein, 497 aa, synthetic construct
MGESVKAIKLKILDMFLDPECTKQDDNWRKDLSEMSRFCGEAGNSCLRDLYNYDSMPKEDRISSKDLYNAMYHKTKLLHPGLPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

SEQ ID NO: 52 - protein, 497 aa, synthetic construct
MGESVKAIKLKILDMFLDPECTKQDDNWRKDLSEMARFCGEAGNMCLRDLYNYDSMPKEDRISSKDLYNAMYHKTKLLHPGLPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

SEQ ID NO: 53 - protein, 497 aa, synthetic construct
MGESVKAIKLKILDMFLDPECTKQDDNWRKDLSEMARFCGEAGNMCLRDLYNYDSMPKEDRISSKDLYNAMYHKTKLLHPELPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

SEQ ID NO: 54 - protein, 497 aa, synthetic construct
MGESVKAIKLKILDMFLDPECTKQDDNWRKDLSEMSRFCGEAGNSCLRDLYNYDSMPKEDRISSKDLYNAMYHKTKLLHPELPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

SEQ ID NO: 55 - protein, 497 aa, synthetic construct
MGESVKAIKLKILDMFLDPECTKQDDNWRKDLSEMARFCGEAGNSCLRDLYNYDSMPKEDRISSKDLYNAMYHKTKLLHPELPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

SEQ ID NO: 56 - protein, 497 aa, synthetic construct
MGESVKAIKLKILDMFLDPECTKQDDNWRKDLSEMARFCGEAGNSCLRDLYNYDSMPKEDRISSKDLYNAMYHKTKLLHPGLPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

SEQ ID NO: 57 - DNA, 24 bp, Saccharomyces cerevisiae
  source 1..24: mol_type = unassigned DNA
attctttagtgtaggaacatcaac

SEQ ID NO: 58 - DNA, 24 bp, Saccharomyces cerevisiae
  source 1..24: mol_type = unassigned DNA
gttcctacactaaagaatcttcaa

SEQ ID NO: 59 - protein, 497 aa, synthetic construct
MGESVKAIKLKILDMFLDPECTKQDDNWRKDLSTMSRFCGEAGNSCLRDLYNYDSMPKEDRISSKDLYNAMYHKTKLLHPELPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

SEQ ID NO: 60 - protein, 497 aa, synthetic construct
MGESVKAIKLKILDMFLDPECTKQDDNWRKDLSEMSRFCGEAGNSCLRDLYNYDSMPKEDRISSKDLYNAMYHKTKLLHPELPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

SEQ ID NO: 61 - RNA, 189 nt, synthetic construct
  source 1..189: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaac

SEQ ID NO: 62 - RNA, 209 nt, synthetic construct
  source 1..209: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 63 - RNA, 206 nt, synthetic construct
  source 1..206: mol_type = other RNA
tactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 64 - RNA, 206 nt, synthetic construct
  source 1..206: mol_type = other RNA
atctctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 65 - RNA, 206 nt, synthetic construct
  source 1..206: mol_type = other RNA
atttctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 66 - RNA, 206 nt, synthetic construct
  source 1..206: mol_type = other RNA
atttacgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 67 - RNA, 206 nt, synthetic construct
  source 1..206: mol_type = other RNA
atttactcttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 68 - RNA, 206 nt, synthetic construct
  source 1..206: mol_type = other RNA
atttactctgcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 69 - RNA, 206 nt, synthetic construct
  source 1..206: mol_type = other RNA
atttactctgttcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 70 - RNA, 206 nt, synthetic construct
  source 1..206: mol_type = other RNA
atttactctgtttccgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 71 - RNA, 206 nt, synthetic construct
  source 1..206: mol_type = other RNA
atttactctgtttcgcccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 72 - RNA, 206 nt, synthetic construct
  source 1..206: mol_type = other RNA
atttactctgtttcgcgcagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 73 - RNA, 206 nt, synthetic construct
  source 1..206: mol_type = other RNA
atttactctgtttcgcgcgcggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 74 - RNA, 206 nt, synthetic construct
  source 1..206: mol_type = other RNA
atttactctgtttcgcgcgccacagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 75 - RNA, 206 nt, synthetic construct
  source 1..206: mol_type = other RNA
atttactctgtttcgcgcgccagggttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 76 - RNA, 206 nt, synthetic construct
  source 1..206: mol_type = other RNA
atttactctgtttcgcgcgccagggctaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 77 - RNA, 206 nt, synthetic construct
  source 1..206: mol_type = other RNA
atttactctgtttcgcgcgccagggcagggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 78 - RNA, 206 nt, synthetic construct
  source 1..206: mol_type = other RNA
atttactctgtttcgcgcgccagggcagtttgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 79 - RNA, 206 nt, synthetic construct
  source 1..206: mol_type = other RNA
atttactctgtttcgcgcgccagggcagttagccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 80 - RNA, 206 nt, synthetic construct
  source 1..206: mol_type = other RNA
atttactctgtttcgcgcgccagggcagttaggtctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 81 - RNA, 206 nt, synthetic construct
  source 1..206: mol_type = other RNA
atttactctgtttcgcgcgccagggcagttaggtgcaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 82 - RNA, 206 nt, synthetic construct
  source 1..206: mol_type = other RNA
atttactctgtttcgcgcgccagggcagttaggtgcccaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 83 - RNA, 206 nt, synthetic construct
  source 1..206: mol_type = other RNA
atttactctgtttcgcgcgccagggcagttaggtgccctagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 84 - RNA, 206 nt, synthetic construct
  source 1..206: mol_type = other RNA
atttactctgtttcgcgcgccagggcagttaggtgccctaaagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 85 - RNA, 206 nt, synthetic construct
  source 1..206: mol_type = other RNA
atttactctgtttcgcgcgccagggcagttaggtgccctaaaaggaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 86 - RNA, 206 nt, synthetic construct
  source 1..206: mol_type = other RNA
atttactctgtttcgcgcgccagggcagttaggtgccctaaaagagagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 87 - RNA, 206 nt, synthetic construct
  source 1..206: mol_type = other RNA
atttactctgtttcgcgcgccagggcagttaggtgccctaaaagagcgtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 88 - RNA, 206 nt, synthetic construct
  source 1..206: mol_type = other RNA
atttactctgtttcgcgcgccagggcagttaggtgccctaaaagagcgaagccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 89 - RNA, 206 nt, synthetic construct
  source 1..206: mol_type = other RNA
atttactctgtttcgcgcgccagggcagttaggtgccctaaaagagcgaagtcgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 90 - RNA, 206 nt, synthetic construct
  source 1..206: mol_type = other RNA
atttactctgtttcgcgcgccagggcagttaggtgccctaaaagagcgaagtggaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 91 - RNA, 206 nt, synthetic construct
  source 1..206: mol_type = other RNA
atttactctgtttcgcgcgccagggcagttaggtgccctaaaagagcgaagtggccaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 92 - RNA, 206 nt, synthetic construct
  source 1..206: mol_type = other RNA
atttactctgtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 93 - RNA, 206 nt, synthetic construct
  source 1..206: mol_type = other RNA
atttactctgtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 94 - RNA, 206 nt, synthetic construct
  source 1..206: mol_type = other RNA
atttactctgtttcgcgcgccagggcagttaggtgccctaaaagagcgaagtggccgaaaggggctaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 95 - RNA, 206 nt, synthetic construct
  source 1..206: mol_type = other RNA
atttactctgtttcgcgcgccagggcagttaggtgccctaaaagagcgaagtggccgaaaggaactaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 96 - RNA, 206 nt, synthetic construct
  source 1..206: mol_type = other RNA
atttactctgtttcgcgcgccagggcagttaggtgccctaaaagagcgaagtggccgaaaggaaagaacgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 97 - RNA, 206 nt, synthetic construct
  source 1..206: mol_type = other RNA
atttactctgtttcgcgcgccagggcagttaggtgccctaaaagagcgaagtggccgaaaggaaaggccgcttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 98 - RNA, 206 nt, synthetic construct
  source 1..206: mol_type = other RNA
atttactctgtttcgcgcgccagggcagttaggtgccctaaaagagcgaagtggccgaaaggaaaggctacttctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 99 - RNA, 206 nt, synthetic construct
  source 1..206: mol_type = other RNA
atttactctgtttcgcgcgccagggcagttaggtgccctaaaagagcgaagtggccgaaaggaaaggctaactctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 100 - RNA, 206 nt, synthetic construct
  source 1..206: mol_type = other RNA
atttactctgtttcgcgcgccagggcagttaggtgccctaaaagagcgaagtggccgaaaggaaaggctaacgctctaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 101 - RNA, 206 nt, synthetic construct
  source 1..206: mol_type = other RNA
atttactctgtttcgcgcgccagggcagttaggtgccctaaaagagcgaagtggccgaaaggaaaggctaacgctttaacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 102 - RNA, 206 nt, synthetic construct
  source 1..206: mol_type = other RNA
atttactctgtttcgcgcgccagggcagttaggtgccctaaaagagcgaagtggccgaaaggaaaggctaacgcttctacgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 103 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctgctacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 104 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaatacggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 105 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgcggcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 106 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctgcgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 107 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacgaccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 108 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggccttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 109 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgttggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 110 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgacggcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 111 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgacctcgaaatgccatcaataccacgcggcccgaaagggttcgcgcgaaactgagtaatgaaagtcgcatcttgcgtaagcgcgtggattgaaactttagtgtaggaacatcaac

SEQ ID NO: 112 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttgaaatgccatcaataccacgcggcccgaaagggttcgcgcgaaactgagtaatgaaagtcgcatcttgcgtaagcgcgtggattgaaactttagtgtaggaacatcaac

SEQ ID NO: 113 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcatgccatcaataccacgcggcccgaaagggttcgcgcgaaactgagtaatgaaagtcgcatcttgcgtaagcgcgtggattgaaactttagtgtaggaacatcaac

SEQ ID NO: 114 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgagccatcaataccacgcggcccgaaagggttcgcgcgaaactgagtaatgaaagtcgcatcttgcgtaagcgcgtggattgaaactttagtgtaggaacatcaac

SEQ ID NO: 115 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaacatcaataccacgcggcccgaaagggttcgcgcgaaactgagtaatgaaagtcgcatcttgcgtaagcgcgtggattgaaactttagtgtaggaacatcaac

SEQ ID NO: 116 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgtcaataccacgcggcccgaaagggttcgcgcgaaactgagtaatgaaagtcgcatcttgcgtaagcgcgtggattgaaactttagtgtaggaacatcaac

SEQ ID NO: 117 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccaataccacgcggcccgaaagggttcgcgcgaaactgagtaatgaaagtcgcatcttgcgtaagcgcgtggattgaaactttagtgtaggaacatcaac

SEQ ID NO: 118 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccattaccacgcggcccgaaagggttcgcgcgaaactgagtaatgaaagtcgcatcttgcgtaagcgcgtggattgaaactttagtgtaggaacatcaac

SEQ ID NO: 119 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaccacgcggcccgaaagggttcgcgcgaaactgagtaatgaaagtcgcatcttgcgtaagcgcgtggattgaaactttagtgtaggaacatcaac

SEQ ID NO: 120 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaatacgcggcccgaaagggttcgcgcgaaactgagtaatgaaagtcgcatcttgcgtaagcgcgtggattgaaactttagtgtaggaacatcaac

SEQ ID NO: 121 - RNA, 206 nt, synthetic construct; identical to SEQ ID NO: 120
  source 1..206: mol_type = other RNA

SEQ ID NO: 122 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccaggcccgaaagggttcgcgcgaaactgagtaatgaaagtcgcatcttgcgtaagcgcgtggattgaaactttagtgtaggaacatcaac

SEQ ID NO: 123 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcccgaaagggttcgcgcgaaactgagtaatgaaagtcgcatcttgcgtaagcgcgtggattgaaactttagtgtaggaacatcaac

SEQ ID NO: 124 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcgcgaaagggttcgcgcgaaactgagtaatgaaagtcgcatcttgcgtaagcgcgtggattgaaactttagtgtaggaacatcaac

SEQ ID NO: 125 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcaaagggttcgcgcgaaactgagtaatgaaagtcgcatcttgcgtaagcgcgtggattgaaactttagtgtaggaacatcaac

SEQ ID NO: 126 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccagggttcgcgcgaaactgagtaatgaaagtcgcatcttgcgtaagcgcgtggattgaaactttagtgtaggaacatcaac

SEQ ID NO: 127 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaggttcgcgcgaaactgagtaatgaaagtcgcatcttgcgtaagcgcgtggattgaaactttagtgtaggaacatcaac

SEQ ID NO: 128 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaattcgcgcgaaactgagtaatgaaagtcgcatcttgcgtaagcgcgtggattgaaactttagtgtaggaacatcaac

SEQ ID NO: 129 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaaggcgcgcgaaactgagtaatgaaagtcgcatcttgcgtaagcgcgtggattgaaactttagtgtaggaacatcaac

SEQ ID NO: 130 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggtcgcgaaactgagtaatgaaagtcgcatcttgcgtaagcgcgtggattgaaactttagtgtaggaacatcaac

SEQ ID NO: 131 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttccgaaactgagtaatgaaagtcgcatcttgcgtaagcgcgtggattgaaactttagtgtaggaacatcaac

SEQ ID NO: 132 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcaaactgagtaatgaaagtcgcatcttgcgtaagcgcgtggattgaaactttagtgtaggaacatcaac

SEQ ID NO: 133 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcactgagtaatgaaagtcgcatcttgcgtaagcgcgtggattgaaactttagtgtaggaacatcaac

SEQ ID NO: 134 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgatgagtaatgaaagtcgcatcttgcgtaagcgcgtggattgaaactttagtgtaggaacatcaac

SEQ ID NO: 135 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaaagtaatgaaagtcgcatcttgcgtaagcgcgtggattgaaactttagtgtaggaacatcaac

SEQ ID NO: 136 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacttaatgaaagtcgcatcttgcgtaagcgcgtggattgaaactttagtgtaggaacatcaac

SEQ ID NO: 137 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgaatgaaagtcgcatcttgcgtaagcgcgtggattgaaactttagtgtaggaacatcaac

SEQ ID NO: 138 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtgaaagtcgcatcttgcgtaagcgcgtggattgaaactttagtgtaggaacatcaac

SEQ ID NO: 139 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaaaagtcgcatcttgcgtaagcgcgtggattgaaactttagtgtaggaacatcaac

SEQ ID NO: 140 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatggtcgcatcttgcgtaagcgcgtggattgaaactttagtgtaggaacatcaac

SEQ ID NO: 141 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaacgcatcttgcgtaagcgcgtggattgaaactttagtgtaggaacatcaac

SEQ ID NO: 142 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagcatcttgcgtaagcgcgtggattgaaactttagtgtaggaacatcaac

SEQ ID NO: 143 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtctcttgcgtaagcgcgtggattgaaactttagtgtaggaacatcaac

SEQ ID NO: 144 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcttgcgtaagcgcgtggattgaaactttagtgtaggaacatcaac

SEQ ID NO: 145 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatgcgtaagcgcgtggattgaaactttagtgtaggaacatcaac

SEQ ID NO: 146 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatctgtaagcgcgtggattgaaactttagtgtaggaacatcaac

SEQ ID NO: 147 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgaagcgcgtggattgaaactttagtgtaggaacatcaac

SEQ ID NO: 148 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcggcgcgtggattgaaactttagtgtaggaacatcaac

SEQ ID NO: 149 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtagcgtggattgaaactttagtgtaggaacatcaac

SEQ ID NO: 150 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaaggtggattgaaactttagtgtaggaacatcaac

SEQ ID NO: 151 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgggattgaaactttagtgtaggaacatcaac

SEQ ID NO: 152 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgattgaaactttagtgtaggaacatcaac

SEQ ID NO: 153 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtgtgaaactttagtgtaggaacatcaac

SEQ ID NO: 154 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggaaaactttagtgtaggaacatcaac

SEQ ID NO: 155 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattactttagtgtaggaacatcaac

SEQ ID NO: 156 - RNA, 206 nt, synthetic construct
  source 1..206: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgatttagtgtaggaacatcaac

SEQ ID NO: 157 - protein, 497 aa, Syntrophomonas palmitatica; identical to SEQ ID NO: 48

SEQ ID NO: 158 - DNA, 380 bp, Human betaherpesvirus 5
  source 1..380: mol_type = unassigned DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 159 - DNA, 204 bp, Human betaherpesvirus 5
  source 1..204: mol_type = unassigned DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 160 - DNA, 1491 bp, synthetic construct
  source 1..1491: mol_type = other DNA
atgggcgaaagcgtgaaagcaattaagctgaagatcctggatatgtttctggaccccgagtgcacgaagcaggatgataactggcgcaaagatttgtctactatgtccagattctgcggcgaggccgggaatatgtgcctgcgggacctgtataattactttagcatgcccaaggaagatcggatttccagcaaagacttatacaacgccatgtatcataaaactaaacttctccacccaggcctgccaggtaaggtcaagaatcaaatcgtgaaccacgctaaggatgtttggaaacgcaacgctaaactcatttatcggaatcaaatctcaatgcctacttataagatcacaaccgccccaatccggctgcagaataacatttataagttaatcaagaataagaacaagtacatcatagacgtgcagctgtactccaaggaatactccaaggatagtggtaagggcactcataggtatttcctggtcgccgttagagactcatctacccggatgatattcgataggattatgagtaaggaccatattgacagttctaagtcatacaagcagggacagctccagatcaagaaagaccaccaggggaagtggtattgcatcatcccctatactttccctacacatgagcacgtcctcgacccggataaagtcatgggagtcgaccttggcgtggccaaagctgtgtactgggcgttcaatagctcttacaaaagaggctgtatcgatggaggggagatcgagcatttccgcaaaatgatacgagcacgaagagtgagcatccaaaatcagatcaagcactcaggagacgcccgtaaaggacatgggcgcaaaagggccctaaaacccatagaaacattgagcgagaaggagagcaattttagggataccataaacttcagatatgcaaatcgaattgtagaagccgctattaagcagggctgtgggacgatccagatcgagaaccttgagggtatagctgacaggacaggcagtaaatttctcaagaactggccttactacgacctgcagacaaagattgtgaataaagccaaggaacacggcattaccgtggttgcaatcaacccccaatatacatcccaaaggtgctcgatgtgcgggtatattgaaaaaaccaaccgttctagccaggcagtgtttgagtgtaagcagtgcggttacggcagtcggactaactgtattaactgcaggcacgtccaagtatccggggatgtttgtgaggaatgtggcggcatcgtaaaaaaagaaaacgtgaacgcagactacaatgcggccaagaacatttccaccccgtacatcgaccagataatcatggagaagtgtctggaactagggattccttaccgcagcatcacctgtaaggaatgtggtcacatccaggcttcaggaaatacctgcgaggtgtgcggatctactaatattttgaagccaaagaaaattagaaaagccaaa

SEQ ID NO: 161 - DNA, 21 bp, synthetic construct
  source 1..21: mol_type = other DNA
ccaaagaagaagcggaaggtc

SEQ ID NO: 162 - DNA, 54 bp, synthetic construct
  source 1..54: mol_type = other DNA
gagggcagaggaagtctgctaacatgcggtgacgtcgaggagaatcctggccca

SEQ ID NO: 163 - RNA, 711 nt, synthetic construct
  source 1..711: mol_type = other RNA
agcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 164 - DNA, 123 bp, Human gammaherpesvirus 8
  source 1..123: mol_type = unassigned DNA
atcagcctcgactgtgccttctagttgccagccatctgttgtttgcccctcccccgtgccttccttgaccctggaaggtgccactcccactgtcctttcctaataaaatgaggaaattgcatc

SEQ ID NO: 165 - DNA, 250 bp, Homo sapiens
  source 1..250: mol_type = unassigned DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccg

SEQ ID NO: 166 - DNA, 176 bp, synthetic construct
  source 1..176: mol_type = other DNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaccgctgtcgcatcttgcgtaagcgcgtggattgaaac

SEQ ID NO: 167 - DNA, 159 bp, synthetic construct
  source 1..159: mol_type = other DNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacaaggtaagcgcgtggattgaaac

SEQ ID NO: 168 - DNA, 159 bp, synthetic construct
  source 1..159: mol_type = other DNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaataagcgcgtggattgaaac

SEQ ID NO: 169 - DNA, 156 bp, synthetic construct
  source 1..156: mol_type = other DNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacgaaataagcgcgtggattgaaac

SEQ ID NO: 170 - DNA, 174 bp, synthetic construct
  source 1..174: mol_type = other DNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaactgagtaatgaacgcatcttgcgtaagcgcgtggattgaaac

SEQ ID NO: 171 - DNA, 174 bp, synthetic construct
  source 1..174: mol_type = other DNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaactgagtaatgaaagtcgcatctgtaagcgcgtggattgaaac

SEQ ID NO: 172 - DNA, 171 bp, synthetic construct
  source 1..171: mol_type = other DNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaactgagtaatgaacgcatctgtaagcgcgtggattgaaac

SEQ ID NO: 173 - DNA, 20 bp, Homo sapiens
  source 1..20: mol_type = unassigned DNA
cctgtcttgtttgtgagagg

SEQ ID NO: 174 - DNA, 20 bp, Homo sapiens
  source 1..20: mol_type = unassigned DNA
ggttgtgttgccctccctgt

SEQ ID NO: 175 - skipped

SEQ ID NO: 176 - DNA, 209 bp, synthetic construct
  source 1..209: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaaccctgtcttgtttgtgagagg

SEQ ID NO: 177 - DNA, 196 bp, synthetic construct
  source 1..196: mol_type = other DNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaccgctgtcgcatcttgcgtaagcgcgtggattgaaaccctgtcttgtttgtgagagg

SEQ ID NO: 178 - DNA, 179 bp, synthetic construct
  source 1..179: mol_type = other DNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacaaggtaagcgcgtggattgaaaccctgtcttgtttgtgagagg

SEQ ID NO: 179 - DNA, 179 bp, synthetic construct
  source 1..179: mol_type = other DNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaataagcgcgtggattgaaaccctgtcttgtttgtgagagg

SEQ ID NO: 180 - DNA, 176 bp, synthetic construct
  source 1..176: mol_type = other DNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacgaaataagcgcgtggattgaaaccctgtcttgtttgtgagagg

SEQ ID NO: 181 - DNA, 194 bp, synthetic construct
  source 1..194: mol_type = other DNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaactgagtaatgaacgcatcttgcgtaagcgcgtggattgaaaccctgtcttgtttgtgagagg

SEQ ID NO: 182 - DNA, 194 bp, synthetic construct
  source 1..194: mol_type = other DNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaactgagtaatgaaagtcgcatctgtaagcgcgtggattgaaaccctgtcttgtttgtgagagg

SEQ ID NO: 183 - DNA, 191 bp, synthetic construct
  source 1..191: mol_type = other DNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaactgagtaatgaacgcatctgtaagcgcgtggattgaaaccctgtcttgtttgtgagagg

SEQ ID NO: 184 - DNA, 209 bp, synthetic construct
  source 1..209: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacggttgtgttgccctccctgt

SEQ ID NO: 185 - DNA, 196 bp, synthetic construct
  source 1..196: mol_type = other DNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaccgctgtcgcatcttgcgtaagcgcgtggattgaaacggttgtgttgccctccctgt

SEQ ID NO: 186 - DNA, 179 bp, synthetic construct
  source 1..179: mol_type = other DNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacaaggtaagcgcgtggattgaaacggttgtgttgccctccctgt

SEQ ID NO: 187 - DNA, 179 bp, synthetic construct
  source 1..179: mol_type = other DNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaataagcgcgtggattgaaacggttgtgttgccctccctgt

SEQ ID NO: 188 - DNA, 176 bp, synthetic construct
  source 1..176: mol_type = other DNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacgaaataagcgcgtggattgaaacggttgtgttgccctccctgt

SEQ ID NO: 189 - DNA, 194 bp, synthetic construct
  source 1..194: mol_type = other DNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaactgagtaatgaacgcatcttgcgtaagcgcgtggattgaaacggttgtgttgccctccctgt

SEQ ID NO: 190 - DNA, 194 bp, synthetic construct
  source 1..194: mol_type = other DNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaactgagtaatgaaagtcgcatctgtaagcgcgtggattgaaacggttgtgttgccctccctgt

SEQ ID NO: 191 - DNA, 191 bp, synthetic construct
  source 1..191: mol_type = other DNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaactgagtaatgaacgcatctgtaagcgcgtggattgaaacggttgtgttgccctccctgt

SEQ ID NO: 192 - RNA, 209 nt, synthetic construct
  source 1..209: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaaccctgtcttgtttgtgagagg

SEQ ID NO: 193 - RNA, 196 nt, synthetic construct
  source 1..196: mol_type = other RNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaccgctgtcgcatcttgcgtaagcgcgtggattgaaaccctgtcttgtttgtgagagg

SEQ ID NO: 194 - RNA, 179 nt, synthetic construct
  source 1..179: mol_type = other RNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacaaggtaagcgcgtggattgaaaccctgtcttgtttgtgagagg

SEQ ID NO: 195 - RNA, 179 nt, synthetic construct
  source 1..179: mol_type = other RNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaataagcgcgtggattgaaaccctgtcttgtttgtgagagg

SEQ ID NO: 196 - RNA, 176 nt, synthetic construct
  source 1..176: mol_type = other RNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacgaaataagcgcgtggattgaaaccctgtcttgtttgtgagagg

SEQ ID NO: 197 - RNA, 194 nt, synthetic construct
  source 1..194: mol_type = other RNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaactgagtaatgaacgcatcttgcgtaagcgcgtggattgaaaccctgtcttgtttgtgagagg

SEQ ID NO: 198 - RNA, 194 nt, synthetic construct
  source 1..194: mol_type = other RNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaactgagtaatgaaagtcgcatctgtaagcgcgtggattgaaaccctgtcttgtttgtgagagg

SEQ ID NO: 199 - RNA, 191 nt, synthetic construct
  source 1..191: mol_type = other RNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaactgagtaatgaacgcatctgtaagcgcgtggattgaaaccctgtcttgtttgtgagagg

SEQ ID NO: 200 - RNA, 209 nt, synthetic construct
  source 1..209: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacggttgtgttgccctccctgt

SEQ ID NO: 201 - RNA, 196 nt, synthetic construct
  source 1..196: mol_type = other RNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaccgctgtcgcatcttgcgtaagcgcgtggattgaaacggttgtgttgccctccctgt

SEQ ID NO: 202 - RNA, 179 nt, synthetic construct
  source 1..179: mol_type = other RNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacaaggtaagcgcgtggattgaaacggttgtgttgccctccctgt

SEQ ID NO: 203 - RNA, 179 nt, synthetic construct
  source 1..179: mol_type = other RNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaataagcgcgtggattgaaacggttgtgttgccctccctgt

SEQ ID NO: 204 - RNA, 176 nt, synthetic construct
  source 1..176: mol_type = other RNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacgaaataagcgcgtggattgaaacggttgtgttgccctccctgt

SEQ ID NO: 205 - RNA, 194 nt, synthetic construct
  source 1..194: mol_type = other RNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaactgagtaatgaacgcatcttgcgtaagcgcgtggattgaaacggttgtgttgccctccctgt

SEQ ID NO: 206 - RNA, 194 nt, synthetic construct
  source 1..194: mol_type = other RNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaactgagtaatgaaagtcgcatctgtaagcgcgtggattgaaacggttgtgttgccctccctgt

SEQ ID NO: 207 - RNA, 191 nt, synthetic construct
  source 1..191: mol_type = other RNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaactgagtaatgaacgcatctgtaagcgcgtggattgaaacggttgtgttgccctccctgt

SEQ ID NO: 208 - RNA, 21 nt, Emesvirus zinderi
  source 1..21: mol_type = unassigned RNA
aacatgaggatcacccatgtc

SEQ ID NO: 209 - RNA, 17 nt, Pseudomonas aeruginosa
  source 1..17: mol_type = unassigned RNA
actgccgtataggcagc

SEQ ID NO: 210 - DNA, 223 bp, synthetic construct
  source 1..223: mol_type = other DNA
ggccaacatgaggatcacccatgtctgcagggccatttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaac

SEQ ID NO: 211 - DNA, 217 bp, synthetic construct
  source 1..217: mol_type = other DNA
atttactctgtttcgcgcgccagggcaggccaacatgaggatcacccatgtctgcagggcctgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaac

SEQ ID NO: 212 - DNA, 214 bp, synthetic construct
  source 1..214: mol_type = other DNA
atttactctgtttcgcgcgccagggcagttaggtgccctaaaagagcgaagtggccggccaacatgaggatcacccatgtctgcagggccggctaacgcttctctaacgctacggcgaccttggcgaaatgccatcaataccacgcggcccgaaagggttcgcgcgaaactgagtaatgaaagtcgcatcttgcgtaagcgcgtggattgaaac

SEQ ID NO: 213 - DNA, 206 bp, synthetic construct
  source 1..206: mol_type = other DNA
atttactctgtttcgcgcgccagggcagttaggtgccctaaaagagcgaagtggccaacatgaggatcacccatgtctgcagggctaacgcttctctaacgctacggcgaccttggcgaaatgccatcaataccacgcggcccgaaagggttcgcgcgaaactgagtaatgaaagtcgcatcttgcgtaagcgcgtggattgaaac

SEQ ID NO: 214 - DNA, 220 bp, synthetic construct
  source 1..220: mol_type = other DNA
atttactctgtttcgcgcgccagggcagttaggtgccctaaaagagcgaagtggccgaggccaacatgaggatcacccatgtctgcagggccgaaaggctaacgcttctctaacgctacggcgaccttggcgaaatgccatcaataccacgcggcccgaaagggttcgcgcgaaactgagtaatgaaagtcgcatcttgcgtaagcgcgtggattgaaac

SEQ ID NO: 215 - DNA, 220 bp, synthetic construct
  source 1..220: mol_type = other DNA
atttactctgtttcgcgcgccagggcagttaggtgccctaaaagagcgaagtggccgaaaggccaacatgaggatcacccatgtctgcagggccaaggctaacgcttctctaacgctacggcgaccttggcgaaatgccatcaataccacgcggcccgaaagggttcgcgcgaaactgagtaatgaaagtcgcatcttgcgtaagcgcgtggattgaaac

SEQ ID NO: 216 - DNA, 212 bp, synthetic construct
  source 1..212: mol_type = other DNA
atttactctgtttcgcgcgccagggcagttaggtgccctaaaagagcgaagtggccgaaaggaaaggctaacgcttctctaacgcgaacatgaggatcacccatgtctacacgcgaccttggcgaaatgccatcaataccacgcggcccgaaagggttcgcgcgaaactgagtaatgaaagtcgcatcttgcgtaagcgcgtggattgaaac

SEQ ID NO: 217 - DNA, 212 bp, synthetic construct
  source 1..212: mol_type = other DNA
atttactctgtttcgcgcgccagggcagttaggtgccctaaaagagcgaagtggccgaaaggaaaggctaacgcttctctaacgctacggcgaccttggcgaacatgaggatcacccatgtcatcagcgccatcaataccacgcggcccgaaagggttcgcgcgaaactgagtaatgaaagtcgcatcttgcgtaagcgcgtggattgaaac

SEQ ID NO: 218 - DNA, 211 bp, synthetic construct
  source 1..211: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccaacatgaggatcacccatgtcatcaagggttcgcgcgaaactgagtaatgaaagtcgcatcttgcgtaagcgcgtggattgaaac

SEQ ID NO: 219 - DNA, 206 bp, synthetic construct
  source 1..206: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaacatgaggatcacccatgtcaaagtcgcatcttgcgtaagcgcgtggattgaaac

SEQ ID NO: 220 - DNA, 212 bp, synthetic construct
  source 1..212: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaaacatgaggatcacccatgtcatgaacgcatcttgcgtaagcgcgtggattgaaac

SEQ ID NO: 221 - DNA, 223 bp, synthetic construct
  source 1..223: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgggccaacatgaggatcacccatgtctgcagggcctaagcgcgtggattgaaac

SEQ ID NO: 222 - DNA, 218 bp, synthetic construct
  source 1..218: mol_type = other DNA
ggccacactgccgtataggcagctaggccatttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaac

SEQ ID NO: 223 - DNA, 212 bp, synthetic construct
  source 1..212: mol_type = other DNA
atttactctgtttcgcgcgccagggcaggccacactgccgtataggcagctaggcctgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaac

SEQ ID NO: 224 - DNA, 209 bp, synthetic construct
  source 1..209: mol_type = other DNA
atttactctgtttcgcgcgccagggcagttaggtgccctaaaagagcgaagtggccggccacactgccgtataggcagctaggccggctaacgcttctctaacgctacggcgaccttggcgaaatgccatcaataccacgcggcccgaaagggttcgcgcgaaactgagtaatgaaagtcgcatcttgcgtaagcgcgtggattgaaac

SEQ ID NO: 225 - DNA, 201 bp, synthetic construct
  source 1..201: mol_type = other DNA
atttactctgtttcgcgcgccagggcagttaggtgccctaaaagagcgaagtggccacactgccgtataggcagctaggctaacgcttctctaacgctacggcgaccttggcgaaatgccatcaataccacgcggcccgaaagggttcgcgcgaaactgagtaatgaaagtcgcatcttgcgtaagcgcgtggattgaaac

SEQ ID NO: 226 - DNA, 215 bp, synthetic construct
  source 1..215: mol_type = other DNA
atttactctgtttcgcgcgccagggcagttaggtgccctaaaagagcgaagtggccgaggccacactgccgtataggcagctaggccgaaaggctaacgcttctctaacgctacggcgaccttggcgaaatgccatcaataccacgcggcccgaaagggttcgcgcgaaactgagtaatgaaagtcgcatcttgcgtaagcgcgtggattgaaac

SEQ ID NO: 227 - DNA, 215 bp, synthetic construct
  source 1..215: mol_type = other DNA
atttactctgtttcgcgcgccagggcagttaggtgccctaaaagagcgaagtggccgaaaggccacactgccgtataggcagctaggccaaggctaacgcttctctaacgctacggcgaccttggcgaaatgccatcaataccacgcggcccgaaagggttcgcgcgaaactgagtaatgaaagtcgcatcttgcgtaagcgcgtggattgaaac

SEQ ID NO: 228 - DNA, 208 bp, synthetic construct
  source 1..208: mol_type = other DNA
atttactctgtttcgcgcgccagggcagttaggtgccctaaaagagcgaagtggccgaaaggaaaggctaacgcttctctaacgcgacactgccgtataggcagctacgcgaccttggcgaaatgccatcaataccacgcggcccgaaagggttcgcgcgaaactgagtaatgaaagtcgcatcttgcgtaagcgcgtggattgaaac

SEQ ID NO: 229 - DNA, 207 bp, synthetic construct
  source 1..207: mol_type = other DNA
atttactctgtttcgcgcgccagggcagttaggtgccctaaaagagcgaagtggccgaaaggaaaggctaacgcttctctaacgctacggcgaccttggcgacactgccgtataggcagctacgccatcaataccacgcggcccgaaagggttcgcgcgaaactgagtaatgaaagtcgcatcttgcgtaagcgcgtggattgaaac

SEQ ID NO: 230 - DNA, 206 bp, synthetic construct
  source 1..206: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccacactgccgtataggcagctagggttcgcgcgaaactgagtaatgaaagtcgcatcttgcgtaagcgcgtggattgaaac

SEQ ID NO: 231 - DNA, 206 bp, synthetic construct
  source 1..206: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtacactgccgtataggcagctaaaagtcgcatcttgcgtaagcgcgtggattgaaac

SEQ ID NO: 232 - DNA, 207 bp, synthetic construct
  source 1..207: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaacactgccgtataggcaggaacgcatcttgcgtaagcgcgtggattgaaac

SEQ ID NO: 233 - DNA, 218 bp, synthetic construct
  source 1..218: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgggccacactgccgtataggcagctaggcctaagcgcgtggattgaaac

SEQ ID NO: 234 - DNA, 20 bp, Homo sapiens
  source 1..20: mol_type = unassigned DNA
accgagcaggagtgagggaa

SEQ ID NO: 235 - DNA, 209 bp, synthetic construct
  source 1..209: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 236 - DNA, 243 bp, synthetic construct
  source 1..243: mol_type = other DNA
ggccaacatgaggatcacccatgtctgcagggccatttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 237 - DNA, 237 bp, synthetic construct
  source 1..237: mol_type = other DNA
atttactctgtttcgcgcgccagggcaggccaacatgaggatcacccatgtctgcagggcctgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 238 - DNA, 234 bp, synthetic construct
  source 1..234: mol_type = other DNA
atttactctgtttcgcgcgccagggcagttaggtgccctaaaagagcgaagtggccggccaacatgaggatcacccatgtctgcagggccggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 239 - DNA, 226 bp, synthetic construct
  source 1..226: mol_type = other DNA
atttactctgtttcgcgcgccagggcagttaggtgccctaaaagagcgaagtggccaacatgaggatcacccatgtctgcagggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 240 - DNA, 240 bp, synthetic construct
  source 1..240: mol_type = other DNA
atttactctgtttcgcgcgccagggcagttaggtgccctaaaagagcgaagtggccgaggccaacatgaggatcacccatgtctgcagggccgaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 241 - DNA, 240 bp, synthetic construct
  source 1..240: mol_type = other DNA
atttactctgtttcgcgcgccagggcagttaggtgccctaaaagagcgaagtggccgaaaggccaacatgaggatcacccatgtctgcagggccaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 242 - DNA, 232 bp, synthetic construct
  source 1..232: mol_type = other DNA
atttactctgtttcgcgcgccagggcagttaggtgccctaaaagagcgaagtggccgaaaggaaaggctaacgcttctctaacgcgaacatgaggatcacccatgtctacacgcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 243 - DNA, 232 bp, synthetic construct
  source 1..232: mol_type = other DNA
atttactctgtttcgcgcgccagggcagttaggtgccctaaaagagcgaagtggccgaaaggaaaggctaacgcttctctaacgctacggcgaccttggcgaacatgaggatcacccatgtcatcagcgccatcaataccacgcggcccgaaagggttcgcgcgaaactgagtaatgaaagtcgcatcttgcgtaagcgcgtggattgaaacaccgagcaggagtgagggaa

SEQ ID NO: 244 - DNA, 231 bp, synthetic construct
  source 1..231: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccaacatgaggatcacccatgtcatcaagggttcgcgcgaaactgagtaatgaaagtcgcatcttgcgtaagcgcgtggattgaaacaccgagcaggagtgagggaa

SEQ ID NO: 245 - DNA, 226 bp, synthetic construct
  source 1..226: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaacatgaggatcacccatgtcaaagtcgcatcttgcgtaagcgcgtggattgaaacaccgagcaggagtgagggaa

SEQ ID NO: 246 - DNA, 232 bp, synthetic construct
  source 1..232: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaaacatgaggatcacccatgtcatgaacgcatcttgcgtaagcgcgtggattgaaacaccgagcaggagtgagggaa

SEQ ID NO: 247 - DNA, 243 bp, synthetic construct
  source 1..243: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgggccaacatgaggatcacccatgtctgcagggcctaagcgcgtggattgaaacaccgagcaggagtgagggaa

SEQ ID NO: 248 - DNA, 238 bp, synthetic construct
  source 1..238: mol_type = other DNA
ggccacactgccgtataggcagctaggccatttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 249 - DNA, 232 bp, synthetic construct
  source 1..232: mol_type = other DNA
atttactctgtttcgcgcgccagggcaggccacactgccgtataggcagctaggcctgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 250 - DNA, 229 bp, synthetic construct
  source 1..229: mol_type = other DNA
atttactctgtttcgcgcgccagggcagttaggtgccctaaaagagcgaagtggccggccacactgccgtataggcagctaggccggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 251 - DNA, 221 bp, synthetic construct
  source 1..221: mol_type = other DNA
atttactctgtttcgcgcgccagggcagttaggtgccctaaaagagcgaagtggccacactgccgtataggcagctaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 252 - DNA, 235 bp, synthetic construct
  source 1..235: mol_type = other DNA
atttactctgtttcgcgcgccagggcagttaggtgccctaaaagagcgaagtggccgaggccacactgccgtataggcagctaggccgaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 253 - DNA, 235 bp, synthetic construct
  source 1..235: mol_type = other DNA
atttactctgtttcgcgcgccagggcagttaggtgccctaaaagagcgaagtggccgaaaggccacactgccgtataggcagctaggccaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 254 - DNA, 228 bp, synthetic construct
  source 1..228: mol_type = other DNA
atttactctgtttcgcgcgccagggcagttaggtgccctaaaagagcgaagtggccgaaaggaaaggctaacgcttctctaacgcgacactgccgtataggcagctacgcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 255 - DNA, 227 bp, synthetic construct
  source 1..227: mol_type = other DNA
atttactctgtttcgcgcgccagggcagttaggtgccctaaaagagcgaagtggccgaaaggaaaggctaacgcttctctaacgctacggcgaccttggcgacactgccgtataggcagctacgccatcaataccacgcggcccgaaagggttcgcgcgaaactgagtaatgaaagtcgcatcttgcgtaagcgcgtggattgaaacaccgagcaggagtgagggaa

SEQ ID NO: 256 - DNA, 226 bp, synthetic construct
  source 1..226: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccacactgccgtataggcagctagggttcgcgcgaaactgagtaatgaaagtcgcatcttgcgtaagcgcgtggattgaaacaccgagcaggagtgagggaa

SEQ ID NO: 257 - DNA, 226 bp, synthetic construct
  source 1..226: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtacactgccgtataggcagctaaaagtcgcatcttgcgtaagcgcgtggattgaaacaccgagcaggagtgagggaa

SEQ ID NO: 258 - DNA, 227 bp, synthetic construct
  source 1..227: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaacactgccgtataggcaggaacgcatcttgcgtaagcgcgtggattgaaacaccgagcaggagtgagggaa

SEQ ID NO: 259 - DNA, 238 bp, synthetic construct
  source 1..238: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgggccacactgccgtataggcagctaggcctaagcgcgtggattgaaacaccgagcaggagtgagggaa

SEQ ID NO: 260 - RNA, 209 nt, synthetic construct
  source 1..209: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 261 - RNA, 243 nt, synthetic construct
  source 1..243: mol_type = other RNA
ggccaacatgaggatcacccatgtctgcagggccatttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 262 - RNA, 237 nt, synthetic construct
  source 1..237: mol_type = other RNA
atttactctgtttcgcgcgccagggcaggccaacatgaggatcacccatgtctgcagggcctgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 263 - RNA, 234 nt, synthetic construct
  source 1..234: mol_type = other RNA
atttactctgtttcgcgcgccagggcagttaggtgccctaaaagagcgaagtggccggccaacatgaggatcacccatgtctgcagggccggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 264 - RNA, 226 nt, synthetic construct
  source 1..226: mol_type = other RNA
atttactctgtttcgcgcgccagggcagttaggtgccctaaaagagcgaagtggccaacatgaggatcacccatgtctgcagggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 265 - RNA, 240 nt, synthetic construct
  source 1..240: mol_type = other RNA
atttactctgtttcgcgcgccagggcagttaggtgccctaaaagagcgaagtggccgaggccaacatgaggatcacccatgtctgcagggccgaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 266 - RNA, 240 nt, synthetic construct
  source 1..240: mol_type = other RNA
atttactctgtttcgcgcgccagggcagttaggtgccctaaaagagcgaagtggccgaaaggccaacatgaggatcacccatgtctgcagggccaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 267 - RNA, 232 nt, synthetic construct
  source 1..232: mol_type = other RNA
atttactctgtttcgcgcgccagggcagttaggtgccctaaaagagcgaagtggccgaaaggaaaggctaacgcttctctaacgcgaacatgaggatcacccatgtctacacgcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 268 - RNA, 232 nt, synthetic construct
  source 1..232: mol_type = other RNA
atttactctgtttcgcgcgccagggcagttaggtgccctaaaagagcgaagtggccgaaaggaaaggctaacgcttctctaacgctacggcgaccttggcgaacatgaggatcacccatgtcatcagcgccatcaataccacgcggcccgaaagggttcgcgcgaaactgagtaatgaaagtcgcatcttgcgtaagcgcgtggattgaaacaccgagcaggagtgagggaa

SEQ ID NO: 269 - RNA, 231 nt, synthetic construct
  source 1..231: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccaacatgaggatcacccatgtcatcaagggttcgcgcgaaactgagtaatgaaagtcgcatcttgcgtaagcgcgtggattgaaacaccgagcaggagtgagggaa

SEQ ID NO: 270 - RNA, 226 nt, synthetic construct
  source 1..226: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaacatgaggatcacccatgtcaaagtcgcatcttgcgtaagcgcgtggattgaaacaccgagcaggagtgagggaa

SEQ ID NO: 271 - RNA, 232 nt, synthetic construct
  source 1..232: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaaacatgaggatcacccatgtcatgaacgcatcttgcgtaagcgcgtggattgaaacaccgagcaggagtgagggaa

SEQ ID NO: 272 - RNA, 243 nt, synthetic construct
  source 1..243: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgggccaacatgaggatcacccatgtctgcagggcctaagcgcgtggattgaaacaccgagcaggagtgagggaa

SEQ ID NO: 273 - RNA, 238 nt, synthetic construct
  source 1..238: mol_type = other RNA
ggccacactgccgtataggcagctaggccatttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 274 - RNA, 232 nt, synthetic construct
  source 1..232: mol_type = other RNA
atttactctgtttcgcgcgccagggcaggccacactgccgtataggcagctaggcctgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 275 - RNA, 229 nt, synthetic construct
  source 1..229: mol_type = other RNA
atttactctgtttcgcgcgccagggcagttaggtgccctaaaagagcgaagtggccggccacactgccgtataggcagctaggccggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 276 - RNA, 221 nt, synthetic construct
  source 1..221: mol_type = other RNA
atttactctgtttcgcgcgccagggcagttaggtgccctaaaagagcgaagtggccacactgccgtataggcagctaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 277 - RNA, 235 nt, synthetic construct
  source 1..235: mol_type = other RNA
atttactctgtttcgcgcgccagggcagttaggtgccctaaaagagcgaagtggccgaggccacactgccgtataggcagctaggccgaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 278 - RNA, 235 nt, synthetic construct
  source 1..235: mol_type = other RNA
atttactctgtttcgcgcgccagggcagttaggtgccctaaaagagcgaagtggccgaaaggccacactgccgtataggcagctaggccaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 279 - RNA, 228 nt, synthetic construct
  source 1..228: mol_type = other RNA
atttactctgtttcgcgcgccagggcagttaggtgccctaaaagagcgaagtggccgaaaggaaaggctaacgcttctctaacgcgacactgccgtataggcagctacgcgaccttggcgaaatgccatcaataccacgcggcccgaaagggttcgcgcgaaactgagtaatgaaagtcgcatcttgcgtaagcgcgtggattgaaacaccgagcaggagtgagggaa

SEQ ID NO: 280 - RNA, 227 nt, synthetic construct
  source 1..227: mol_type = other RNA
atttactctgtttcgcgcgccagggcagttaggtgccctaaaagagcgaagtggccgaaaggaaaggctaacgcttctctaacgctacggcgaccttggcgacactgccgtataggcagctacgccatcaataccacgcggcccgaaagggttcgcgcgaaactgagtaatgaaagtcgcatcttgcgtaagcgcgtggattgaaacaccgagcaggagtgagggaa

SEQ ID NO: 281 - RNA, 226 nt, synthetic construct
  source 1..226: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccacactgccgtataggcagctagggttcgcgcgaaactgagtaatgaaagtcgcatcttgcgtaagcgcgtggattgaaacaccgagcaggagtgagggaa

SEQ ID NO: 282 - RNA, 226 nt, synthetic construct
  source 1..226: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtacactgccgtataggcagctaaaagtcgcatcttgcgtaagcgcgtggattgaaacaccgagcaggagtgagggaa

SEQ ID NO: 283 - RNA, 227 nt, synthetic construct
  source 1..227: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaacactgccgtataggcaggaacgcatcttgcgtaagcgcgtggattgaaacaccgagcaggagtgagggaa

SEQ ID NO: 284 - RNA, 238 nt, synthetic construct
  source 1..238: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgggccacactgccgtataggcagctaggcctaagcgcgtggattgaaacaccgagcaggagtgagggaa

SEQ ID NO: 285 - DNA, 896 bp, Zea mays
  source 1..896: mol_type = other DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctt

SEQ ID NO: 286 - DNA, 82 bp, Zea mays
  source 1..82: mol_type = other DNA
tccccaacctcgtgttgttcggagcgcacacacacacaaccagatctcccccaaatccacccgtcggcacctccgcttcaag

SEQ ID NO: 287 - DNA, 1013 bp, Zea mays
  source 1..1013: mol_type = other DNA
gtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 288 - DNA, 189 bp, Solanum tuberosum
  source 1..189: mol_type = other DNA
gtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcag

SEQ ID NO: 289 - DNA, 1680 bp, synthetic construct
  source 1..1680: mol_type = other DNA
atgggcgagtccgtgaaggccatcaagctcaagatcctggacatgttcctcgacccggagtgcaccaagcaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggacgacaactggaggaaggacctctccaccatgagccgcttctgcggcgaggctggcaacatgtgcctcagggacctgtacaactacttctccatgccgaaggaggaccgcatctccagcaaggacctctacaacgcgatgtaccacaagacgaagctcctgcaccccggcctgcccggcaaggtgaagaaccagatcgtcaaccacgccaaggacgtgtggaagaggaacgcgaagctgatctaccgcaaccagatctccatgccgacgtacaagatcaccacggcccccatcaggctccagaacaacatctacaagctgatcaagaacaagaacaagtacatcatcgacgtccagctctacagcaaggagtactccaaggacagcggcaagggcacccaccgctacttcctggtggccgtccgcgactccagcacgaggatgatcttcgaccgcatcatgtccaaggaccacatcgactccagcaagagctacaagcagggccagctccagatcaagaaggaccaccagggcaagtggtactgcatcatcccgtacaccttcccgacccacgagcacgtgctcgacccggacaaggtcatgggcgtggacctcggcgtggctaaggccgtctactgggccttcaactccagctacaagcggggctgcatcgacggcggcgagatcgagcacttccgcaagatgatcagggcgcgcagggtctccatccagaaccagatcaagcactcgggcgacgctaggaagggccacggccgcaagagggcgctcaagcccatcgagaccctgtccgagaaggagagcaacttccgcgacacgatcaacttccgctacgccaacaggatcgtggaggcggcgatcaagcagggctgcggcaccatccagatcgagaacctggagggcatcgccgacaggacgggcagcaagttcctcaagaactggccgtactacgacctgcagaccaagatcgtcaacaaggccaaggagcacggcatcaccgtggtcgcgatcaacccccagtacacgtcccagaggtgcagcatgtgcggctacatcgagaagaccaaccgctccagccaggccgtgttcgagtgcaagcagtgcggctacggctccaggacgaactgcatcaactgccgccacgtgcaggtcagcggcgacgtctgcgaggagtgcggtggcatcgtgaagaaggagaacgtcaacgcggactacaacgccgcgaagaacatctccaccccgtacatcgaccagatcatcatggagaagtgcctggagctgggcatcccctacaggagcatcacgtgcaaggagtgcggccacatccaggcctccggcaacacctgcgaggtgtgcggcagcacgaacatcctcaagcccaagaagatccgcaaggcgaag

SEQ ID NO: 290 - DNA, 21 bp, synthetic construct
  source 1..21: mol_type = other DNA
ccgaagaagaagcgcaaggtg

SEQ ID NO: 291 - DNA, 910 bp, Zea mays
  source 1..910: mol_type = other DNA
gtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgac

SEQ ID NO: 292 - DNA, 1001 bp, synthetic construct
  source 1..1001: mol_type = other DNA
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattg

SEQ ID NO: 293 - DNA, 20 bp, Zea mays
  source 1..20: mol_type = other DNA
aagttcacggcgttccaggc

SEQ ID NO: 294 - DNA, 20 bp, Zea mays
  source 1..20: mol_type = other DNA
cacagggagggagcaaacaa

SEQ ID NO: 295 - DNA, 20 bp, Zea mays
  source 1..20: mol_type = other DNA
ggttctgggatagcttggga

SEQ ID NO: 296 - skipped

SEQ ID NO: 297 - DNA, 209 bp, synthetic construct
  source 1..209: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaagttcacggcgttccaggc

SEQ ID NO: 298 - DNA, 209 bp, synthetic construct
  source 1..209: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaaccacagggagggagcaaacaa

SEQ ID NO: 299 - DNA, 209 bp, synthetic construct
  source 1..209: mol_type = other DNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacggttctgggatagcttggga

SEQ ID NO: 300 - DNA, 179 bp, synthetic construct
  source 1..179: mol_type = other DNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaataagcgcgtggattgaaacaagttcacggcgttccaggc

SEQ ID NO: 301 - DNA, 179 bp, synthetic construct
  source 1..179: mol_type = other DNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaataagcgcgtggattgaaaccacagggagggagcaaacaa

SEQ ID NO: 302 - DNA, 179 bp, synthetic construct
  source 1..179: mol_type = other DNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaataagcgcgtggattgaaacggttctgggatagcttggga

SEQ ID NO: 303 - DNA, 24 bp, Zea mays
  source 1..24: mol_type = other DNA
gttcaagttcacggcgttccaggc

SEQ ID NO: 304 - DNA, 24 bp, Zea mays
  source 1..24: mol_type = other DNA
gttccacagggagggagcaaacaa

SEQ ID NO: 305 - DNA, 24 bp, Zea mays
  source 1..24: mol_type = other DNA
tttcggttctgggatagcttggga

SEQ ID NO: 306 - DNA, 24 bp, Homo sapiens
  source 1..24: mol_type = other DNA
gttccctgtcttgtttgtgagagg

SEQ ID NO: 307 - DNA, 24 bp, Homo sapiens
  source 1..24: mol_type = other DNA
cttcggttgtgttgccctccctgt

SEQ ID NO: 308 - DNA, 24 bp, Homo sapiens
  source 1..24: mol_type = other DNA
attcaccgagcaggagtgagggaa

SEQ ID NO: 309 - protein, 497 aa, synthetic construct
MGESVKAIKLKILDMFLDPECTKQDDNWRKDLSTMSRFCAEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 310 - protein, 497 aa, synthetic construct
MGESVKAIKLKILDMFLDPECTKQDDNWRKDLSTMSRFCGEAGNMCLRDLYNYFSMPKEDRISSKDLYNAMYHKTKLLHPGLPGKVANQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 311 - protein, 497 aa, synthetic construct
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYTQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 312 - protein, 497 aa, synthetic construct
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHETVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 313 - protein, 497 aa, synthetic construct
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKEKNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 314 - protein, 497 aa, synthetic construct
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINHRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 315 - protein, 497 aa, synthetic construct
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADTTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 316 - protein, 497 aa, synthetic construct
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 317 - protein, 497 aa, synthetic construct
MGESVKAIKLKILDMFLDPECTKQDDNWRKDLSTMSRFCGEAGNMCLRDLYNYFSMPKEDRISSKDLYNAMYHKTKLLHPELPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKEKNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 318 - protein, 497 aa, synthetic construct
MGESVKAIKLKILDMFLDPECTKQDDNWRKDLSTMSRFCGEAGNMCLRDLYNYFSMPKEDRISSKDLYNAMYHKTKLLHPELPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKEKNFRDTINHRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 319 - protein, 497 aa, synthetic construct
MGESVKAIKLKILDMFLDPECTKQDDNWRKDLSTMSRFCGEAGNMCLRDLYNYFSMPKEDRISSKDLYNAMYHKTKLLHPELPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKEKNFRDTINHRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 320 - protein, 497 aa, synthetic construct
MGESVKAIKLKILDMFLDPECTKQDDNWRKDLSTMSRFCGEAGNMCLRDLYNYFSMPKEDRISSKDLYNAMYHKTKLLHPELPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKEKNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 321 - protein, 497 aa, synthetic construct
MGESVKAIKLKILDMFLDPECTKQDDNWRKDLSTMSRFCGEAGNMCLRDLYNYFSMPKEDRISSKDLYNAMYHKTKLLHPELPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 322 - protein, 497 aa, synthetic construct
MGESVKAIKLKILDMFLDPECTKQDDNWRKDLSTMSRFCGEAGNMCLRDLYNYFSMPKEDRISSKDLYNAMYHKTKLLHPELPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINHRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 323 - protein, 497 aa, synthetic construct
MGESVKAIKLKILDMFLDPECTKQDDNWRKDLSTMSRFCGEAGNMCLRDLYNYFSMPKEDRISSKDLYNAMYHKTKLLHPELPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINHRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 324 - RNA, 176 nt, synthetic construct
  source 1..176: mol_type = other RNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaccgctgtcgcatcttgcgtaagcgcgtggattgaaac

SEQ ID NO: 325 - RNA, 159 nt, synthetic construct
  source 1..159: mol_type = other RNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacaaggtaagcgcgtggattgaaac

SEQ ID NO: 326 - RNA, 172 nt, synthetic construct
  source 1..172: mol_type = other RNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaagtcgcatcttgcgtaagcgcgtggattgaaac

SEQ ID NO: 327 - RNA, 159 nt, synthetic construct
  source 1..159: mol_type = other RNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaataagcgcgtggattgaaac

SEQ ID NO: 328 - DNA, 193 bp, synthetic construct
  source 1..193: mol_type = other DNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacgaaccgctgtcgcatcttgcgtaagcgcgtggattgaaaccctgtcttgtttgtgagagg

SEQ ID NO: 329 - DNA, 176 bp, synthetic construct
  source 1..176: mol_type = other DNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacaaggtaagcgcgtggattgaaaccctgtcttgtttgtgagagg

SEQ ID NO: 330 - DNA, 192 bp, synthetic construct
  source 1..192: mol_type = other DNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaagtcgcatcttgcgtaagcgcgtggattgaaaccctgtcttgtttgtgagagg

SEQ ID NO: 331 - DNA, 189 bp, synthetic construct
  source 1..189: mol_type = other DNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacgaaagtcgcatcttgcgtaagcgcgtggattgaaaccctgtcttgtttgtgagagg

SEQ ID NO: 332 - DNA, 189 bp, synthetic construct
  source 1..189: mol_type = other DNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaacgaaagtcgcatcttgcgtaagcgcgtggattgaaaccctgtcttgtttgtgagagg

SEQ ID NO: 333 - DNA, 189 bp, synthetic construct
  source 1..189: mol_type = other DNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaacgcatcttgcgtaagcgcgtggattgaaaccctgtcttgtttgtgagagg

SEQ ID NO: 334 - RNA, 193 nt, synthetic construct
  source 1..193: mol_type = other RNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacgaaccgctgtcgcatcttgcgtaagcgcgtggattgaaaccctgtcttgtttgtgagagg

SEQ ID NO: 335 - RNA, 176 nt, synthetic construct
  source 1..176: mol_type = other RNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacaaggtaagcgcgtggattgaaaccctgtcttgtttgtgagagg

SEQ ID NO: 336 - RNA, 192 nt, synthetic construct
  source 1..192: mol_type = other RNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaagtcgcatcttgcgtaagcgcgtggattgaaaccctgtcttgtttgtgagagg

SEQ ID NO: 337 - RNA, 189 nt, synthetic construct
  source 1..189: mol_type = other RNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacgaaagtcgcatcttgcgtaagcgcgtggattgaaaccctgtcttgtttgtgagagg

SEQ ID NO: 338 - RNA, 189 nt, synthetic construct
  source 1..189: mol_type = other RNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaacgaaagtcgcatcttgcgtaagcgcgtggattgaaaccctgtcttgtttgtgagagg

SEQ ID NO: 339 - RNA, 189 nt, synthetic construct
  source 1..189: mol_type = other RNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaacgcatcttgcgtaagcgcgtggattgaaaccctgtcttgtttgtgagagg

SEQ ID NO: 340 - DNA, 193 bp, synthetic construct
  source 1..193: mol_type = other DNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacgaaccgctgtcgcatcttgcgtaagcgcgtggattgaaacggttgtgttgccctccctgt

SEQ ID NO: 341 - DNA, 176 bp, synthetic construct
  source 1..176: mol_type = other DNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacaaggtaagcgcgtggattgaaacggttgtgttgccctccctgt

SEQ ID NO: 342 - DNA, 192 bp, synthetic construct
  source 1..192: mol_type = other DNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaagtcgcatcttgcgtaagcgcgtggattgaaacggttgtgttgccctccctgt

SEQ ID NO: 343 - DNA, 189 bp, synthetic construct
  source 1..189: mol_type = other DNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacgaaagtcgcatcttgcgtaagcgcgtggattgaaacggttgtgttgccctccctgt

SEQ ID NO: 344 - DNA, 189 bp, synthetic construct
  source 1..189: mol_type = other DNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaacgaaagtcgcatcttgcgtaagcgcgtggattgaaacggttgtgttgccctccctgt

SEQ ID NO: 345 - DNA, 189 bp, synthetic construct
  source 1..189: mol_type = other DNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaacgcatcttgcgtaagcgcgtggattgaaacggttgtgttgccctccctgt

SEQ ID NO: 346 - RNA, 193 nt, synthetic construct
  source 1..193: mol_type = other RNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacgaaccgctgtcgcatcttgcgtaagcgcgtggattgaaacggttgtgttgccctccctgt

SEQ ID NO: 347 - RNA, 176 nt, synthetic construct
  source 1..176: mol_type = other RNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacaaggtaagcgcgtggattgaaacggttgtgttgccctccctgt

SEQ ID NO: 348 - RNA, 192 nt, synthetic construct
  source 1..192: mol_type = other RNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaagtcgcatcttgcgtaagcgcgtggattgaaacggttgtgttgccctccctgt

SEQ ID NO: 349 - RNA, 189 nt, synthetic construct
  source 1..189: mol_type = other RNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacgaaagtcgcatcttgcgtaagcgcgtggattgaaacggttgtgttgccctccctgt

SEQ ID NO: 350 - RNA, 189 nt, synthetic construct
  source 1..189: mol_type = other RNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaacgaaagtcgcatcttgcgtaagcgcgtggattgaaacggttgtgttgccctccctgt

SEQ ID NO: 351 - RNA, 189 nt, synthetic construct
  source 1..189: mol_type = other RNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaacgcatcttgcgtaagcgcgtggattgaaacggttgtgttgccctccctgt

SEQ ID NO: 352 - RNA, 209 nt, synthetic construct
  source 1..209: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacaagttcacggcgttccaggc

SEQ ID NO: 353 - RNA, 209 nt, synthetic construct
  source 1..209: mol_type = other RNA
atttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaacggttctgggatagcttggga

SEQ ID NO: 354 - RNA, 179 nt, synthetic construct
  source 1..179: mol_type = other RNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaataagcgcgtggattgaaacaagttcacggcgttccaggc

SEQ ID NO: 355 - RNA, 179 nt, synthetic construct
  source 1..179: mol_type = other RNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaataagcgcgtggattgaaacggttctgggatagcttggga

SEQ ID NO: 356 - DNA, 173 bp, synthetic construct
  source 1..173: mol_type = other DNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacgaaccgctgtcgcatcttgcgtaagcgcgtggattgaaac

SEQ ID NO: 357 - DNA, 156 bp, synthetic construct
  source 1..156: mol_type = other DNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacaaggtaagcgcgtggattgaaac

SEQ ID NO: 358 - DNA, 172 bp, synthetic construct
  source 1..172: mol_type = other DNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaaagtcgcatcttgcgtaagcgcgtggattgaaac

SEQ ID NO: 359 - DNA, 169 bp, synthetic construct
  source 1..169: mol_type = other DNA
gtttcgcgcgccagggcagttaggtgccctaaaagagcgaagtggccgagaaaggctaacgcttctctaacgctacggcgaccttggcgaaatgccatcaataccacgcggcccgaaagggttcgcgcgaaacgaaagtcgcatcttgcgtaagcgcgtggattgaaac

SEQ ID NO: 360 - DNA, 169 bp, synthetic construct
  source 1..169: mol_type = other DNA
gtttcgcgcgccagggcagttaggtgccctaaaagagcgaagtggccgaaaaaggctaacgcttctctaacgctacggcgaccttggcgaaatgccatcaataccacgcggcccgaaagggttcgcgcgaaacgaaagtcgcatcttgcgtaagcgcgtggattgaaac

SEQ ID NO: 361 - DNA, 169 bp, synthetic construct
  source 1..169: mol_type = other DNA
gtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaacgaacgcatcttgcgtaagcgcgtggattgaaac

SEQ ID NO: 362 - protein, 497 aa, synthetic construct
MGESVKAIKLKILDMFLDPECTKQDDNWRKDLSEMSRFCGEAGNSCLRDLYNYFSMPKEDRISSKDLYNAMYHKTKLLHPELPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

(19) United States
(12) Patent Application Publication
(10) Pub. No.: US 2026/0218147 A1
(43) Pub. Date: Jul. 30, 2026

(54) CAS ENDONUCLEASE AND GUIDE RNA VARIANTS WITH IMPROVED EFFICIENCY

(71) Applicant: PIONEER HI-BRED INTERNATIONAL, INC., JOHNSTON, IA (US)

(72) Inventors: ELIZABETH SOMMERS VAN GINKLE, DES MOINES, IA (US); JOSHUA K. YOUNG, JOHNSTON, IA (US)

(73) Assignee: PIONEER HI-BRED INTERNATIONAL, INC., JOHNSTON, IA (US)

(21) Appl. No.: 18/874,795

(22) PCT Filed: Jun. 13, 2023
(86) PCT No.: PCT/US2023/068329

§ 371 Date: Dec. 13, 2024

Related U.S. Application Data
(60) Provisional application No. 63/352,100, filed on Jun. 14, 2022.
(60) Provisional application No. 63/378,897, filed on Oct. 10, 2022.
(60) Provisional application No. 63/481,458, filed on Jan. 25, 2023.
(60) Provisional application No. 63/497,314, filed on Apr. 20, 2023.

(51) Int. Cl. C12N 9/22; C12N 15/11; C12N 15/82; C12N 15/90

(52) U.S. Cl. CPC C12N 9/224; C12N 15/11; C12N 15/8213; C12N 15/905; C12N 15/907; C12N 2310/20

(57) ABSTRACT

Compositions and methods are provided for genome modification of a target sequence in the genome of a cell, using novel engineered Cas polypeptides. The engineered Cas polypeptides and related methods, systems and compositions disclosed herein can be used in a guide polynucleotide/endonuclease system to modify or alter target sequences within the genome of a cell or organism. Also provided are novel effectors and endonuclease systems and elements comprising such systems, such as guide polynucleotide/endonuclease systems comprising an endonuclease. Compositions and methods are also provided for guide polynucleotide/endonuclease systems comprising at least one endonuclease, optionally covalently or non-covalently linked to, or assembled with, at least one additional protein subunit, and for compositions and methods for direct delivery of endonucleases as ribonucleotide proteins.

### CROSS REFERENCE TO RELATED APPLICATIONS

This application claims the benefit of U.S. Provisional No. 63/352,100, filed Jun. 14, 2022; U.S. Provisional No. 63/378,897, filed Oct. 10, 2022; U.S. Provisional No. 63/481,458, filed Jan. 25, 2023; and U.S. Provisional No. 63/497,314, filed Apr. 20, 2023 the disclosure of each of which is incorporated by reference herein in its entirety.

### REFERENCE TO SEQUENCE LISTING SUBMITTED ELECTRONICALLY

The official copy of the sequence listing is submitted electronically herewith as an xml file named 9213-WO-PCT_ST26v2 created Jun. 13, 2023 and having a size of 422,257 bytes. The sequence listing comprised in this xml file is part of the specification and is herein incorporated by reference in its entirety.

### FIELD

The disclosure relates to the field of molecular biology, in particular to compositions of novel RNA-guided Cas endonuclease systems, and compositions and methods for editing or modifying the genome of a cell.

### BACKGROUND

Recombinant DNA technology has made it possible to insert DNA sequences at targeted genomic locations and/or modify specific endogenous chromosomal sequences. Site-specific integration techniques, which employ site-specific recombination systems, as well as other types of recombination technologies, have been used to generate targeted insertions of genes of interest in a variety of organism. Genome-editing techniques such as designer zinc finger nucleases (ZFNs), transcription activator-like effector nucleases (TALENs), or homing meganucleases, are available for producing targeted genome perturbations, but these systems tend to have low specificity and employ designed nucleases that need to be redesigned for each target site, which renders them costly and time-consuming to prepare.

Newer technologies utilizing archaeal or bacterial adaptive immunity systems have been identified, called CRISPR (Clustered Regularly Interspaced Short Palindromic Repeats), which comprise different domains of effector proteins that encompass a variety of activities (DNA recognition, binding, and optionally cleavage).

Despite the identification and characterization of some of these systems, there remains a need for engineering novel effectors and systems, as well as demonstrating activity in eukaryotes, particularly animals and plants, to effect editing of endogenous and previously introduced heterologous polynucleotides.

Herein are described novel engineered Cas polypeptides and endonucleases, and methods and compositions for use thereof.

### SUMMARY

Disclosed herein are compositions of novel engineered Cas polypeptides and methods of use thereof. These Cas polypeptides are capable of being guided by a guide polynucleotide to target double-stranded DNA in a PAM-dependent fashion. In some embodiments, the engineered Cas polypeptides are active endonucleases capable of introducing a break at the target site of the target double-stranded DNA. In some embodiments, the Cas polypeptide comprises one or more mutations that render it incapable of double-strand cutting, but permit single-strand cutting. In some embodiments, the Cas polypeptide comprises one or more mutations that render it incapable of cleaving either or both strands of a double-stranded polynucleotide, but it retains the ability to bind to a target polynucleotide sequence.

In one aspect, a novel engineered Cas polypeptide is provided comprising at least one zinc-finger-like domain, at least one bridge-helix-like domain, a tri-split RuvC domain (comprising non-contiguous RuvC-I domain, RuvC-II domain, and RuvC-III domain), optionally comprising a heterologous polynucleotide. Also provided is a synthetic composition comprising the novel engineered Cas polypeptide or endonuclease. In many aspects of the disclosed compositions or methods, at least one component that has been optimized for expression in a eukaryotic cell, particularly a plant cell, a fungal cell, or an animal cell, is provided. In particular examples, the Cas polypeptide is fewer than about 500 amino acids.

In all aspects of the novel Cas polypeptide disclosed herein, the polypeptide has been engineered to include different amino acid sequence from a native effector protein obtained or derived from Syntrophomonas palmitatica.

In a first aspect, provided is an engineered Cas polypeptide, or a polynucleotide encoding the engineered Cas polypeptide, wherein said engineered Cas polypide comprises, when aligned to SEQ ID NO:14 and relative to the amino acid position numbers of SEQ ID NO:14, one or more of the following amino acids: Glutamate at position 34, Alanine at position 36, Serine at position 45, Aspartate at position 54, Serine at position 77, Glutamine at position 85. In some examples of this first aspect, the engineered Cas polypeptide further comprises, when aligned to SEQ ID NO:14 and relative to the amino acid position numbers of SEQ ID NO:14, at least one, at least two, at least three, at least four, at least five, at least six, or seven of the following relative to the amino acid position numbers of SEQ ID NO:14: Aspartate or Glutamate at position 38, Aspartate at position 79, Proline at position 120, Aspartate at position 149, Glycine at 226, Glycine at 230, Histidine at position 293, Serine at position 298, Phenylalanine at position 306, Glutamate at 329, Serine at position 313, Asparagine at position 325, Glutamate at position 327, Valine at position 338, Cysteine at position 376, Cysteine at position 379, Cysteine at position 395, Cysteine at position 398, Cysteine at position 406, Lysine or Arginine at position 409, Asparagine or Arginine at position 421, Proline at position 430, Arginine at position 467, or Proline at position 468.

In a second aspect, provided is an engineered Cas polypeptide, or a polynucleotide encoding the engineered Cas polypeptide, wherein said engineered Cas polypide comprises a sequence having at least 90%, at least 91%, at least 92%, at least 93%, at least 94%, at least 95%, at least 96%, at least 97%, at least 98%, or at least 99% amino acid sequence identity to SEQ ID NO:14 and, when aligned to SEQ ID NO:14 and relative to the amino acid position numbers of SEQ ID NO:14, one or more of the following amino acids: Glutamate at position 34, Alanine at position 36, Serine at position 45, Aspartate at position 54, Serine at position 77, Glutamine at position 85, and wherein the engineered Cas polypeptide is capable of site specifically binding a target site of a polynucleotide. In some examples of this second aspect, the engineered Cas polypeptide comprises a sequence having at least 90%, at least 91%, at least 92%, at least 93%, at least 94%, at least 95%, at least 96%, at least 97%, at least 98%, at least 99%, or 100% amino acid sequence identity to one of SEQID NOs:15-56, SEQID NO:59, SEQID NO:60, or SEQID NOs:309-323 or SEQID NO:362.

Additional examples of each of the foregoing first aspect or second aspect are provided, wherein the engineered Cas polypeptide or polynucleotide encoding the engineered Cas polypeptide, is provided such that the engineered Cas polypeptide further comprises, when aligned to SEQ ID NO:14 and relative to the amino acid position numbers of SEQ ID NO:14, at least one, at least two, at least three, at least four, at least five, at least six, or seven of the following amino acids: Aspartate or Glutamate at position 38, Aspartate at position 79, Proline at position 120, Aspartate at position 149, Glycine at 226, Glycine at 230, Histidine at position 293, Serine at position 298, Phenylalanine at position 306, Glutamate at 329, Serine at position 313, Asparagine at position 325, Glutamate at position 327, Valine at position 338, Cysteine at 376, Cysteine at 379, Cysteine at 395, Cysteine at 398, Cysteine at position 406, Lysine or Arginine at position 409, Asparagine or Arginine at position 421, Proline at position 430, Arginine at position 467, or Proline at position 468.

In yet further examples of each of the foregoing first aspect or second aspect, when aligned to SEQ ID NO:14 and relative to the amino acid position numbers of SEQ ID NO:14, the engineered Cas polypeptide does not comprise at least one of the following: Aspartate or Glutamate at position 38, Aspartate at position 79, Proline at position 120, Aspartate at position 149, Glycine at position 226, Glycine at position 230, Histidine at position 293, Serine at position 298, Phenylalanine at position 306, Glutamate at 329, Serine at position 313, Asparagine at position 325, Glutamate at position 327, Valine at position 338, Cysteine at position 376, Cysteine at position 379, Cysteine at 395, Cysteine at position 398, Cysteine at position 406, Lysine or Arginine at position 409, Asparagine or Arginine at position 421, Proline at position 430, Arginine at position 467, or Proline at position 468.

In particular embodiments of the foregoing examples of the first aspect or second aspect, the engineered Cas polypeptide comprises fewer than 500 amino acids in length. In particular embodiments of the foregoing first aspect or second aspect, the engineered Cas polypeptide is a Cas endonuclease.

Also provided are embodiments of each of the foregoing first aspect or second aspect, wherein the engineered Cas polypeptide comprises at least one zinc-finger-like domain, at least one bridge-helix-like domain, a tri-split RuvC domain (comprising non-contiguous RuvC-I domain, RuvC-II domain, and RuvC-III domain), and optionally comprising a heterologous polynucleotide.

In a third aspect, a synthetic composition is provided comprising a CRISPR-Cas polypeptide, or a polynucleotide encoding the CRISPR-Cas polypeptide, wherein the CRISPR-Cas polypeptide comprises a sequence sharing at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 250, between 250 and 300, at least 300, between 300 and 350, at least 350, between 350 and 400, at least 400, or greater than 400 contiguous amino acids of a sequence selected from the group consisting of SEQ ID NOs:15-47, SEQ ID NOs:49-56, SEQID NO:59, SEQID NO:60, SEQID NOs:309-323 and SEQID NO:362.

Any of the methods or compositions herein may further comprise a heterologous polynucleotide. The heterologous polynucleotide may be selected from the group consisting of: a noncoding regulatory expression element such as a promoter, intron, enhancer, or terminator; a donor polynucleotide; a polynucleotide modification template, optionally comprising at least one nucleotide modification as compared to the sequence of a polynucleotide in a cell; a transgene; a guide RNA; a guide DNA; a guide RNA-DNA hybrid; an endonuclease; a nuclear localization signal; and a cell transit peptide.

In another aspect, methods are provided for using any of the compositions disclosed herein. In some embodiments, methods are provided that included the disclosed Cas polypeptide or endonuclease (i.e., any of the first aspect, or second aspect, or third aspect embodiments) binding to a target sequence of a polynucleotide, for example in the genome of a cell or in vitro. In some embodiments, the disclosed Cas polypeptide or endonuclease forms a complex with a guide polynucleotide, for example a guide RNA (gRNA) which can be a single guide RNA (sgRNA). In some embodiments, the complex recognizes, binds to, and optionally creates a nick (one strand) or a break (two strands) in the polynucleotide at or near the target sequence. In some embodiments, the nick or break is repaired via Non-Homologous End Joining (NHEJ). In some embodiments, the nick or break is repaired via Homology-Directed Repair (HDR) or via Homologous Recombination (HR), with a polynucleotide modification template or a donor DNA molecule.

In any aspect, the engineered Cas polypeptide or endonuclease may be used to bind, nick and/or break a polynucleotide comprising a target sequence at a temperature of less than about 45 degrees Celsius, e.g., a temperature of about 40 degrees Celsius or less, about 37 degrees Celsius or less, about 35 degrees Celsius or less, about 30 degrees Celsius or less, about 28 degrees Celsius or less, or about 25 degrees Celsius or less. Thus, also provided is a method that includes contacting a polynucleotide with any engineered Cas polypeptide or endonuclease disclosed herein (e.g., any Cas polypeptide of the first, second, or third aspect disclosed herein) and creating a break in the polynucleotide at a temperature of less than about 45 degrees Celsius e.g., a temperature of about 40 degrees Celsius or less, about 35 degrees Celsius or less, about 30 degrees Celsius or less, about 28 degrees Celsius or less, or about 25 degrees Celsius or less. This break can be used to generate a targeted modification or altered target site (such as a base edit, deletion, or insertion) in the polynucleotide. Thus in some examples, the engineered Cas polypeptide or endonuclease of the first aspect, second aspect, or third aspect disclosed herein provides greater activity (binding, targeting efficiency, and/or endonuclease activity) than wild type Cas-alpha 10 endonuclease SEQ ID NO:48 or its base variant SEQ ID NO:14 at a temperature of less than about 45 degrees Celsius e.g., a temperature of about 40 degrees Celsius or less, about 35 degrees Celsius or less, about 30 degrees Celsius or less, about 28 degrees Celsius or less, or about 25 degrees Celsius or less.

The novel engineered Cas endonucleases described herein are capable of creating a double-strand break in, or adjacent to, a target polynucleotide that comprises an appropriate PAM, and to which it is directed by a guide polynucleotide, in any prokaryotic or eukaryotic cell. In some cases, the cell is a plant cell or an animal cell or a fungal cell. In some cases, a plant cell is selected from the group consisting of: maize, soybean, cotton, wheat, canola, oilseed rape, sorghum, rice, rye, barley, millet, oats, sugarcane, turfgrass, switchgrass, alfalfa, sunflower, tobacco, peanut, potato, tobacco, Arabidopsis, safflower, and tomato.

In another aspect, the engineered Cas polypeptide described herein comprises one or mutations that provide a nuclease inactivated or dead Cas polypeptide. For example, the engineered Cas polypeptide disclosed herein can be altered such that, when aligned with SEQ ID NO:14 or 48 and relative to the amino acid position numbers of SEQ ID NO:14 or 48, the engineered Cas polypeptide disclosed herein includes an alanine at position 228; an alanine at relative position 327, or an alanine at position 434. The disclosed inactivated engineered Cas polypeptide can be linked to an effector or effector protein, which can be a molecule that recognizes, binds to, and/or cleaves or nicks a polynucleotide target. The disclosed inactivated engineered Cas polypeptide can be linked to a base editing molecule, e.g., a deaminase, for targeted base editing. The disclosed inactivated engineered Cas polypeptide optionally linked to an effector or effector protein, can be used for targeted deliver of an effector molecule at a temperature of about 45 degrees Celsius or less, about 40 degrees Celsius or less, about 37 degrees Celsius or less, about 35 degrees Celsius or less, about 30 degrees Celsius or less, or about 25 degrees Celsius or less.

In still another aspect, provided is a method of altering a target Cas polypeptide; the method comprising modifying the target Cas polypeptide to include one or more amino acid changes such that, when aligned with SEQ ID NO:14 or 48 and relative to the amino acid position numbers of SEQ ID NO:14 or 48, the target Cas polypeptide comprises one or more of the following amino acid alterations: Glutamate at position 34, Alanine at position 36, Serine at position 45, Aspartate at position 54, Serine at position 77, and Glutamine at position 85, wherein the engineered Cas polypeptide is capable of site specifically binding a target site of a polynucleotide, and thereby producing an altered Cas polypeptide. Optionally, in some examples of this aspect, the target Cas polypeptide is further modified such that, when aligned with SEQ ID NO:14 or 48 and relative to the amino acid position numbers of SEQ ID NO:14 or 48, the target Cas polypeptide comprises one of the following amino acid alterations: Aspartate or Glutamate at position 38, Aspartate at position 79, Proline at position 120, Aspartate at position 149, Glycine at 226, Glycine at 230, Histidine at position 293, Serine at position 298, Phenylalanine at position 306, Glutamate at 329, Serine at position 313, Asparagine at position 325, Glutamate at position 327, Valine at position 338, Cysteine at 376, Cysteine at 379, Cysteine at 395, Cysteine at 398, Cysteine at position 406, Lysine or Arginine at position 409, Asparagine or Arginine at position 421, Proline at position 430, Arginine at position 467, or Proline at position 468.

For example, in certain examples of the foregoing aspect, the method comprises creating an alignment of the target Cas polypeptide (e.g. a Cas polypeptide of less than 500 amino acids in length) to SEQ ID NO:14 or 48, identifying the target Cas polypeptide amino acid residue(s) corresponding to one or more of amino acid positions 34, 36, 45, 54, 77, and 85 of SEQ ID NO:14 or 48, and modifying the target Cas polypeptide amino acid residue(s) to one or more of the following: Glutamate at position 34, Alanine at position 36, Serine at position 45, Aspartate at position 54, Serine at position 77, and Glutamine at position 85, respectively. Optionally, the method further comprises identifying the target Cas polypeptide amino acid residue(s) corresponding to one or more of amino acid positions 38, 79, 120, 149, 226, 230, 293, 298, 306, 329, 313, 325, 327, 338, 376, 379. 395, 398, 406, 409, 421, 430, 467, and 468 of SEQ ID NO:14 or 48 and modifying the target Cas polypeptide amino acid residue(s) to one or more of the following: Aspartate or Glutamate at position 38, Aspartate at position 79, Proline at position 120, Aspartate at position 149, Glycine at 226, Glycine at 230, Histidine at position 293, Serine at position 298, Phenylalanine at position 306, Glutamate at 329, Serine at position 313, Asparagine at position 325, Glutamate at position 327, Valine at position 338, Cysteine at 376, Cysteine at 379, Cysteine at 395, Cysteine at 398, Cysteine at position 406, Lysine or Arginine at position 409, Asparagine or Arginine at position 421, Proline at position 430, Arginine at position 467, or Proline at position 468, respectively. In these methods the target Cas polypeptide can (i) have at least 90%, at least 91%, at least 92%, at least 93%, at least 94%, at least 95%, at least 96%, at least 97%, at least 98%, or at least 99% amino acid sequence identity to SEQ ID NO:14, (ii) be fewer than 500 amino acids in length, or (iii) both of (i) and (ii). In particular examples the modified target Cas polypeptide provide greater activity (binding, targeting efficiency, and/or endonuclease activity) than the unmodified target Cas polypeptide at a temperature of less than about 45 degrees Celsius e.g., a temperature of about 40 degrees Celsius or less, about 35 degrees Celsius or less, about 30 degrees Celsius or less, about 28 degrees Celsius or less, or about 25 degrees Celsius or less.

This method of altering a target Cas polypeptide can include, for example, modifying a nucleic acid sequence encoding the target Cas polypeptide to include one or more altered codons that, when aligned with SEQ ID NO:14 or 48 and relative to the amino acid position numbers of SEQ ID NO:14 or 48, encodes one or more of the following amino acid alterations: Glutamate at position 34, Alanine at position 36, Serine at position 45, Aspartate at position 54, Serine at position 77, and Glutamine at position 85, and thereby creating an altered coding sequence that encodes the altered Cas polypeptide. Optionally, the method includes altering the nucleic acid sequence encoding the target Cas polypeptide to also include one or more additional altered codons that, when aligned with SEQ ID NO:14 or 48 and relative to the amino acid position numbers of SEQ ID NO:14 or 48, encodes one or more of the following amino acid alterations: Aspartate or Glutamate at position 38, Aspartate at position 79, Proline at position 120, Aspartate at position 149, Glycine at 226, Glycine at 230, Histidine at position 293, Serine at position 298, Phenylalanine at position 306, Glutamate at 329, Serine at position 313, Asparagine at position 325, Glutamate at position 327, Valine at position 338, Cysteine at 376, Cysteine at 379, Cysteine at 395, Cysteine at 398, Cysteine at position 406, Lysine or Arginine at position 409, Asparagine or Arginine at position 421, Proline at position 430, Arginine at position 467, or Proline at position 468.

Also provided herein is a system comprising a Cas polypeptide and an engineered guide RNA (egRNA), wherein the egRNA is based on a template guide RNA sequence (sgRNA) that comprises a Cas polypeptide recognition domain and wherein the egRNA comprises (i) a deletion, substitution or an insertion at one or more of nucleotide positions 1-3, 3-5, 5-7, 7-9, 59-61, 61-63, 63-65, 147-149, 149-151, 151-153, 153-155, 157-159, 159-161, 163-165, or 167-169 of the sgRNA Cas polypeptide recognition domain or any combination thereof, (ii) the sequence of one of SEQ ID NOs: 62-156, SEQ ID NOs: 192-207, SEQ ID NOs: 260-284, SEQ ID NOs: 324-327, or SEQ ID NOs: 346-355; or (iii) an aptamer inserted at Position 1, 3, 4, 5, 6, 7, 9, 10 or 11 of the sgRNA Cas polypeptide recognition domain. Thus the disclosed egRNA can comprise a deletion, substitution or an insertion at (i) positions 3-5, 5-7, 7-9, 59-61, 61-63, 63-65, 149-151, 151-153, 153-155, 157-159, 163-165, or 167-169 or any combination thereof; (ii) positions 1-9, or 146-153, or (iii) positions 1-9, 6-63, 61-63, 146-153, 157-159, 158-170, 167-169 or any combination thereof. Such engineered guide (egRNA) and their functionality (effect on target DNA cleavage efficiency) are shown in Table 6, Table 7, Table 8, Table 9, Table 10, Table 11, and Table 12 herein.

In some examples of any of the foregoing system comprising a Cas polypeptide and an engineered guide RNA (egRNA), the Cas polypeptide is one of the engineered Cas polypeptides disclosed herein, e.g., the engineered Cas polypide that (i) comprises, when aligned to SEQ ID NO:14, relative to the amino acid position numbers of SEQ ID NO:14, one or more of the following amino acid alterations: Glutamate at position 34, Alanine at position 36, Serine at position 45, Aspartate at position 54, Serine at position 77, Glutamine at position 85 (ii) comprises a sequence having at least 90%, at least 91%, at least 92%, at least 93%, at least 94%, at least 95%, at least 96%, at least 97%, at least 98%, or at least 99% amino acid sequence identity to SEQ ID NO:14 and, when aligned to SEQ ID NO:14, relative to the amino acid position numbers of SEQ ID NO:14, one or more of the following amino acids: Glutamate at position 34, Alanine at position 36, Serine at position 45, Aspartate at position 54, Serine at position 77, Glutamine at position 85, and wherein the engineered Cas polypeptide is capable of site specifically binding a target site of a polynucleotide, (iii) comprises a sequence having at least 90%, at least 91%, at least 92%, at least 93%, at least 94%, at least 95%, at least 96%, at least 97%, at least 98%, at least 99%, or 100% amino acid sequence identity to one of SEQID NOs:15-56, SEQID NO:59, SEQID NO:60, SEQID NOs:309-323 or SEQID NO:362. Particular examples of a system comprising an engineered Cas polypeptide and an engineered guide RNA (egRNA) are disclosed herein in each of Table 6, Table 7, Table 8, Table 9, Table 10, Table 11 and Table 12 herein.

### BRIEF DESCRIPTION OF THE DRAWINGS AND THE SEQUENCE LISTING

The disclosure can be more fully understood from the following detailed description and the accompanying drawings and Sequence Listing, which form a part of this application.

FIG. 1 depicts a Cas endonuclease variant yeast expression vector, wherein: 1=ROX3 promoter (SEQ ID NO:1); 2=Yeast optimized Cas endonuclease gene (SEQ ID NO:2); 3=Sequence encoding SV40 NLS (SEQ ID NO:3); 4=CYC1 terminator (SEQ ID NO:4); 5=SNR52 promoter (SEQ ID NO:5); 6=Sequence encoding Cas endonuclease sgRNA (Cas endonuclease recognition domain) (SEQ ID NO:6); 7=Sequence encoding Cas-alpha 10 sgRNA ADE2 targeting domain (SEQ ID NO:7); 8=Sequence encoding Cas-alpha 10 sgRNA CAN1 targeting domain (SEQ ID NO:8); 9=Sequence encoding Cas-alpha 10 sgRNA targeting ADE2 (SEQ ID NO:9); 10=Sequence encoding Cas-alpha 10 sgRNA targeting CAN1 (SEQ ID NO:10).

FIG. 2 is a schema for target cleavage detection in S. cerevisiae, wherein target cleavage and cellular repair results in the formation of a non-functional ade2 gene that results in adenine auxotrophy and a switch from a white to a red/pink cellular phenotype. This color switch allows for identification of phenotypic differences and can be used to select cells expressing a Cas endonuclease variant and/or associated guide RNA with improved targeted DSB activity. Colonies with functional ade2 gene are white. Colonies with non-functional ade2 gene are red. Colony that are mottled red and white indicate multiple sectors containing a non-functional ade2 gene. Colony with a single sector containing a non-functional ade2 gene are white with a red section.

FIG. 3 is a schema for target cleavage detection in S. cerevisiae, wherein target cleavage and cellular non-functional repair of the CAN1 gene results in resistance against L-canavanine, thereby allowing for positive selection of Cas endonuclease variants with improved double stranded breaking activity.

FIG. 4 is the polypeptide sequence for a Cas endonuclease (SEQ ID NO:14) which includes several modifications that enable its function at lower temperatures, and which was further altered in the Examples described herein. Key catalytic residues are shown in bold underlined font. Zinc finger domains are indicated by a dashed underline.

FIG. 5 is the polypeptide sequence for an engineered Cas polypeptide (SEQ ID NO:60) having the improved activity shown in Examples herein. Key catalytic residues are shown in bold underlined font. Zinc finger domains are indicated by a dashed underline. Double underlined letters show further altered residues relative to the base Cas endonuclease (SEQ ID NO:14) used herein.

FIG. 6 shows two schematic diagrams of human-optimized expression cassettes: the upper shows a cassette for Cas-alpha nuclease and the lower shows is a cassette for expression of Cas-alpha sgRNA.

FIG. 7 shows an engineered sgRNA comprising MS2 recognition sequence incorporated at the 5′ end, the apex of stem-loops, and within the 5′-GAAA-3′ linker connecting the tracrRNA and crRNA of the sgRNA; FIG. 7 also shows an example of Positions 1-12, at which aptamers or Cas6 sequences where inserted as described in Table 9 herein.

FIG. 8 shows two schematic diagrams of Zea mays optimized expression cassettes: the upper shows cassette for Cas-alpha nuclease and the lower shows a cassette for expression of Cas-alpha sgRNA.

FIG. 9 is a graph showing the difference of cleavage of between base variant Cas endonuclease SEQ ID NO:14 and wild type Cas endonuclease SEQ ID NO:157 at different temperatures.

FIG. 10 is a graph showing the difference of cleavage of between variant Cas endonucleases SEQ ID NO:14 and SEQ ID NO:48 at different temperatures.

FIG. 11 is a graph showing the difference of cleavage of between variant Cas endonucleases SEQ ID NO:14 and SEQ ID NO: 362 at different temperatures, the graph shows the significantly improved cleavage activity of SEQID NO:362 at 25° C.

Table 1 provides a description of sequences referenced herein. The isolated polynucleotides, nucleic acids, and nucleotide sequences disclosed herein additionally encompass all complementary forms (e.g., the reverse complement) of each sequence disclosed for such a construct. The sequence descriptions and sequence listing attached hereto comply with the rules governing nucleotide and amino acid sequence disclosures in patent applications as set forth in 37 C.F.R. §§ 1.821-1.835.

TABLE 1

| Seq Listing No. | Description |
|---|---|
| SEQ ID NO: 1 | Saccharomyces cerevisiae DNA ROX3 |
| | promoter sequence |
| SEQ ID NO: 2 | artificial yeast optimized Cas |
| | endonuclease gene sequence |
| SEQ ID NO: 3 | artificial yeast optimized sequence |
| | encoding SV40 NLS |
| SEQ ID NO: 4 | CYC1 terminator sequence |
| SEQ ID NO: 5 | Saccharomyces cerevisiae SNR52 |
| | promoter sequence |
| SEQ ID NO: 6 | artificial Sequence encoding Cas |
| | endonuclease sgRNA (Cas endonuclease |
| | recognition domain) sequence |
| SEQ ID NO: 7 | Saccharomyces cerevisiae DNA Sequence |
| | encoding Cas-alpha 10 sgRNA ADE2 |
| | targeting domain |
| SEQ ID NO: 8 | Saccharomyces cerevisiae DNA Sequence |
| | encoding Cas-alpha 10 sgRNA CAN1 |
| | targeting domain |
| SEQ ID NO: 9 | Artificial Sequence DNA encoding |
| | Cas-alpha 10 sgRNA targeting ADE2 |
| SEQ ID NO: 10 | Artificial Sequence DNA encoding |
| | Cas-alpha 10 sgRNA targeting CAN1 |
| SEQ ID NO: 11 | Hepatitis Delta Virus (HDV) sequence |
| | encoding HDV ribozyme |
| SEQ ID NO: 12 | Saccharomyces cerevisiae DNA SUP4 |
| | terminator sequence |
| SEQ ID NO: 13 | Simian virus 40 PRT SV40 NLS protein sequence |
| SEQ ID NO: 14 | Base Variant Cas-alpha protein sequence |
| SEQ ID NO: 15 | Cas-alpha 10 T34E variant protein sequence |
| SEQ ID NO: 16 | Cas-alpha 10 S36A variant protein sequence |
| SEQ ID NO: 17 | Cas-alpha 10 M45S variant protein sequence |
| SEQ ID NO: 18 | Cas-alpha 10 F54D variant protein sequence |
| SEQ ID NO: 19 | Cas-alpha 10 L77Svariant protein sequence |
| SEQ ID NO: 20 | Cas-alpha 10 K85Q variant protein sequence |
| SEQ ID NO: 21 | Cas-alpha 10 K155N variant protein sequence |
| SEQ ID NO: 22 | Cas-alpha 10 K198C variant protein sequence |
| SEQ ID NO: 23 | Cas-alpha 10 H215T variant protein sequence |
| SEQ ID NO: 24 | Cas-alpha 10 G320E variant protein sequence |
| SEQ ID NO: 25 | Cas-alpha 10 L455A variant protein sequence |
| SEQ ID NO: 26 | Cas-alpha 10 1464T variant protein sequence |
| SEQ ID NO: 27 | Cas-alpha 10 T465E variant protein sequence |
| SEQ ID NO: 28 | Cas-alpha 10 G470D variant protein sequence |
| SEQ ID NO: 29 | Cas-alpha 10 L488E variant protein sequence |
| SEQ ID NO: 30 | Cas-alpha 10 A496N variant |
| SEQ ID NO: 31 | Cas-alpha 10 T34E + F54D variant |
| | protein sequence |
| SEQ ID NO: 32 | Cas-alpha 10 S36A + F54D variant |
| | protein sequence |
| SEQ ID NO: 33 | Cas-alpha 10 M45S + F54D variant |
| | protein sequence |
| SEQ ID NO: 34 | Cas-alpha 10 F54D + K85Q variant |
| | protein sequence |
| SEQ ID NO: 35 | Cas-alpha 10 F54D + G470D variant |
| | protein sequence |
| SEQ ID NO: 36 | Cas-alpha 10 F54D + L488E variant |
| | protein sequence |
| SEQ ID NO: 37 | Cas-alpha 10 T34E + F54D + K85Q |
| | variant a.a. sequence |
| SEQ ID NO: 38 | Cas-alpha 10 S36A + F54D + K85Q |
| | variant a.a. sequence |
| SEQ ID NO: 39 | Cas-alpha 10 M45S + F54D + K85Q |
| | variant a.a. sequence |
| SEQ ID NO: 40 | Cas-alpha 10 F54D + K85Q + G470D |
| | variant a.a. sequence |
| SEQ ID NO: 41 | Cas-alpha 10 F54D + K85Q + L488E |
| | variant a.a. sequence |
| SEQ ID NO: 42 | Cas-alpha 10 T34E + F54D + K85Q + G470D |
| | variant a.a. sequence |
| SEQ ID NO: 43 | Cas-alpha 10 S36A + F54D + K85Q + G470D |
| | variant a.a. sequence |
| SEQ ID NO: 44 | Cas-alpha 10 M45S + F54D + K85Q + G470D |
| | a.a. protein sequence |
| SEQ ID NO: 45 | Cas-alpha T34E + 10 F54D + K85Q + T34E + |
| | L488E variant a.a. sequence |
| SEQ ID NO: 46 | Cas-alpha S36A + 10 F54D + K85Q + L488E |
| | variant a.a. sequence |
| SEQ ID NO: 47 | Cas-alpha M45S + 10 F54D + K85Q + L488E |
| | variant a.a. sequence |
| SEQ ID NO: 48 | Cas-alpha 10 endonuclease variant protein |
| | sequence used as base sequence |
| | for further modifications and creation of |
| | additional variants described herein |
| SEQ ID NO: 49 | Cas-alpha 10 T34E + F54D + G81E + K85Q + |
| | G470D variant a.a. sequence |
| SEQ ID NO: 50 | Cas-alpha 10 M45S + F54D + G81E + K85Q + |
| | G470D variant a.a. sequence |
| SEQ ID NO: 51 | Cas-alpha T34E + M45S + F54D + K85Q + |
| | G470D variant a.a. sequence |
| SEQ ID NO: 52 | Cas-alpha 10 T34E + S36A + F54D + K85Q + |
| | G470D variant a.a. sequence |
| SEQ ID NO: 53 | Cas-alpha 10 T34E + S36A + F54D + G81E + |
| | K85Q + G470D variant a.a. sequence |
| SEQ ID NO: 54 | Cas-alpha 10T34E + M45S + F54D + G81E + |
| | K85Q + G470D variant a.a. sequence |
| SEQ ID NO: 55 | Cas-alpha 10 T34E + S36A + M45S + F54D + |
| | G81E + K85Q + G470D variant a.a. |
| | sequence |
| SEQ ID NO: 56 | Cas-alpha 10 T34E + S36A + M45S + F54D + |
| | K85Q + G470D variant a.a. sequence |
| SEQ ID NO: 57 | DE2-CR2 target DNA sequence |
| SEQ ID NO: 58 | DE2-CR1 target DNA sequence |
| SEQ ID NO: 59 | Cas-alpha 10 M45S + F54D + G81E + G470D |
| | variant a.a. sequence |
| SEQ ID NO: 60 | Cas-alpha 10 T34E + M45S + F54D + G81E + |
| | G470D variant a.a. sequence |
| SEQ ID NO: 61 | Cas-alpha 10 sgRNA (Cas-alpha |
| | recognition domain) |
| SEQ ID NO: 62 | Cas-alpha 10 sgRNA targeting ADE2-CR2 |
| SEQ ID NO: 63 | Cas-alpha 10 sgRNA targeting ADE2-CR2 |
| | with positions 1-3 deleted |
| SEQ ID NO: 64 | Cas-alpha 10 sgRNA targeting ADE2-CR2 |
| | with positions 3-5 deleted |
| SEQ ID NO: 65 | Cas-alpha 10 sgRNA targeting ADE2-CR2 |
| | with positions 5-7 deleted |
| SEQ ID NO: 66 | Cas-alpha 10 sgRNA targeting ADE2-CR2 |
| | with positions 7-9 deleted |
| SEQ ID NO: 67 | Cas-alpha 10 sgRNA targeting ADE2-CR2 |
| | with positions 9-11 deleted |
| SEQ ID NO: 68 | Cas-alpha 10 sgRNA targeting ADE2-CR2 |
| | with positions 11-13 deleted |
| SEQ ID NO: 69 | Cas-alpha 10 sgRNA targeting ADE2-CR2 |
| | with positions 13-15 deleted |
| SEQ ID NO: 70 | Cas-alpha 10 sgRNA targeting ADE2-CR2 |
| | with positions 15-17 deleted |
| SEQ ID NO: 71 | Cas-alpha 10 sgRNA targeting ADE2-CR2 |
| | with positions 17-19 deleted |
| SEQ ID NO: 72 | Cas-alpha 10 sgRNA targeting ADE2-CR2 |
| | with positions 19-21 deleted |
| SEQ ID NO: 73 | Cas-alpha 10 sgRNA targeting ADE2-CR2 |
| | with positions 21-23 deleted |
| SEQ ID NO: 74 | Cas-alpha 10 sgRNA targeting ADE2-CR2 |
| | with positions 23-25 deleted |
| SEQ ID NO: 75 | Cas-alpha 10 sgRNA targeting ADE2-CR2 |
| | with positions 25-27 deleted |
| SEQ ID NO: 76 | Cas-alpha 10 sgRNA targeting ADE2-CR2 |
| | with positions 27-29 deleted |
| SEQ ID NO: 77 | Cas-alpha 10 sgRNA targeting ADE2-CR2 |
| | with positions 29-31 deleted |
| SEQ ID NO: 78 | Cas-alpha 10 sgRNA targeting ADE2-CR2 |
| | with positions 31-33 deleted |
| SEQ ID NO: 79 | Cas-alpha 10 sgRNA targeting ADE2-CR2 |
| | with positions 33-35 deleted |
| SEQ ID NO: 80 | Cas-alpha 10 sgRNA targeting ADE2-CR2 |
| | with positions 35-37 deleted |
| SEQ ID NO: 81 | Cas-alpha 10 sgRNA targeting ADE2-CR2 |
| | with positions 37-39 deleted |
| SEQ ID NO: 82 | Cas-alpha 10 sgRNA targeting ADE2-CR2 |
| | with positions 39-41 deleted |
| SEQ ID NO: 83 | Cas-alpha 10 sgRNA targeting ADE2-CR2 |
| | with positions 41-43 deleted |
| SEQ ID NO: 84 | Cas-alpha 10 sgRNA targeting ADE2-CR2 |
| | with positions 43-45 deleted |
| SEQ ID NO: 85 | Cas-alpha 10 sgRNA targeting ADE2-CR2 |
| | with positions 45-47 deleted |
| SEQ ID NO: 86 | Cas-alpha 10 sgRNA targeting ADE2-CR2 |
| | with positions 47-49 deleted |
| SEQ ID NO: 87 | Cas-alpha 10 sgRNA targeting ADE2-CR2 |
| | with positions 49-51 deleted |
| SEQ ID NO: 88 | Cas-alpha 10 sgRNA targeting ADE2-CR2 |
| | with positions 51-53 deleted |
| SEQ ID NO: 89 | Cas-alpha 10 sgRNA targeting ADE2-CR2 |
| | with positions 53-55 deleted |
| SEQ ID NO: 90 | Cas-alpha 10 sgRNA targeting ADE2-CR2 |
| | with positions 55-57 deleted |
| SEQ ID NO: 91 | Cas-alpha 10 sgRNA targeting ADE2-CR2 |
| | with positions 57-59 deleted |
| SEQ ID NO: 92 | Cas-alpha 10 sgRNA targeting ADE2-CR2 |
| | with positions 59-61 deleted |
| SEQ ID NO: 93 | Cas-alpha 10 sgRNA targeting ADE2-CR2 |
| | with positions 61-63 deleted |
| SEQ ID NO: 94 | Cas-alpha 10 sgRNA targeting ADE2-CR2 |
| | with positions 63-65 deleted |
| SEQ ID NO: 95 | Cas-alpha 10 sgRNA targeting ADE2-CR2 |
| | with positions 65-67 deleted |
| SEQ ID NO: 96 | Cas-alpha 10 sgRNA targeting ADE2-CR2 |
| | with positions 67-69 deleted |
| SEQ ID NO: 97 | Cas-alpha 10 sgRNA targeting ADE2-CR2 |
| | with positions 69-71 deleted |
| SEQ ID NO: 98 | Cas-alpha 10 sgRNA targeting ADE2-CR2 |
| | with positions 71-73 deleted |
| SEQ ID NO: 99 | Cas-alpha 10 sgRNA targeting ADE2-CR2 |
| | with positions 73-75 deleted |
| SEQ ID NO: 100 | Cas-alpha 10 sgRNA targeting ADE2-CR2 |
| | with positions 75-77 deleted |
| SEQ ID NO: 101 | Cas-alpha 10 sgRNA targeting ADE2-CR2 |
| | with positions 77-79 deleted |
| SEQ ID NO: 102 | Cas-alpha 10 sgRNA targeting ADE2-CR2 |
| | with positions 79-81 deleted |
| SEQ ID NO: 103 | Cas-alpha 10 sgRNA targeting ADE2-CR2 |
| | with positions 81-83 deleted |
| SEQ ID NO: 104 | Cas-alpha 10 sgRNA targeting ADE2-CR2 |
| | with positions 83-85 deleted |
| SEQ ID NO: 105 | Cas-alpha 10 sgRNA targeting ADE2-CR2 |
| | with positions 85-87 deleted |
| SEQ ID NO: 106 | Cas-alpha 10 sgRNA targeting ADE2-CR2 |
| | with positions 87-89 deleted |
| SEQ ID NO: 107 | Cas-alpha 10 sgRNA targeting ADE2-CR2 |
| | with positions 89-91 deleted |
| SEQ ID NO: 108 | Cas-alpha 10 sgRNA targeting ADE2-CR2 |
| | with positions 91-93 deleted |
| SEQ ID NO: 109 | Cas-alpha 10 sgRNA targeting ADE2-CR2 |
| | with positions 93-95 deleted |
| SEQ ID NO: 110 | Cas-alpha 10 sgRNA targeting ADE2-CR2 |
| | with positions 95-97 deleted |
| SEQ ID NO: 111 | Cas-alpha 10 sgRNA targeting ADE2-CR2 |
| | with positions 97-99 deleted |
| SEQ ID NO: 112 | Cas-alpha 10 sgRNA targeting ADE2-CR2 |
| | with positions 99-101 deleted |
| SEQ ID NO: 113 | Cas-alpha 10 sgRNA targeting ADE2-CR2 |
| | with positions 101-103 deleted |
| SEQ ID NO: 114 | Cas-alpha 10 sgRNA targeting ADE2-CR2 |
| | with positions 103-105 deleted |
| SEQ ID NO: 115 | Cas-alpha 10 sgRNA targeting ADE2-CR2 |
| | with positions 105-107 deleted |
| SEQ ID NO: 116 | Cas-alpha 10 sgRNA targeting ADE2-CR2 |
| | with positions 107-109 deleted |
| SEQ ID NO: 117 | Cas-alpha 10 sgRNA targeting ADE2-CR2 |
| | with positions 109-111 deleted |
| SEQ ID NO: 118 | Cas-alpha 10 sgRNA targeting ADE2-CR2 |
| | with positions 111-113 deleted |
| SEQ ID NO: 119 | Cas-alpha 10 sgRNA targeting ADE2-CR2 |
| | with positions 113-115 deleted |
| SEQ ID NO: 120 | Cas-alpha 10 sgRNA targeting ADE2-CR2 |
| | with positions 115-117 deleted |
| SEQ ID NO: 121 | Cas-alpha 10 sgRNA targeting ADE2-CR2 |
| | with positions 117-119 deleted |
| SEQ ID NO: 122 | Cas-alpha 10 sgRNA targeting ADE2-CR2 |
| | with positions 119-121 deleted |
| SEQ ID NO: 123 | Cas-alpha 10 sgRNA targeting ADE2-CR2 |
| | with positions 121-123 deleted |
| SEQ ID NO: 124 | Cas-alpha 10 sgRNA targeting ADE2-CR2 |
| | with positions 123-125 deleted |
| SEQ ID NO: 125 | Cas-alpha 10 sgRNA targeting ADE2-CR2 |
| | with positions 125-127 deleted |
| SEQ ID NO: 126 | Cas-alpha 10 sgRNA targeting ADE2-CR2 |
| | with positions 127-129 deleted |
| SEQ ID NO: 127 | Cas-alpha 10 sgRNA targeting ADE2-CR2 |
| | with positions 129-131 deleted |
| SEQ ID NO: 128 | Cas-alpha 10 sgRNA targeting ADE2-CR2 |
| | with positions 131-133 deleted |
| SEQ ID NO: 129 | Cas-alpha 10 sgRNA targeting ADE2-CR2 |
| | with positions 133-135 deleted |
| SEQ ID NO: 130 | Cas-alpha 10 sgRNA targeting ADE2-CR2 |
| | with positions 135-137 deleted |
| SEQ ID NO: 131 | Cas-alpha 10 sgRNA targeting ADE2-CR2 |
| | with positions 137-139 deleted |
| SEQ ID NO: 132 | Cas-alpha 10 sgRNA targeting ADE2-CR2 |
| | with positions 139-141 deleted |
| SEQ ID NO: 133 | Cas-alpha 10 sgRNA targeting ADE2-CR2 |
| | with positions 141-143 deleted |
| SEQ ID NO: 134 | Cas-alpha 10 sgRNA targeting ADE2-CR2 |
| | with positions 143-145 deleted |
| SEQ ID NO: 135 | Cas-alpha 10 sgRNA targeting ADE2-CR2 |
| | with positions 145-147 deleted |
| SEQ ID NO: 136 | Cas-alpha 10 sgRNA targeting ADE2-CR2 |
| | with positions 147-149 deleted |
| SEQ ID NO: 137 | Cas-alpha 10 sgRNA targeting ADE2-CR2 |
| | with positions 149-151 deleted |
| SEQ ID NO: 138 | Cas-alpha 10 sgRNA targeting ADE2-CR2 |
| | with positions 151-153 deleted |
| SEQ ID NO: 139 | Cas-alpha 10 sgRNA targeting ADE2-CR2 |
| | with positions 153-155 deleted |
| SEQ ID NO: 140 | Cas-alpha 10 sgRNA targeting ADE2-CR2 |
| | with positions 155-157 deleted |
| SEQ ID NO: 141 | Cas-alpha 10 sgRNA targeting ADE2-CR2 |
| | with positions 157-159 deleted |
| SEQ ID NO: 142 | Cas-alpha 10 sgRNA targeting ADE2-CR2 |
| | with positions 159-161 deleted |
| SEQ ID NO: 143 | Cas-alpha 10 sgRNA targeting ADE2-CR2 |
| | with positions 161-163 deleted |
| SEQ ID NO: 144 | Cas-alpha 10 sgRNA targeting ADE2-CR2 |
| | with positions 163-165 deleted |
| SEQ ID NO: 145 | Cas-alpha 10 sgRNA targeting ADE2-CR2 |
| | with positions 165-167 deleted |
| SEQ ID NO: 146 | Cas-alpha 10 sgRNA targeting ADE2-CR2 |
| | with positions 167-169 deleted |
| SEQ ID NO: 147 | Cas-alpha 10 sgRNA targeting ADE2-CR2 |
| | with positions 169-171 deleted |
| SEQ ID NO: 148 | Cas-alpha 10 sgRNA targeting ADE2-CR2 |
| | with positions 171-173 deleted |
| SEQ ID NO: 149 | Cas-alpha 10 sgRNA targeting ADE2-CR2 |
| | with positions 173-175 deleted |
| SEQ ID NO: 150 | Cas-alpha 10 sgRNA targeting ADE2-CR2 |
| | with positions 175-177 deleted |
| SEQ ID NO: 151 | Cas-alpha 10 sgRNA targeting ADE2-CR2 |
| | with positions 177-179 deleted |
| SEQ ID NO: 152 | Cas-alpha 10 sgRNA targeting ADE2-CR2 |
| | with positions 179-181 deleted |
| SEQ ID NO: 153 | Cas-alpha 10 sgRNA targeting ADE2-CR2 |
| | with positions 181-183 deleted |
| SEQ ID NO: 154 | Cas-alpha 10 sgRNA targeting ADE2-CR2 |
| | with positions 183-185 deleted |
| SEQ ID NO: 155 | Cas-alpha 10 sgRNA targeting ADE2-CR2 |
| | with positions 185-187 deleted |
| SEQ ID NO: 156 | Cas-alpha 10 sgRNA targeting ADE2-CR2 |
| | with positions 187-189 deleted |
| SEQ ID NO: 157 | Wildtype Cas-alpha 10 endonuclease from |
| | Syntrophomonas palmitatica |
| SEQ ID NO: 158 | Betaherpesvirus enhancer |
| SEQ ID NO: 159 | Betaherpesvirus promoter |
| SEQ ID NO: 160 | Human-optimized Cas-alpha 10 gene |
| SEQ ID NO: 161 | Human-optimized sequence encoding SV40 NLS |
| SEQ ID NO: 162 | Human-optimized sequence encoding |
| | ribosome skipping T2A peptide |
| SEQ ID NO: 163 | Human-optimized sequence encoding |
| | green fluorescent protein |
| SEQ ID NO: 164 | Gammaherpesvirus terminator |
| SEQ ID NO: 165 | Human U6 promoter incorporating a G |
| | bp at its 3′ end |
| SEQ ID NO: 166 | DNA sequence encoding Cas-alpha 10 |
| | sgRNA variant MS10 (Cas-alpha |
| | recognition domain) |
| SEQ ID NO: 167 | DNA sequence encoding Cas-alpha 10 |
| | sgRNA variant MS13 (Cas-alpha |
| | recognition domain) |
| SEQ ID NO: 168 | DNA sequence encoding Cas-alpha 10 |
| | sgRNA variant MS13_GAAA (Cas-alpha |
| | recognition domain) |
| SEQ ID NO: 169 | DNA sequence encoding Cas-alpha 10 |
| | sgRNA variant MS13_GAAA_59-61 (Cas- |
| | alpha recognition domain) |
| SEQ ID NO: 170 | DNA sequence encoding Cas-alpha 10 |
| | sgRNA variant TR144 (Cas-alpha |
| | recognition domain) |
| SEQ ID NO: 171 | DNA sequence encoding Cas-alpha 10 |
| | sgRNA variant TR149 (Cas-alpha |
| | recognition domain) |
| SEQ ID NO: 172 | DNA sequence encoding Cas-alpha 10 |
| | sgRNA variant TR150 (Cas-alpha |
| | recognition domain) |
| SEQ ID NO: 173 | DNA sequence encoding Cas-alpha 10 |
| | sgRNA RUNX targeting domain |
| SEQ ID NO: 174 | DNA sequence encoding Cas-alpha 10 |
| | sgRNA WTAP targeting domain |
| SEQ ID NO: 175 | Human U6 terminator sequence (TTTTTT) |
| SEQ ID NO: 176 | DNA sequence encoding Cas-alpha 10 |
| | sgRNA targeting RUNX |
| SEQ ID NO: 177 | DNA sequence encoding Cas-alpha 10 |
| | sgRNA variant MS10 targeting RUNX |
| SEQ ID NO: 178 | DNA sequence encoding Cas-alpha 10 |
| | sgRNA variant MS13 targeting RUNX |
| SEQ ID NO: 179 | DNA sequence encoding Cas-alpha 10 |
| | sgRNA variant MS13_GAAA targeting |
| | RUNX |
| SEQ ID NO: 180 | DNA sequence encoding Cas-alpha 10 |
| | sgRNA variant MS13_GAAA_59-61 |
| | targeting RUNX |
| SEQ ID NO: 181 | DNA sequence encoding Cas-alpha 10 |
| | sgRNA variant TR144 targeting RUNX |
| SEQ ID NO: 182 | DNA sequence encoding Cas-alpha 10 |
| | sgRNA variant TR149 targeting RUNX |
| SEQ ID NO: 183 | DNA sequence encoding Cas-alpha 10 |
| | sgRNA variant TR150 targeting RUNX |
| SEQ ID NO: 184 | DNA sequence encoding Cas-alpha 10 |
| | sgRNA targeting WTAP |
| SEQ ID NO: 185 | DNA sequence encoding Cas-alpha 10 |
| | sgRNA variant MS10 targeting WTAP |
| SEQ ID NO: 186 | DNA sequence encoding Cas-alpha 10 |
| | sgRNA variant MS13 targeting WTAP |
| SEQ ID NO: 187 | DNA sequence encoding Cas-alpha 10 |
| | sgRNA variant MS13_GAAA targeting |
| | WTAP |
| SEQ ID NO: 188 | DNA sequence encoding Cas-alpha 10 |
| | sgRNA variant MS13_GAAA_59-61 |
| | targeting WTAP |
| SEQ ID NO: 189 | DNA sequence encoding Cas-alpha 10 |
| | sgRNA variant TR144 targeting WTAP |
| SEQ ID NO: 190 | DNA sequence encoding Cas-alpha 10 |
| | sgRNA variant TR149 targeting WTAP |
| SEQ ID NO: 191 | DNA sequence encoding Cas-alpha 10 |
| | sgRNA variant TR150 targeting WTAP |
| SEQ ID NO: 192 | Cas-alpha 10 sgRNA targeting RUNX |
| SEQ ID NO: 193 | Cas-alpha 10 sgRNA variant MS10 |
| | targeting RUNX |
| SEQ ID NO: 194 | Cas-alpha 10 sgRNA variant MS13 |
| | targeting RUNX |
| SEQ ID NO: 195 | Cas-alpha 10 sgRNA variant MS13_GAAA |
| | targeting RUNX |
| SEQ ID NO: 196 | Cas-alpha 10 sgRNA variant |
| | MS13_GAAA_59-61 targeting RUNX |
| SEQ ID NO: 197 | Cas-alpha 10 sgRNA variant TR144 |
| | targeting RUNX |
| SEQ ID NO: 198 | Cas-alpha 10 sgRNA variant TR149 |
| | targeting RUNX |
| SEQ ID NO: 199 | Cas-alpha 10 sgRNA variant TR150 |
| | targeting RUNX |
| SEQ ID NO: 200 | Cas-alpha 10 sgRNA targeting WTAP |
| SEQ ID NO: 201 | Cas-alpha 10 sgRNA variant MS10 |
| | targeting WTAP |
| SEQ ID NO: 202 | Cas-alpha 10 sgRNA variant MS13 |
| | targeting WTAP |
| SEQ ID NO: 203 | Cas-alpha 10 sgRNA variant MS13_GAAA |
| | targeting WTAP |
| SEQ ID NO: 204 | Cas-alpha 10 sgRNA variant |
| | MS13_GAAA_59-61 targeting WTAP |
| SEQ ID NO: 205 | Cas-alpha 10 sgRNA variant TR144 |
| | targeting WTAP |
| SEQ ID NO: 206 | Cas-alpha 10 sgRNA variant TR149 |
| | targeting WTAP |
| SEQ ID NO: 207 | Cas-alpha 10 sgRNA variant TR150 |
| | targeting WTAP |
| SEQ ID NO: 208 | Hairpin-like RNA recognition sequence of |
| | the MS2 bacteriophage coat protein |
| SEQ ID NO: 209 | Hairpin-like RNA recognition sequence of |
| | the Cas6 from Pseudomonas aeruginosa |
| SEQ ID NO: 210 | DNA sequence encoding Cas-alpha 10 sgRNA |
| | variant MS2_POS1 (Cas-alpha |
| | recognition domain) |
| SEQ ID NO: 211 | DNA sequence encoding Cas-alpha 10 sgRNA |
| | variant MS2_POS2 (Cas-alpha |
| | recognition domain) |
| SEQ ID NO: 212 | DNA sequence encoding Cas-alpha 10 sgRNA |
| | variant MS2_POS3 (Cas-alpha |
| | recognition domain) |
| SEQ ID NO: 213 | DNA sequence encoding Cas-alpha 10 sgRNA |
| | variant MS2_POS4 (Cas-alpha |
| | recognition domain) |
| SEQ ID NO: 214 | DNA sequence encoding Cas-alpha 10 |
| | sgRNA variant MS2_POS5 (Cas-alpha |
| | recognition domain) |
| SEQ ID NO: 215 | DNA sequence encoding Cas-alpha 10 |
| | sgRNA variant MS2_POS6 (Cas-alpha |
| | recognition domain) |
| SEQ ID NO: 216 | DNA sequence encoding Cas-alpha 10 |
| | sgRNA variant MS2_POS7 (Cas-alpha |
| | recognition domain) |
| SEQ ID NO: 217 | DNA sequence encoding Cas-alpha 10 |
| | sgRNA variant MS2_POS8 (Cas-alpha |
| | recognition domain) |
| SEQ ID NO: 218 | DNA sequence encoding Cas-alpha 10 |
| | sgRNA variant MS2_POS9 (Cas-alpha |
| | recognition domain) |
| SEQ ID NO: 219 | DNA sequence encoding Cas-alpha 10 |
| | sgRNA variant MS2_POS10 (Cas-alpha |
| | recognition domain) |
| SEQ ID NO: 220 | DNA sequence encoding Cas-alpha 10 |
| | sgRNA variant MS2_POS11 (Cas-alpha |
| | recognition domain) |
| SEQ ID NO: 221 | DNA sequence encoding Cas-alpha 10 |
| | sgRNA variant MS2_POS12 (Cas-alpha |
| | recognition domain) |
| SEQ ID NO: 222 | DNA sequence encoding Cas-alpha 10 |
| | sgRNA variant Cas6_POS1 (Cas-alpha |
| | recognition domain) |
| SEQ ID NO: 223 | DNA sequence encoding Cas-alpha 10 |
| | sgRNA variant Cas6_POS2 (Cas-alpha |
| | recognition domain) |
| SEQ ID NO: 224 | DNA sequence encoding Cas-alpha 10 |
| | sgRNA variant Cas6_POS3 (Cas-alpha |
| | recognition domain) |
| SEQ ID NO: 225 | DNA sequence encoding Cas-alpha 10 |
| | sgRNA variant Cas6_POS4 (Cas-alpha |
| | recognition domain) |
| SEQ ID NO: 226 | DNA sequence encoding Cas-alpha 10 |
| | sgRNA variant Cas6_POS5 (Cas-alpha |
| | recognition domain) |
| SEQ ID NO: 227 | DNA sequence encoding Cas-alpha 10 |
| | sgRNA variant Cas6_POS6 (Cas-alpha |
| | recognition domain) |
| SEQ ID NO: 228 | DNA sequence encoding Cas-alpha 10 |
| | sgRNA variant Cas6_POS7 (Cas-alpha |
| | recognition domain) |
| SEQ ID NO: 229 | DNA sequence encoding Cas-alpha 10 |
| | sgRNA variant Cas6_POS8 (Cas-alpha |
| | recognition domain) |
| SEQ ID NO: 230 | DNA sequence encoding Cas-alpha 10 |
| | sgRNA variant Cas6_POS9 (Cas-alpha |
| | recognition domain) |
| SEQ ID NO: 231 | DNA sequence encoding Cas-alpha 10 |
| | sgRNA variant Cas6_POS10 (Cas-alpha |
| | recognition domain) |
| SEQ ID NO: 232 | DNA sequence encoding Cas-alpha 10 |
| | sgRNA variant Cas6_POS11 (Cas-alpha |
| | recognition domain) |
| SEQ ID NO: 233 | DNA sequence encoding Cas-alpha 10 |
| | sgRNA variant Cas6_POS12 (Cas-alpha |
| | recognition domain) |
| SEQ ID NO: 234 | DNA sequence encoding Cas-alpha 10 |
| | sgRNA DNMT targeting domain |
| SEQ ID NO: 235 | Cas-alpha 10 sgRNA DNMT targeting domain |
| SEQ ID NO: 236 | Cas-alpha 10 sgRNA variant MS2_POS1 |
| | targeting DNMT |
| SEQ ID NO: 237 | Cas-alpha 10 sgRNA variant MS2_POS2 |
| | targeting DNMT |
| SEQ ID NO: 238 | Cas-alpha 10 sgRNA variant MS2_POS3 |
| | targeting DNMT |
| SEQ ID NO: 239 | Cas-alpha 10 sgRNA variant MS2_POS4 |
| | targeting DNMT |
| SEQ ID NO: 240 | Cas-alpha 10 sgRNA variant MS2_POS5 |
| | targeting DNMT |
| SEQ ID NO: 241 | Cas-alpha 10 sgRNA variant MS2_POS6 |
| | targeting DNMT |
| SEQ ID NO: 242 | Cas-alpha 10 sgRNA variant MS2_POS7 |
| | targeting DNMT |
| SEQ ID NO: 243 | Cas-alpha 10 sgRNA variant MS2_POS8 |
| | targeting DNMT |
| SEQ ID NO: 244 | Cas-alpha 10 sgRNA variant MS2_POS9 |
| | targeting DNMT |
| SEQ ID NO: 245 | Cas-alpha 10 sgRNA variant MS2_POS10 |
| | targeting DNMT |
| SEQ ID NO: 246 | Cas-alpha 10 sgRNA variant MS2_POS11 |
| | targeting DNMT |
| SEQ ID NO: 247 | Cas-alpha 10 sgRNA variant MS2_POS12 |
| | targeting DNMT |
| SEQ ID NO: 248 | Cas-alpha 10 sgRNA variant Cas6_POS1 |
| | targeting DNMT |
| SEQ ID NO: 249 | Cas-alpha 10 sgRNA variant Cas6_POS2 |
| | targeting DNMT |
| SEQ ID NO: 250 | Cas-alpha 10 sgRNA variant Cas6_POS3 |
| | targeting DNMT |
| SEQ ID NO: 251 | Cas-alpha 10 sgRNA variant Cas6_POS4 |
| | targeting DNMT |
| SEQ ID NO: 252 | Cas-alpha 10 sgRNA variant Cas6_POS5 |
| | targeting DNMT |
| SEQ ID NO: 253 | Cas-alpha 10 sgRNA variant Cas6_POS6 |
| | targeting DNMT |
| SEQ ID NO: 254 | Cas-alpha 10 sgRNA variant Cas6_POS7 |
| | targeting DNMT |
| SEQ ID NO: 255 | Cas-alpha 10 sgRNA variant Cas6_POS8 |
| | targeting DNMT |
| SEQ ID NO: 256 | Cas-alpha 10 sgRNA variant Cas6_POS9 |
| | targeting DNMT |
| SEQ ID NO: 257 | Cas-alpha 10 sgRNA variant Cas6_POS10 |
| | targeting DNMT |
| SEQ ID NO: 258 | Cas-alpha 10 sgRNA variant Cas6_POS11 |
| | targeting DNMT |
| SEQ ID NO: 259 | Cas-alpha 10 sgRNA variant Cas6_POS12 |
| | targeting DNMT |
| SEQ ID NO: 260 | Cas-alpha 10 sgRNA targeting DNMT |
| SEQ ID NO: 261 | Cas-alpha 10 sgRNA variant MS2_POS1 |
| | targeting DNMT |
| SEQ ID NO: 262 | Cas-alpha 10 sgRNA variant MS2_POS2 |
| | targeting DNMT |
| SEQ ID NO: 263 | Cas-alpha 10 sgRNA variant MS2_POS3 |
| | targeting DNMT |
| SEQ ID NO: 264 | Cas-alpha 10 sgRNA variant MS2_POS4 |
| | targeting DNMT |
| SEQ ID NO: 265 | Cas-alpha 10 sgRNA variant MS2_POS5 |
| | targeting DNMT |
| SEQ ID NO: 266 | Cas-alpha 10 sgRNA variant MS2_POS6 |
| | targeting DNMT |
| SEQ ID NO: 267 | Cas-alpha 10 sgRNA variant MS2_POS7 |
| | targeting DNMT |
| SEQ ID NO: 268 | Cas-alpha 10 sgRNA variant MS2_POS8 |
| | targeting DNMT |
| SEQ ID NO: 269 | Cas-alpha 10 sgRNA variant MS2_POS9 |
| | targeting DNMT |
| SEQ ID NO: 270 | Cas-alpha 10 sgRNA variant MS2_POS10 |
| | targeting DNMT |
| SEQ ID NO: 271 | Cas-alpha 10 sgRNA variant MS2_POS11 |
| | targeting DNMT |
| SEQ ID NO: 272 | Cas-alpha 10 sgRNA variant MS2_POS12 |
| | targeting DNMT |
| SEQ ID NO: 273 | Cas-alpha 10 sgRNA variant Cas6_POS1 |
| | targeting DNMT |
| SEQ ID NO: 274 | Cas-alpha 10 sgRNA variant Cas6_POS2 |
| | targeting DNMT |
| SEQ ID NO: 275 | Cas-alpha 10 sgRNA variant Cas6_POS3 |
| | targeting DNMT |
| SEQ ID NO: 276 | Cas-alpha 10 sgRNA variant Cas6_POS4 |
| | targeting DNMT |
| SEQ ID NO: 277 | Cas-alpha 10 sgRNA variant Cas6_POS5 |
| | targeting DNMT |
| SEQ ID NO: 278 | Cas-alpha 10 sgRNA variant Cas6_POS6 |
| | targeting DNMT |
| SEQ ID NO: 279 | Cas-alpha 10 sgRNA variant Cas6_POS7 |
| | targeting DNMT |
| SEQ ID NO: 280 | Cas-alpha 10 sgRNA variant Cas6_POS8 |
| | targeting DNMT |
| SEQ ID NO: 281 | Cas-alpha 10 sgRNA variant Cas6_POS9 |
| | targeting DNMT |
| SEQ ID NO: 282 | Cas-alpha 10 sgRNA variant Cas6_POS10 |
| | targeting DNMT |
| SEQ ID NO: 283 | Cas-alpha 10 sgRNA variant Cas6_POS11 |
| | targeting DNMT |
| SEQ ID NO: 284 | Cas-alpha 10 sgRNA variant Cas6_POS12 |
| | targeting DNMT |
| SEQ ID NO: 285 | Zea mays Ubiquitin promoter |
| SEQ ID NO: 286 | Zea mays Ubiquitin 5' UTR |
| SEQ ID NO: 287 | Zea mays Ubiquitin intron 1 |
| SEQ ID NO: 288 | ST-LS1 Intron 2 |
| SEQ ID NO: 289 | Zea mays optimized gene encoding |
| | Cas-alpha 10 SEQ ID NO: 14 including ST- |
| | LS1 Intron 2 |
| SEQ ID NO: 290 | Zea mays optimized sequence encoding SV40 NLS |
| SEQ ID NO: 291 | Zea mays Ubiquitin terminator |
| SEQ ID NO: 292 | Zea mays U6 promoter incorporating a |
| | G bp at its 3′ end |
| SEQ ID NO: 293 | DNA sequence encoding Cas-alpha 10 |
| | sgRNA MS26 targeting domain |
| SEQ ID NO: 294 | DNA sequence encoding Cas-alpha 10 |
| | sgRNA CR36 targeting domain |
| SEQ ID NO: 295 | DNA sequence encoding Cas-alpha 10 |
| | sgRNA D8 targeting domain |
| SEQ ID NO: 296 | Zea mays U6 terminator (TTTTTTTT) |
| SEQ ID NO: 297 | DNA sequence encoding Cas-alpha 10 |
| | sgRNA targeting MS26 |
| SEQ ID NO: 298 | DNA sequence encoding Cas-alpha 10 |
| | sgRNA targeting CR36 |
| SEQ ID NO: 299 | DNA sequence encoding Cas-alpha 10 |
| | sgRNA targeting D8 |
| SEQ ID NO: 300 | DNA sequence encoding Cas-alpha 10 |
| | sgRNA variant MS13_GAAA targeting |
| | MS26 |
| SEQ ID NO: 301 | DNA sequence encoding Cas-alpha 10 |
| | sgRNA variant MS13_GAAA targeting |
| | CR36 |
| SEQ ID NO: 302 | DNA sequence encoding Cas-alpha 10 |
| | sgRNA variant MS13_GAAA targeting D8 |
| SEQ ID NO: 303 | MS26 targeting domain |
| SEQ ID NO: 304 | CR36 targeting domain |
| SEQ ID NO: 305 | D8 targeting domain |
| SEQ ID NO: 306 | RUNX targeting domain |
| SEQ ID NO: 307 | WTAP targeting domain |
| SEQ ID NO: 308 | DNMT targeting domain |
| SEQ ID NO: 309 | Cas-alpha 10 G40A variant |
| SEQ ID NO: 310 | Cas-alpha 10 K87A variant |
| SEQ ID NO: 311 | Cas-alpha 10 K190T variant |
| SEQ ID NO: 312 | Cas-alpha 10 H217T variant |
| SEQ ID NO: 313 | Cas-alpha 10 S298K variant |
| SEQ ID NO: 314 | Cas-alpha 10 F306H variant |
| SEQ ID NO: 315 | Cas-alpha 10 R335T variant |
| SEQ ID NO: 316 | Cas-alpha 10 N405I variant |
| SEQ ID NO: 317 | Cas-alpha 10 G81E + S298K variant |
| SEQ ID NO: 318 | Cas-alpha 10 G81E + S298K + F306H variant |
| SEQ ID NO: 319 | Cas-alpha 10 G81E + S298K + |
| | F306H + N405I variant |
| SEQ ID NO: 320 | Cas-alpha 10 G81E + S298K + N405I variant |
| SEQ ID NO: 321 | Cas-alpha 10 G81E + N405I variant |
| SEQ ID NO: 322 | Cas-alpha 10 G81E + F306H + N405I variant |
| SEQ ID NO: 323 | Cas-alpha 10 G81E + F306H variant |
| SEQ ID NO: 324 | Cas-alpha 10 sgRNA MS10 (Cas-alpha |
| | recognition domain) |
| SEQ ID NO: 325 | Cas-alpha 10 sgRNA MS13 (Cas-alpha |
| | recognition domain) |
| SEQ ID NO: 326 | Cas-alpha 10 sgRNA design 5 (Cas-alpha |
| | recognition domain) |
| SEQ ID NO: 327 | Cas-alpha 10 sgRNA MS13_GAAA |
| | (Cas-alpha recognition domain) |
| SEQ ID NO: 328 | DNA sequence encoding Cas-alpha 10 |
| | sgRNA variant MS10_59-61 targeting |
| | RUNX |
| SEQ ID NO: 329 | DNA sequence encoding Cas-alpha 10 |
| | sgRNA variant MS13_59-61 targeting |
| | RUNX |
| SEQ ID NO: 330 | DNA sequence encoding Cas-alpha 10 |
| | sgRNA variant design 5 targeting RUNX |
| SEQ ID NO: 331 | DNA sequence encoding Cas-alpha 10 |
| | sgRNA variant design 5_59-61 targeting |
| | RUNX |
| SEQ ID NO: 332 | DNA sequence encoding Cas-alpha 10 |
| | sgRNA variant design 5_61-63 targeting |
| | RUNX |
| SEQ ID NO: 333 | DNA sequence encoding Cas-alpha 10 |
| | sgRNA variant design 5_157-159 |
| | targeting RUNX |
| SEQ ID NO: 334 | Cas-alpha 10 sgRNA variant MS10_59-61 |
| | targeting RUNX |
| SEQ ID NO: 335 | Cas-alpha 10 sgRNA variant MS13_59-61 |
| | targeting RUNX |
| SEQ ID NO: 336 | Cas-alpha 10 sgRNA variant design 5 |
| | targeting RUNX |
| SEQ ID NO: 337 | Cas-alpha 10 sgRNA variant design |
| | 5_59-61 targeting RUNX |
| SEQ ID NO: 338 | Cas-alpha 10 sgRNA variant design 5_61-63 |
| | targeting RUNX |
| SEQ ID NO: 339 | Cas-alpha 10 sgRNA variant design |
| | 5_157-159 targeting RUNX |
| SEQ ID NO: 340 | DNA sequence encoding Cas-alpha 10 |
| | sgRNA variant MS10_59-61 targeting |
| | WTAP |
| SEQ ID NO: 341 | DNA sequence encoding Cas-alpha 10 |
| | sgRNA variant MS13_59-61 targeting |
| | WTAP |
| SEQ ID NO: 342 | DNA sequence encoding Cas-alpha 10 |
| | sgRNA variant design 5 targeting WTAP |
| SEQ ID NO: 343 | DNA sequence encoding Cas-alpha 10 |
| | sgRNA variant design 5_59-61 targeting |
| | WTAP |
| SEQ ID NO: 344 | DNA sequence encoding Cas-alpha 10 |
| | sgRNA variant design 5_61-63 targeting |
| | WTAP |
| SEQ ID NO: 345 | DNA sequence encoding Cas-alpha 10 |
| | sgRNA variant design 5_157-159 |
| | targeting WTAP |
| SEQ ID NO: 346 | Cas-alpha 10 sgRNA variant MS10_59-61 |
| | targeting WTAP |
| SEQ ID NO: 347 | Cas-alpha 10 sgRNA variant MS13_59-61 |
| | targeting WTAP |
| SEQ ID NO: 348 | Cas-alpha 10 sgRNA variant design 5 |
| | targeting WTAP |
| SEQ ID NO: 349 | Cas-alpha 10 sgRNA variant design |
| | 5_59-61 targeting WTAP |
| SEQ ID NO: 350 | Cas-alpha 10 sgRNA variant design |
| | 5_61-63 targeting WTAP |
| SEQ ID NO: 351 | Cas-alpha 10 sgRNA variant design |
| | 5_157-159 targeting WTAP |
| SEQ ID NO: 352 | Cas-alpha 10 sgRNA targeting MS26 |
| SEQ ID NO: 353 | Cas-alpha 10 sgRNA targeting D8 |
| SEQ ID NO: 354 | Cas-alpha 10 sgRNA variant MS13_GAAA |
| | targeting MS26 |
| SEQ ID NO: 355 | Cas-alpha 10 sgRNA variant MS13_GAAA |
| | targeting D8 |
| SEQ ID NO: 356 | DNA sequence encoding Cas-alpha 10 |
| | sgRNA variant MS10_59-61 (Cas-alpha |
| | recognition domain) |
| SEQ ID NO: 357 | DNA sequence encoding Cas-alpha 10 |
| | sgRNA variant MS13_59-61 (Cas-alpha |
| | recognition domain) |
| SEQ ID NO: 358 | DNA sequence encoding Cas-alpha 10 |
| | sgRNA variant design 5 (Cas-alpha |
| | recognition domain) |
| SEQ ID NO: 359 | DNA sequence encoding Cas-alpha 10 |
| | sgRNA variant design 5_59-61 (Cas- |
| | alpha recognition domain) |
| SEQ ID NO: 360 | DNA sequence encoding Cas-alpha 10 |
| | sgRNA variant design 5_61-63 (Cas- |
| | alpha recognition domain) |
| SEQ ID NO: 361 | DNA sequence encoding Cas-alpha 10 |
| | sgRNA variant design 5_157-159 (Cas- |
| | alpha recognition domain) |
| SEQ ID NO: 362 | Cas-alpha 10 T34E + M45S + G81E + |
| | G470D variant |

### DETAILED DESCRIPTION

The temperature optimum of many native Cas endonuclease is above the typical biological temperatures of some organisms, including plants and yeast. Because of this, a wild-type Cas endonuclease (e.g., SEQ TD NO:48) would require a heat shock of approximately 45 degrees Celsius for optimal activity. For some applications, it may be beneficial to modify this property. Herein are presented methods and compositions for novel engineered CRISPR effectors, systems, and elements comprising such effectors, including, but not limiting to, novel guide polynucleotide/endonuclease complexes, guide polynucleotides, guide RNA elements, Cas proteins, and endonucleases, as well as proteins comprising an endonuclease functionality (domain). Compositions and methods are also provided for direct delivery of endonucleases, cleavage ready complexes, guide RNAs, and guide RNA/Cas endonuclease complexes. The present disclosure further includes compositions and methods for genome modification of a target sequence in the genome of a cell, for gene editing, and for inserting a polynucleotide of interest into the genome of a cell. The variants identified should improve genome editing outcomes in a variety of cell types including human and aid in the wide-spread adoption of this miniature RNA-guided Cas nuclease.

Terms used in the claims and specification are defined as set forth below unless otherwise specified. It must be noted that, as used in the specification and the appended claims, the singular forms “a,” “an” and “the” include plural referents unless the context clearly dictates otherwise.

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA and/or RNA-DNA that is single- or double-stranded, optionally comprising synthetic, non-natural, or altered nucleotide bases. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

The term “genome” as it applies to a prokaryotic and eukaryotic cell or organism cells encompasses not only chromosomal DNA found within the nucleus, but organelle DNA found within subcellular components (e.g., mitochondria, or plastid) of the cell.

“Open reading frame” is abbreviated ORF.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to and including 100% sequence identity (i.e., fully complementary) with each other.

The term “stringent conditions” or “stringent hybridization conditions” includes reference to conditions under which a probe will selectively hybridize to its target sequence in an in vitro hybridization assay. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length. Typically, stringent conditions will be those in which the salt concentration is less than about 1.5 M Na ion, typically about 0.01 to 1.0 M Na ion concentration (or other salt(s)) at pH 7.0 to 8.3, and at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

As used herein, “homologous recombination” (HR) includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombination events: the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al. (1982) Cell 31:25-33; Shen and Huang, (1986) Genetics 112:441-57; Watt et al. (1985) Proc. Natl. Acad. Sci. USA 82:4768-72, Sugawara and Haber, (1992) Mol Cell Biol 12:563-75, Rubnitz and Subramani, (1984) Mol Cell Biol 4:2253-8; Ayares et al. (1986) Proc. Natl. Acad. Sci. USA 83:5199-203; Liskay et al. (1987) Genetics 115:161-7.

“Sequence identity” or “identity” in the context of nucleic acid or polypeptide sequences refers to the nucleic acid bases or amino acid residues in two sequences that are the same when aligned for maximum correspondence over a specified comparison window.

The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90%, or 95%, or any percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, WI). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

The “Clustal V method of alignment” corresponds to the alignment method labeled Clustal V (described by Higgins and Sharp, (1989) CABIOS 5:151-153; Higgins et al. (1992) Comput Appl Biosci 8:189-191) and found in the MegAlign™ program of the LASERGENE bioinformatics computing suite (DNASTAR Inc., Madison, WI). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” Table in the same program. The “Clustal W method of alignment” corresponds to the alignment method labeled Clustal W (described by Higgins and Sharp, (1989) CABIOS 5:151-153; Higgins et al. (1992) Comput Appl Biosci 8:189-191) and found in the MegAlign™ v6.1 program of the LASERGENE bioinformatics computing suite (DNASTAR Inc., Madison, WI). Default parameters for multiple alignment (GAP PENALTY=10, GAP LENGTH PENALTY=0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” Table in the same program. Unless otherwise stated, sequence identity/similarity values provided herein refer to the value obtained using GAP Version 10 (GCG, Accelrys, San Diego, CA) using the following parameters:% identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix (Henikoff and Henikoff, (1989)Proc. Natl. Acad. Sci. USA 89:10915). GAP uses the algorithm of Needleman and Wunsch, (1970) J Mol Biol 48:443-53, to find an alignment of two complete sequences that maximizes the number of matches and minimizes the number of gaps. GAP considers all possible alignments and gap positions and creates the alignment with the largest number of matched bases and the fewest gaps, using a gap creation penalty and a gap extension penalty in units of matched bases. “BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence. It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any percentage from 50% to 100%. Indeed, any amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

Polynucleotide and polypeptide sequences, variants thereof, and the structural relationships of these sequences can be described by the terms “homology”, “homologous”, “substantially identical”, “substantially similar” and “corresponding substantially” which are used interchangeably herein. These refer to polypeptide or nucleic acid sequences wherein changes in one or more amino acids or nucleotide bases do not affect the function of the molecule, such as the ability to mediate gene expression or to produce a certain phenotype. These terms also refer to modification(s) of nucleic acid sequences that do not substantially alter the functional properties of the resulting nucleic acid relative to the initial, unmodified nucleic acid. These modifications include deletion, substitution, and/or insertion of one or more nucleotides in the nucleic acid fragment. Substantially similar nucleic acid sequences encompassed may be defined by their ability to hybridize (under moderately stringent conditions, e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions.

A “centimorgan” (cM) or “map unit” is the distance between two polynucleotide sequences, linked genes, markers, target sites, loci, or any pair thereof, wherein 1% of the products of meiosis are recombinant. Thus, a centimorgan is equivalent to a distance equal to a 1% average recombination frequency between the two linked genes, markers, target sites, loci, or any pair thereof.

An “isolated” or “purified” nucleic acid molecule, polynucleotide, polypeptide, or protein, or biologically active portion thereof, is substantially or essentially free from components that normally accompany or interact with the polynucleotide or protein as found in its naturally occurring environment. Thus, an isolated or purified polynucleotide or polypeptide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. Isolated polynucleotides may be purified from a cell in which they naturally occur. Conventional nucleic acid purification methods known to skilled artisans may be used to obtain isolated polynucleotides. The term also embraces recombinant polynucleotides and chemically synthesized polynucleotides.

The term “fragment” refers to a contiguous set of nucleotides or amino acids. In one embodiment, a fragment is 2, 3, 4, 5, 6, 7 8, 9, 10, 11, 12, 13, 14, 15, 16, 17, 18, 19, 20, or greater than 20 contiguous nucleotides. In one embodiment, a fragment is 2, 3, 4, 5, 6, 7 8, 9, 10, 11, 12, 13, 14, 15, 16, 17, 18, 19, 20, or greater than 20 contiguous amino acids. A fragment may or may not exhibit the function of a sequence sharing some percent identity over the length of said fragment.

The terms “fragment that is functionally equivalent” and “functionally equivalent fragment” are used interchangeably herein. These terms refer to a portion or subsequence of an isolated nucleic acid fragment or polypeptide that displays the same activity or function as the longer sequence from which it derives. In one example, the fragment retains the ability to alter gene expression or produce a certain phenotype whether or not the fragment encodes an active protein. For example, the fragment can be used in the design of genes to produce the desired phenotype in a modified plant. Genes can be designed for use in suppression by linking a nucleic acid fragment, whether or not it encodes an active enzyme, in the sense or antisense orientation relative to a plant promoter sequence.

“Gene” includes a nucleic acid fragment that expresses a functional molecule such as, but not limited to, a specific protein, including regulatory sequences preceding (5′ non-coding sequences) and following (3′ non-coding sequences) the coding sequence. “Native gene” refers to a gene as found in its natural endogenous location with its own regulatory sequences.

By the term “endogenous” it is meant a sequence or other molecule that naturally occurs in a cell or organism. In one aspect, an endogenous polynucleotide is normally found in the genome of a cell; that is, not heterologous.

An “allele” is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus.

“Coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas endonuclease system as disclosed herein. A mutated plant is a plant comprising a mutated gene.

As used herein, a “targeted mutation” is a mutation in a gene (referred to as the target gene), including a native gene, that was made by altering a target sequence within the target gene using any method known to one skilled in the art, including a method involving a guided Cas endonuclease system as disclosed herein.

The terms “knock-out”, “gene knock-out” and “genetic knock-out” are used interchangeably herein. A knock-out represents a DNA sequence of a cell that has been rendered partially or completely inoperative by targeting with a Cas protein; for example, a DNA sequence prior to knock-out could have encoded an amino acid sequence, or could have had a regulatory function (e.g., promoter).

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (for example by homologous recombination (HR), wherein a suitable donor DNA polynucleotide is also used). examples of knock-ins are a specific insertion of a heterologous amino acid coding sequence in a coding region of a gene, or a specific insertion of a transcriptional regulatory element in a genetic locus.

By “domain” it is meant a contiguous stretch of nucleotides (that can be RNA, DNA, and/or RNA-DNA-combination sequence) or amino acids.

The term “conserved domain” or “motif” means a set of polynucleotides or amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

A “codon-modified gene” or “codon-preferred gene” or “codon-optimized gene” is a gene having its frequency of codon usage designed to mimic the frequency of preferred codon usage of the host cell.

An “optimized” polynucleotide is a sequence that has been optimized for improved expression in a particular heterologous host cell.

A “plant-optimized nucleotide sequence” is a nucleotide sequence that has been optimized for expression in plants, particularly for increased expression in plants. A plant-optimized nucleotide sequence includes a codon-optimized gene. A plant-optimized nucleotide sequence can be synthesized by modifying a nucleotide sequence encoding a protein such as, for example, a Cas endonuclease as disclosed herein, using one or more plant-preferred codons for improved expression. See, for example, Campbell and Gowri (1990) Plant Physiol. 92:1-11 for a discussion of host-preferred codon usage.

A “promoter” is a region of DNA involved in recognition and binding of RNA polymerase and other proteins to initiate transcription. The promoter sequence consists of proximal and more distal upstream elements, the latter elements often referred to as enhancers. An “enhancer” is a DNA sequence that can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

Promoters that cause a gene to be expressed in most cell types at most times are commonly referred to as “constitutive promoters”. The term “inducible promoter” refers to a promoter that selectively express a coding sequence or functional RNA in response to the presence of an endogenous or exogenous stimulus, for example by chemical compounds (chemical inducers) or in response to environmental, hormonal, chemical, and/or developmental signals. Inducible or regulated promoters include, for example, promoters induced or regulated by light, heat, stress, flooding or drought, salt stress, osmotic stress, phytohormones, wounding, or chemicals such as ethanol, abscisic acid (ABA), jasmonate, salicylic acid, or safeners.

“Translation leader sequence” refers to a polynucleotide sequence located between the promoter sequence of a gene and the coding sequence. The translation leader sequence is present in the mRNA upstream of the translation start sequence. The translation leader sequence may affect processing of the primary transcript to mRNA, mRNA stability or translation efficiency. Examples of translation leader sequences have been described (e.g., Turner and Foster, (1995) Mol Biotechnol 3:225-236).

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. (1989) Plant Cell 1:671-680.

“RNA transcript” refers to the product resulting from RNA polymerase-catalyzed transcription of a DNA sequence. When the RNA transcript is a perfect complimentary copy of the DNA sequence, it is referred to as the primary transcript or pre-mRNA. An RNA transcript is referred to as the mature RNA or mRNA when it is an RNA sequence derived from post-transcriptional processing of the primary transcript pre-mRNA. “Messenger RNA” or “mRNA” refers to the RNA that is without introns and that can be translated into protein by the cell. “cDNA” refers to a DNA that is complementary to, and synthesized from, an mRNA template using the enzyme reverse transcriptase. The cDNA can be single-stranded or converted into double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “genome” refers to the entire complement of genetic material (genes and non-coding sequences) that is present in each cell of an organism, or virus or organelle; and/or a complete set of chromosomes inherited as a (haploid) unit from one parent.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., the coding sequence is under the transcriptional control of the promoter). Coding sequences can be operably linked to regulatory sequences in a sense or antisense orientation. In another example, the complementary RNA regions can be operably linked, either directly or indirectly, 5′ to the target mRNA, or 3′ to the target mRNA, or within the target mRNA, or a first complementary region is 5′ and its complement is 3′ to the target mRNA.

Generally, “host” refers to an organism or cell into which a heterologous component (polynucleotide, polypeptide, other molecule, cell) has been introduced. As used herein, a “host cell” refers to an in vivo or in vitro eukaryotic cell, prokaryotic cell (e.g., bacterial or archaeal cell), or cell from a multicellular organism (e.g., a cell line) cultured as a unicellular entity, into which a heterologous polynucleotide or polypeptide has been introduced. In some embodiments, the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, an insect cell, a mammalian cell, a pig cell, a cow cell, a goat cell, a sheep cell, a rodent cell, a rat cell, a mouse cell, a non-human primate cell, and a human cell. In some cases, the cell is in vitro. In some cases, the cell is in vivo.

The term “recombinant” refers to an artificial combination of two otherwise separated segments of sequence, e.g., by chemical synthesis, or manipulation of isolated segments of nucleic acids by genetic engineering techniques.

The terms “plasmid”, “vector” and “cassette” refer to a linear or circular extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. “Transformation cassette” refers to a specific vector comprising a gene and having elements in addition to the gene that facilitates transformation of a particular host cell. “Expression cassette” refers to a specific vector comprising a gene and having elements in addition to the gene that allow for expression of that gene in a host.

The terms “recombinant DNA molecule”, “recombinant DNA construct”, “expression construct”, “construct”, and “recombinant construct” are used interchangeably herein. A recombinant DNA construct comprises an artificial combination of nucleic acid sequences, e.g., regulatory and coding sequences that are not all found together in nature. For example, a recombinant DNA construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to introduce the vector into the host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al. (1985) EMBO J 4:2411-2418; De Almeida et al. (1989) Mol Gen Genetics 218:78-86), and thus that multiple events are typically screened in order to obtain lines displaying the desired expression level and pattern. Such screening may be accomplished standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

The term “heterologous” refers to the difference between the original environment, location, or composition of a particular polynucleotide or polypeptide sequence and its current environment, location, or composition. Non-limiting examples include differences in taxonomic derivation (e.g., a polynucleotide sequence obtained from Zea mays would be heterologous if inserted into the genome of an Oryza sativa plant, or of a different variety or cultivar of Zea mays; or a polynucleotide obtained from a bacterium was introduced into a cell of a plant), or sequence (e.g., a polynucleotide sequence obtained from Zea mays, isolated, modified, and re-introduced into a maize plant). As used herein, “heterologous” in reference to a sequence can refer to a sequence that originates from a different species, variety, foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, one or more regulatory region(s) and/or a polynucleotide provided herein may be entirely synthetic. In another example, a target polynucleotide for cleavage by a Cas endonuclease may be of a different organism than that of the Cas endonuclease. In another example, a Cas endonuclease and guide RNA may be introduced to a target polynucleotide with an additional polynucleotide that acts as a template or donor for insertion into the target polynucleotide, wherein the additional polynucleotide is heterologous to the target polynucleotide and/or the Cas endonuclease.

The term “expression”, as used herein, refers to the production of a functional end-product (e.g., an mRNA, guide RNA, or a protein) in either precursor or mature form.

A “mature” protein refers to a post-translationally processed polypeptide (i.e., one from which any pre- or propeptides present in the primary translation product have been removed).

“Precursor” protein refers to the primary product of translation of mRNA (i.e., with pre- and propeptides still present). Pre- and propeptides may be but are not limited to intracellular localization signals.

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327:167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

As used herein, an “effector” or “effector protein” is a protein that encompasses an activity including recognizing, binding to, and/or cleaving or nicking a polynucleotide target. An effector, or effector protein, may also be an endonuclease. The “effector complex” of a CRISPR system includes Cas proteins involved in crRNA and target recognition and binding. Some of the component Cas proteins may additionally comprise domains involved in target polynucleotide cleavage.

The term “Cas protein” refers to a polypeptide encoded by a Cas (CRISPR-associated) gene. A Cas protein includes proteins encoded by a gene in a cas locus, and include adaptation molecules as well as interference molecules. An interference molecule of a bacterial adaptive immunity complex includes endonucleases. A Cas endonuclease described herein comprises one or more nuclease domains. A Cas endonuclease includes but is not limited to: the novel Cas endonuclease protein disclosed herein, a Cas9 protein, a Cpf1 (Casl2) protein, a C2c1 protein, a C2c2 protein, a C2c3 protein, Cas3, Cas3-HD, Cas 5, Cas7, Cas8, Cas10, or combinations or complexes of these. A Cas protein may be a “Cas endonuclease” or “Cas effector protein”, that when in complex with a suitable polynucleotide component, is capable of recognizing, binding to, and optionally nicking or cleaving all or part of a specific polynucleotide target sequence. The Cas endonucleases of the disclosure include those having one or more RuvC nuclease domains. A Cas protein is further defined as a functional fragment or functional variant of a native Cas protein, or a protein that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native Cas protein, and retains at least partial activity of the native sequence.

A “functional fragment”, “fragment that is functionally equivalent” and “functionally equivalent fragment” of a Cas endonuclease are used interchangeably herein, and refer to a portion or subsequence of the Cas endonuclease of the present disclosure in which the ability to recognize, bind to, and optionally unwind, nick or cleave (introduce a single or double-strand break in) the target site is retained. The portion or subsequence of the Cas endonuclease can comprise a complete or partial (functional) peptide of any one of its domains such as for example, but not limiting to a complete of functional part of a Cas3 HD domain, a complete of functional part of a Cas3 Helicase domain, complete of functional part of a protein (such as but not limiting to a Cas5, Cas5d, Cas7 and Cas8b1).

The terms “functional variant”, “variant that is functionally equivalent” and “functionally equivalent variant” of a Cas endonuclease or Cas effector protein, including Cas endonuclease described herein, are used interchangeably herein, and refer to a variant of the Cas effector protein disclosed herein in which the ability to recognize, bind to, and optionally unwind, nick or cleave all or part of a target sequence is retained.

A Cas endonuclease may also include a multifunctional Cas endonuclease. The term “multifunctional Cas endonuclease” and “multifunctional Cas endonuclease polypeptide” are used interchangeably herein and includes reference to a single polypeptide that has Cas endonuclease functionality (comprising at least one protein domain that can act as a Cas endonuclease) and at least one other functionality, such as but not limited to, the functionality to form a complex (comprises at least a second protein domain that can form a complex with other proteins). In one aspect, the multifunctional Cas endonuclease comprises at least one additional protein domain relative (either internally, upstream (5′), downstream (3′), or both internally 5′ and 3′, or any combination thereof) to those domains typical of a Cas endonuclease.

The terms “cascade” and “cascade complex” are used interchangeably herein and include reference to a multi-subunit protein complex that can assemble with a polynucleotide forming a polynucleotide-protein complex (PNP). Cascade is a PNP that relies on the polynucleotide for complex assembly and stability, and for the identification of target nucleic acid sequences. Cascade functions as a surveillance complex that finds and optionally binds target nucleic acids that are complementary to a variable targeting domain of the guide polynucleotide.

The terms “cleavage-ready Cascade”, “crCascade”, “cleavage-ready Cascade complex”, “crCascade complex”, “cleavage-ready Cascade system”, “CRC” and “crCascade system”, are used interchangeably herein and include reference to a multi-subunit protein complex that can assemble with a polynucleotide forming a polynucleotide-protein complex (PNP), wherein one of the cascade proteins is a Cas endonuclease capable of recognizing, binding to, and optionally unwinding, nicking, or cleaving all or part of a target sequence.

The terms “5′-cap” and “7-methylguanylate (m7G) cap” are used interchangeably herein. A 7-methylguanylate residue is located on the 5′ terminus of messenger RNA (mRNA) in eukaryotes. RNA polymerase II (Pol II) transcribes mRNA in eukaryotes. Messenger RNA capping occurs generally as follows: the most terminal 5′ phosphate group of the mRNA transcript is removed by RNA terminal phosphatase, leaving two terminal phosphates. A guanosine monophosphate (GMP) is added to the terminal phosphate of the transcript by a guanylyl transferase, leaving a 5′-5′ triphosphate-linked guanine at the transcript terminus. Finally, the 7-nitrogen of this terminal guanine is methylated by a methyl transferase.

The terminology “not having a 5′-cap” herein is used to refer to RNA having, for example, a 5′-hydroxyl group instead of a 5′-cap. Such RNA can be referred to as “uncapped RNA”, for example. Uncapped RNA can better accumulate in the nucleus following transcription, since 5′-capped RNA is subject to nuclear export. One or more RNA components herein are uncapped.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be an RNA sequence, a DNA sequence, or a combination thereof (an RNA-DNA combination sequence).

The terms “functional fragment”, “fragment that is functionally equivalent” and “functionally equivalent fragment” of a guide RNA, crRNA or tracrRNA are used interchangeably herein, and refer to a portion or subsequence of the guide RNA, crRNA or tracrRNA, respectively, of the present disclosure in which the ability to function as a guide RNA, crRNA or tracrRNA, respectively, is retained.

The terms “functional variant”, “variant that is functionally equivalent” and “functionally equivalent variant” of a guide RNA, crRNA or tracrRNA (respectively) are used interchangeably herein, and refer to a variant of the guide RNA, crRNA or tracrRNA, respectively, of the present disclosure in which the ability to function as a guide RNA, crRNA or tracrRNA, respectively, is retained.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, optionally bind to, and optionally nick or cleave (introduce a single or double-strand break) the DNA target site.

The term “variable targeting domain” or “VT domain” is used interchangeably herein and includes a nucleotide sequence that can hybridize (is complementary) to one strand (nucleotide sequence) of a double strand DNA target site. The percent complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable targeting domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, an RNA sequence, a modified DNA sequence, a modified RNA sequence, or any combination thereof.

The term “Cas endonuclease recognition domain” or “CER domain” (of a guide polynucleotide) is used interchangeably herein and includes a nucleotide sequence that interacts with a Cas endonuclease polypeptide. A CER domain comprises a (trans-acting) tracrNucleotide mate sequence followed by a tracrNucleotide sequence. The CER domain can be composed of a DNA sequence, an RNA sequence, a modified DNA sequence, a modified RNA sequence (see for example US20150059010A1, published 26 Feb. 2015), or any combination thereof.

As used herein, the terms “guide polynucleotide/Cas endonuclease complex”, “guide polynucleotide/Cas endonuclease system”, “guide polynucleotide/Cas complex”, “guide polynucleotide/Cas system” and “guided Cas system” “Polynucleotide-guided endonuclease” “PGEN” are used interchangeably herein and refer to at least one guide polynucleotide and at least one Cas endonuclease, that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double-strand break) the DNA target site. A guide polynucleotide/Cas endonuclease complex herein can comprise Cas protein(s) and suitable polynucleotide component(s) of any of the known CRISPR systems (Horvath and Barrangou, 2010, Science 327:167-170; Makarova et al. 2015, Nature Reviews Microbiology Vol. 13:1-15; Zetsche et al. 2015, Cell 163, 1-13; Shmakov et al. 2015, Molecular Cell 60, 1-13).

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double-strand break) the DNA target site.

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, (iv) a chemical alteration of at least one nucleotide, or (v) any combination of (i)-(iv).

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, (iv) a chemical alteration of at least one nucleotide, or (v) any combination of (i)-(iv).

Methods for “modifying a target site” and “altering a target site” are used interchangeably herein and refer to methods for producing an altered target site.

As used herein, “donor DNA” is a DNA construct that comprises a polynucleotide of interest to be inserted into the target site of a Cas endonuclease.

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The term “plant-optimized Cas endonuclease” herein refers to a Cas protein, including a multifunctional Cas protein, encoded by a nucleotide sequence that has been optimized for expression in a plant cell or plant.

A “plant-optimized nucleotide sequence encoding a Cas endonuclease”, “plant-optimized construct encoding a Cas endonuclease” and a “plant-optimized polynucleotide encoding a Cas endonuclease” are used interchangeably herein and refer to a nucleotide sequence encoding a Cas protein, or a variant or functional fragment thereof, that has been optimized for expression in a plant cell or plant. A plant comprising a plant-optimized Cas endonuclease includes a plant comprising the nucleotide sequence encoding for the Cas sequence and/or a plant comprising the Cas endonuclease protein. In one aspect, the plant-optimized Cas endonuclease nucleotide sequence is a maize-optimized, rice-optimized, wheat-optimized, soybean-optimized, cotton-optimized, or canola-optimized Cas endonuclease.

The term “plant” generically includes whole plants, plant organs, plant tissues, seeds, plant cells, seeds and progeny of the same. The plant is a monocot or dicot. Plant cells include, without limitation, cells from seeds, suspension cultures, embryos, meristematic regions, callus tissue, leaves, roots, shoots, gametophytes, sporophytes, pollen and microspores. A “plant element” is intended to reference either a whole plant or a plant component, which may comprise differentiated and/or undifferentiated tissues, for example but not limited to plant tissues, parts, and cell types. In one embodiment, a plant element is one of the following: whole plant, seedling, meristematic tissue, ground tissue, vascular tissue, dermal tissue, seed, leaf, root, shoot, stem, flower, fruit, stolon, bulb, tuber, corm, keiki, shoot, bud, tumor tissue, and various forms of cells and culture (e.g., single cells, protoplasts, embryos, callus tissue). It should be noted that a protoplast is not technically an “intact” plant cell (as naturally found with all components), as protoplasts lack a cell wall. The term “plant organ” refers to plant tissue or a group of tissues that constitute a morphologically and functionally distinct part of a plant. As used herein, a “plant element” is synonymous to a “portion” of a plant, and refers to any part of the plant, and can include distinct tissues and/or organs, and may be used interchangeably with the term “tissue” throughout. Similarly, a “plant reproductive element” is intended to generically reference any part of a plant that is able to initiate other plants via either sexual or asexual reproduction of that plant, for example but not limited to seed, seedling, root, shoot, cutting, scion, graft, stolon, bulb, tuber, corm, keiki, or bud. The plant element may be in plant or in a plant organ, tissue culture, or cell culture.

“Progeny” comprises any subsequent generation of a plant.

As used herein, the term “plant part” refers to plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like, as well as the parts themselves. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The term “monocotyledonous” or “monocot” refers to the subclass of angiosperm plants also known as “monocotyledoneae”, whose seeds typically comprise only one embryonic leaf, or cotyledon. The term includes references to whole plants, plant elements, plant organs (e.g., leaves, stems, roots, etc.), seeds, plant cells, and progeny of the same.

The term “dicotyledonous” or “dicot” refers to the subclass of angiosperm plants also knows as “dicotyledoneae”, whose seeds typically comprise two embryonic leaves, or cotyledons. The term includes references to whole plants, plant elements, plant organs (e.g., leaves, stems, roots, etc.), seeds, plant cells, and progeny of the same.

As used herein, a “male sterile plant” is a plant that does not produce male gametes that are viable or otherwise capable of fertilization. As used herein, a “female sterile plant” is a plant that does not produce female gametes that are viable or otherwise capable of fertilization. It is recognized that male-sterile and female-sterile plants can be female-fertile and male-fertile, respectively. It is further recognized that a male fertile (but female sterile) plant can produce viable progeny when crossed with a female fertile plant and that a female fertile (but male sterile) plant can produce viable progeny when crossed with a male fertile plant.

The term “non-conventional yeast” herein refers to any yeast that is not a Saccharomyces (e.g., S. cerevisiae) or Schizosaccharomyces yeast species. (see “Non-Conventional Yeasts in Genetics, Biochemistry and Biotechnology: Practical Protocols”, K. Wolf, K. D. Breunig, G. Barth, Eds., Springer-Verlag, Berlin, Germany, 2003).

The term “crossed” or “cross” or “crossing” in the context of this disclosure means the fusion of gametes via pollination to produce progeny (i.e., cells, seeds, or plants). The term encompasses both sexual crosses (the pollination of one plant by another) and selfing (self-pollination, i.e., when the pollen and ovule (or microspores and megaspores) are from the same plant or genetically identical plants).

The term “introgression” refers to the transmission of a desired allele of a genetic locus from one genetic background to another. For example, introgression of a desired allele at a specified locus can be transmitted to at least one progeny plant via a sexual cross between two parent plants, where at least one of the parent plants has the desired allele within its genome. Alternatively, for example, transmission of an allele can occur by recombination between two donor genomes, e.g., in a fused protoplast, where at least one of the donor protoplasts has the desired allele in its genome. The desired allele can be, e.g., a transgene, a modified (mutated or edited) native allele, or a selected allele of a marker or QTL.

The term “isoline” is a comparative term, and references organisms that are genetically identical, but differ in treatment. In one example, two genetically identical maize plant embryos may be separated into two different groups, one receiving a treatment (such as the introduction of a CRISPR-Cas effector endonuclease) and one control that does not receive such treatment. Any phenotypic differences between the two groups may thus be attributed solely to the treatment and not to any inherency of the plant's endogenous genetic makeup.

“Introducing” is intended to mean presenting to a target, such as a cell or organism, a polynucleotide or polypeptide or polynucleotide-protein complex, in such a manner that the component(s) gains access to the interior of a cell of the organism or to the cell itself.

A “polynucleotide of interest” includes any nucleotide sequence encoding a protein or polypeptide that improves desirability of crops, i.e. a trait of agronomic interest. Polynucleotides of interest include, but are not limited to: polynucleotides encoding important traits for agronomics, herbicide-resistance, insecticidal resistance, disease resistance, nematode resistance, herbicide resistance, microbial resistance, fungal resistance, viral resistance, fertility or sterility, grain characteristics, commercial products, phenotypic marker, or any other trait of agronomic or commercial importance. A polynucleotide of interest may additionally be utilized in either the sense or anti-sense orientation. Further, more than one polynucleotide of interest may be utilized together, or “stacked”, to provide additional benefit.

A “complex trait locus” includes a genomic locus that has multiple transgenes genetically linked to each other.

The compositions and methods herein may provide for an improved “agronomic trait” or “trait of agronomic importance” or “trait of agronomic interest” to a plant, which may include, but not be limited to, the following: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, as compared to an isoline plant not comprising a modification derived from the methods or compositions herein.

“Agronomic trait potential” is intended to mean a capability of a plant element for exhibiting a phenotype, preferably an improved agronomic trait, at some point during its life cycle, or conveying said phenotype to another plant element with which it is associated in the same plant.

The terms “decreased,” “fewer,” “slower” and “increased” “faster” “enhanced” “greater” as used herein refers to a decrease or increase in a characteristic of the modified plant element or resulting plant compared to an unmodified plant element or resulting plant. For example, a decrease in a characteristic may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400%) or more lower than the untreated control and an increase may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400% or more higher than the untreated control.

As used herein, the term “before”, in reference to a sequence position, refers to an occurrence of one sequence upstream, or 5′, to another sequence.

The meaning of abbreviations is as follows: “sec” means second(s), “min” means minute(s), “h” means hour(s), “d” means day(s), “μL” means microliter(s), “mL” means milliliter(s), “L” means liter(s), “μM” means micromolar, “mM” means millimolar, “M” means molar, “mmol” means millimole(s), “μmole” or “umole” mean micromole(s), “g” means gram(s), “μg” or “ug” means microgram(s), “ng” means nanogram(s), “U” means unit(s), “bp” means base pair(s) and “kb” means kilobase(s).

### Classification of CRISPR-Cas Systems

CRISPR-Cas systems have been classified according to sequence and structural analysis of components. Multiple CRISPR/Cas systems have been described including Class 1 systems, with multisubunit effector complexes (comprising type I, type III, and type IV), and Class 2 systems, with single protein effectors (comprising type II, type V, and type VI) (Makarova et al. 2015, Nature Reviews Microbiology Vol. 13:1-15; Zetsche et al. 2015, Cell 163, 1-13; Shmakov et al. 2015, Molecular Cell 60, 1-13; Haft et al. 2005, Computational Biology, PLoS Comput Biol 1(6):e60; and Koonin et al. 2017, Curr Opinion Microbiology 37:67-78).

A CRISPR-Cas system comprises, at a minimum, a CRISPR RNA (crRNA) molecule and at least one CRISPR-associated (Cas) protein to form crRNA ribonucleoprotein (crRNP) effector complexes. CRISPR-Cas loci comprise an array of identical repeats interspersed with DNA-targeting spacers that encode the crRNA components and an operon-like unit of cas genes encoding the Cas protein components. The resulting ribonucleoprotein complex recognizes a polynucleotide in a sequence-specific manner (Jore et al. 2011, Nature Structural & Molecular Biology 18, 529-536). The crRNA serves as a guide RNA for sequence specific binding of the effector (protein or complex) to double strand DNA sequences, by forming base pairs with the complementary DNA strand while displacing the noncomplementary strand to form a so-called R-loop. (Jore et al. 2011).

RNA transcripts of CRISPR loci (pre-crRNA) are cleaved specifically in the repeat sequences by CRISPR associated (Cas) endoribonucleases in type I and type III systems or by RNase III in type II systems. The number of CRISPR-associated genes at a given CRISPR locus can vary between species.

Different cas genes that encode proteins with different domains are present in different CRISPR systems. The cas operon comprises genes that encode for one or more effector endonucleases, as well as other Cas proteins. Protein subunits include those described in Makarova et al. 2011, Nat Rev Microbiol. 2011 9(6):467-477; Makarova et al. 2015, Nature Reviews Microbiology Vol. 13:1-15; and Koonin et al. 2017, Current Opinion Microbiology 37:67-78). The types of domains include those involved in Expression (pre-crRNA processing, for example Cas 6 or RNaseIII), Interference (including an effector module for crRNA and target binding, as well as domain(s) for target cleavage), Adaptation (spacer insertion, for example Cas1 or Cas2), and Ancillary (regulation or helper or unknown function). Some domains may serve more than one purpose, for example Cas9 comprises domains for endonuclease functionality as well as for target cleavage, among others.

The Cas endonuclease is guided by a single CRISPR RNA (crRNA) through direct RNA-DNA base-pairing to recognize a DNA target site that is in close vicinity to a protospacer adjacent motif (PAM) (Jore et al. 2011, supra, Westra et al. 2012, Molecular Cell 46:595-605, and Sinkunas et al. 2013, EMBO J. 32:385-394).

### Class I CRISPR-Cas Systems

Class I CRISPR-Cas systems comprise Types I, III, and IV. A characteristic feature of Class I systems is the presence of an effector endonuclease complex instead of a single protein. A Cascade complex comprises an RNA recognition motif (RRM) and a nucleic acid-binding domain that is the core fold of the diverse RAMP (Repeat-Associated Mysterious Proteins) protein superfamily (Makarova et al. 2013, Biochem Soc Trans 41, 1392-1400; Makarova et al. 2015, Nature Reviews Microbiology Vol. 13:1-15). RAMP protein subunits include Cas5 and Cas7 (which comprise the skeleton of the crRNA-effector complex), wherein the Cas5 subunit binds the 5′ handle of the crRNA and interacts with the large subunit, and often includes Cas6 which is loosely associated with the effector complex and typically functions as the repeat-specific RNase in the pre-crRNA processing (Charpentier et al. FEMS Microbiol Rev 2015, 39:428-441; Niewoehner et al. 2016 RNA, 22:318-329).

Type I CRISPR-Cas systems comprise a complex of effector proteins, termed Cascade (CRISPR-associated complex for antiviral defense) comprising at a minimum Cas5 and Cas7. The effector complex functions together with a single CRISPR RNA (crRNA) and Cas3 to defend against invading viral DNA (Brouns, S. J. J. et al. Science 321:960-964; Makarova et al. 2015, Nature Reviews Microbiology Vol. 13:1-15). Type I CRISPR-Cas loci comprise the signature gene cas3 (or a variant cas3′ or cas3″), which encodes a metal-dependent nuclease that possesses a single-stranded DNA (ssDNA)-stimulated superfamily 2 helicase with a demonstrated capacity to unwind double stranded DNA (dsDNA) and RNA-DNA duplexes (Makarova et al. 2015, Nature Reviews; Microbiology Vol. 13:1-15). Following target recognition, the Cas3 endonuclease is recruited to the Cascade-crRNA-target DNA complex to cleave and degrade the DNA target (Westra, E. R. et al. (2012) Molecular Cell 46:595-605, Sinkunas, T. et al. (2011) EMBO J. 30:1335-1342, and Sinkunas, T. et al. (2013) EMBO J. 32:385-394). In some type I systems, Cas6 can be the active endonuclease that is responsible for crRNA processing, and Cas5 and Cas7 function as non-catalytic RNA-binding proteins; although in type I-C systems, crRNA processing can be catalyzed by Cas5 (Makarova et al. 2015, Nature Reviews Microbiology Vol. 13:1-15). Type I systems are divided into seven subtypes (Makarova et al. 2011, Nat Rev Microbiol. 2011 9(6):467-477; Koonin et al. 2017, Curr Opinion Microbiology 37:67-78). A modified type I CRISPR-associated complex for adaptive antiviral defense (Cascade) comprising at least the protein subunits Cas7, Cas5 and Cas6, wherein one of these subunits is synthetically fused to a Cas3 endonuclease or a modified restriction endonuclease, FokI, have been described (WO2013098244 published Jul. 4, 2013).

Type III CRISPR-Cas systems, comprising a plurality of cas7 genes, target either ssRNA or ssDNA, and function as either an RNase as well as a target RNA-activated DNA nuclease (Tamulaitis et al. 2017 Trends in Microbiology 25(10)49-61,). Csm (Type III-A) and Cmr (Type III-B) complexes function as RNA-activated single-stranded (ss) DNases that couple the target RNA binding/cleavage with ssDNA degradation. Upon foreign DNA infection, the CRISPR RNA (crRNA)-guided binding of the Csm or Cmr complex to the emerging transcript recruits Cas10 DNase to the actively transcribed phage DNA, resulting in degradation of both the transcript and phage DNA, but not the host DNA. The Cas10 HD-domain is responsible for the ssDNase activity, and Csm3/Cmr4 subunits are responsible for the endoribonuclease activity of the Csm/Cmr complex. The 3′-flanking sequence of the target RNA is critical for the ssDNase activity of Csm/Cmr: the base pairing with the 5′-handle of crRNA protects host DNA from degradation.

Type IV systems, although comprising typical type I cas5 and cas7 domains in addition to a cas8-like domain, may lack the CRISPR array that is characteristic of most other CRISPR-Cas systems.

### Class II CRISPR-Cas Systems

Class II CRISPR-Cas systems comprise Types II, V, and VI. A characteristic feature of Class II systems is the presence of a single Cas effector protein instead of an effector complex. Types II and V Cas proteins comprise an RuvC endonuclease domain that adopts the RNase H fold.

Type II CRISPR/Cas systems employ a crRNA and tracrRNA (trans-activating CRISPR RNA) to guide the Cas endonuclease to its DNA target. The crRNA comprises a spacer region complementary to one strand of the double strand DNA target and a region that base pairs with the tracrRNA (trans-activating CRISPR RNA) forming an RNA duplex that directs the Cas endonuclease to cleave the DNA target, leaving a blunt end. Spacers are acquired through a not fully understood process involving Cas1 and Cas2 proteins. Type II CRISPR/Cas loci typically comprise cas1 and cas2 genes in addition to the cas9 gene (Chylinski et al. 2013, RNA Biology 10:726-737; Makarova et al. 2015, Nature Reviews Microbiology Vol. 13:1-15). Type II CRISR-Cas loci can encode a tracrRNA, which is partially complementary to the repeats within the respective CRISPR array, and can comprise other proteins such as Csn1 and Csn2. The presence of cas9 in the vicinity of cas1 and cas2 genes is the hallmark of type II loci (Makarova et al. 2015, Nature Reviews Microbiology Vol. 13:1-15).

Type V CRISPR/Cas systems comprise a single Cas endonuclease, including Cpf1 (Casl2) (Koonin et al. 2017 Curr Opinion Microbiology 37:67-78,), that is an active RNA-guided endonuclease that does not necessarily require the additional trans-activating CRISPR (tracr) RNA for target cleavage, unlike Cas9.

Type VI CRISPR-Cas systems comprise a cas13 gene that encodes a nuclease with two HEPN (Higher Eukaryotes and Prokaryotes Nucleotide-binding) domains but no HNH or RuvC domains, and are not dependent upon tracrRNA activity. The majority of HEPN domains comprise conserved motifs that constitute a metal-independent endoRNase active site (Anantharam et al. 2013 Biol Direct 8:15,). Because of this feature, it is thought that type VI systems act on RNA targets instead of the DNA targets that are common to other CRISPR-Cas systems.

### Novel Cas Endonuclease CRISPR-Cas Systems

Disclosed herein is a novel CRISPR-Cas system, components thereof, and methods of using said components. The system comprises a novel Cas effector protein, Cas endonuclease.

The novel CRISPR-Cas system components described herein may comprise one or more subunits from different Cas systems, subunits derived or modified from more than one different bacterial or archaeal prokaryote, and/or synthetic or engineered components.

Described herein is a newly identified CRISPR-Cas system comprising novel arrangements of cas genes. Further described are novel cas genes and proteins.

One feature of the novel Cas endonuclease system is the locus architecture, comprising an endonuclease gene upstream of a CRISPR array, but not comprising a cas1 gene, a cas2 gene, or a cas4 gene.

### CRISPR-Cas System Components

### Cas Proteins

A number of proteins may be encoded in the CRISPR cas operon, including those involved in adaptation (spacer insertion), interference (effector module target binding, target nicking or cleavage—e.g. endonuclease activity), expression (pre-crRNA processing), regulation, or other.

Two proteins, Cas1 and Cas2, are conserved among many CRISPR systems (for example, as described in Koonin et al. 2017, Curr Opinion Microbiology 37:67-78). Cas1 is a metal-dependent DNA-specific endonuclease that produces double-stranded DNA fragments. In some systems Cas1 forms a stable complex with Cas2, which is essential to spacer acquisition and insertion for CRISPR systems (Nunez et al. 2014, Nature Str Mol Biol 21:528-534).

A number of other proteins have been identified across different systems, including Cas4 (which may have similarity to a RecB nuclease) and is thought to play a role in the capture of new viral DNA sequences for incorporation into the CRISPR array (Zhang et al. 2012, PLOS One 7(10):e47232).

Some proteins may encompass a plurality of functions. For example, Cas9, the signature protein of Class 2 type II systems, has been demonstrated to be involved in pre-crRNA processing, target binding, as well as target cleavage.

In some native systems, such as the Cas endonuclease CRISPR system from Syntrophomonas palmitatica, no genes encoding Cas1, Cas2, or Cas4 proteins were detect near the endonuclease gene.

### Cas Endonucleases and Effectors

Endonucleases are enzymes that cleave the phosphodiester bond within a polynucleotide chain, and include restriction endonucleases that cleave DNA at specific sites without damaging the bases. Examples of endonucleases include restriction endonucleases, meganucleases, TAL effector nucleases (TALENs), zinc finger nucleases, and Cas (CRISPR-associated) effector endonucleases.

Cas endonucleases, either as single effector proteins or in an effector complex with other components, unwind the DNA duplex at the target sequence and optionally cleave at least one DNA strand, as mediated by recognition of the target sequence by a polynucleotide (such as, but not limited to, a crRNA or guide RNA) that is in complex with the Cas effector protein. Such recognition and cutting of a target sequence by a Cas endonuclease typically occurs if the correct protospacer-adjacent motif (PAM) is located at or adjacent to the 3′ end of the DNA target sequence. Alternatively, a Cas endonuclease herein may lack DNA cleavage or nicking activity, but can still specifically bind to a DNA target sequence when complexed with a suitable RNA component. (See also U.S. Patent Application US20150082478 published 19 Mar. 2015 and US20150059010 published 26 Feb. 2015).

Cas endonucleases may occur as individual effectors (Class 2 CRISPR systems) or as part of larger effector complexes (Class I CRISPR systems).

Cas endonucleases that have been described include, but are not limited to, for example: Cas3 (a feature of Class 1 type I systems), Cas9 (a feature of Class 2 type II systems) and Cas12 (Cpf1) (a feature of Class 2 type V systems).

Cas3 (and its variants Cas3′ and Cas3″) functions as a single-stranded DNA nuclease (HD domain) and an ATP-dependent helicase. A variant of the Cas3 endonuclease can be obtained by disabling the functional activity of one or both domains of the Cas3 endonuclease poly peptide. Disabling the ATPase dependent helicase activity (by deletion, knockout of the Cas3-helicase domain, or through mutagenesis of critical residues or by assembling the reaction in the absence of ATP as described previously (Sinkunas, T. et al. 2013, EMBO J. 32:385-394) can convert the cleavage ready Cascade comprising the modified Cas3 endonuclease into a nickase (as the HD domain is still functional). Disabling the HD endonuclease activity can be accomplished by any method known in the art, such as but not limited to, mutagenesis of critical residues of the HD domain, can convert the cleavage ready Cascade comprising the modified Cas3 endonuclease into a helicase. Disabling the both the Cas helicase and Cas3 HD endonuclease activity can be accomplished by any method known in the art, such as but not limited to, mutagenesis of critical residues of both the helicase and HD domains, can convert the cleavage ready Cascade comprising the modified Cas3 endonuclease into a binder protein that binds to a target sequence.

Cas9 (formerly referred to as Cas5, Csn1, or Csx12) is a Cas endonuclease that forms a complex with a crNucleotide and a tracrNucleotide, or with a single guide polynucleotide, for specifically recognizing and cleaving all or part of a DNA target sequence. Cas9 recognizes a 3′ GC-rich PAM sequence on the target dsDNA. A Cas9 protein comprises a RuvC nuclease with an HNH (H-N-H) nuclease adjacent to the RuvC-II domain. The RuvC nuclease and HNH nuclease each can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al. 2013, Cell 157:1262-1278). Cas9 endonucleases are typically derived from a type II CRISPR system, which includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA (Makarova et al. 2015, Nature Reviews Microbiology Vol. 13:1-15).

Cas12 (formerly referred to as Cpf1, and variants c2c1, c2c3, CasX, and CasY) comprise an RuvC nuclease domain and produced staggered, 5′ overhangs on the dsDNA target. Some variants do not require a tracrRNA, unlike the functionality of Cas9. Cas12 and its variants recognize a 5′ AT-rich PAM sequence on the target dsDNA. An insert domain, called Nuc, of the Casl2a protein has been demonstrated to be responsible for target strand cleavage (Yamano et al. 2016, Cell 165:949-962). Additional mutation studies in other Cas12 proteins demonstrated the Nuc domain contributes to guide and target binding, with the RuvC domain responsible for cleavage (Swarts et al. Mol Cell 2017, 66:221-233 e224).

Cas endonucleases and effector proteins can be used for targeted genome editing (via simplex and multiplex double-strand breaks and nicks) and targeted genome regulation (via tethering of epigenetic effector domains to either the Cas protein or sgRNA. A Cas endonuclease can also be engineered to function as an RNA-guided recombinase, and via RNA tethers could serve as a scaffold for the assembly of multiprotein and nucleic acid complexes (Mali et al. 2013, Nature Methods Vol. 10:957-963).

### Cas Endonucleases and Variants Thereof

A Cas endonuclease, or a functional variant thereof, is defined as a functional RNA-guided, PAM-dependent dsDNA cleavage protein of fewer than about 500 amino acids, comprising: a C-terminal RuvC catalytic domain split into three subdomains and further comprising bridge-helix and one or more Zinc finger motif(s); and an N-terminal Rec subunit with a helical bundle, WED wedge-like (or “Oligonucleotide Binding Domain”, OBD) domain, and, optionally, a Zinc finger motif.

The novel Cas endonuclease variant proteins disclosed herein include effector proteins (endonucleases). The Wild Type (WT) Cas endonuclease protein requires a PAM sequence of N(T>W>C)TTC at or near the target site of the target double-stranded polydeoxyribonucleotide.

Certain functional variants of Cas endonuclease disclosed herein are capable of double-strand break or single-strand nick activity, which activity may be less than the activity of the WT Cas endonuclease, approximately the same, or of even greater activity. Different levels of activity may have different uses according to the practitioner's desires. A Cas endonuclease, or functional variant, comprises, when aligned to SEQ ID NO:14 or SEQ ID NO:48, relative to the amino acid position numbers of SEQ ID NO:14 or SEQ ID NO:48, (i) Glutamate at position 34, Alanine at position 36, Serine at position 45, Aspartate at position 54, Serine at position 77, Glutamine at position 85 and (II) the following amino acid motifs: GxxxG starting at amino acid position 226; ExL starting at amino acid position 327, CxₙC starting at amino acid position 376; Cxₙ(C,H) starting at amino acid position 395; wherein X is any nucleotide and n is any number. In some aspects, a functional variant is one that does not have an Phenylalanine at position 38, Alanine (A) at position 40, Histidine at position 79, Glutamate at relative position 81, Alanine at relative position 87, Threonine at relative position 335, Cysteine at position 409, Glutamate at position 421, Lysine at position 467, or Glutamate at position 468, wherein each of these refers to a position relative to SEQ ID NO:14 or 48 in an alignment with SEQ ID NO:14 or 48. In some aspects, a functional variant is one that has, when aligned to SEQ ID NO:14 or SEQ ID NO:48, relative to the amino acid position numbers of SEQ ID NO:14 or SEQ ID NO:48, Aspartate or Glutamate at position 38, Aspartate at position 79, Proline at position 120, Aspartate at position 149, Glycine at 226, Glycine at 230, Histidine at position 293, Serine at position 298, Phenylalanine at position 306, Glutamate at 329, Serine at position 313, Asparagine at position 325, Glutamate at position 327, Valine at position 338, Cysteine at 376, Cysteine at 379, Cysteine at 395, Cysteine at 398, Cysteine at position 406, Lysine or Arginine at position 409, Asparagine or Arginine at position 421, Proline at position 430, Arginine at position 467, or Proline at position 468.

A “functional fragment” of a Cas endonuclease variant endonuclease refers to a polynucleotide of fewer than 497 amino acids that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 450, or greater than 450 contiguous amino acids of any one of SEQ ID NOs:15-47; and comprises the ability to recognize, or bind, or nick a single strand of a double-stranded polynucleotide, or cleave both strands of a double-stranded polynucleotide, or any combination of the preceding.

RuvC domains have been demonstrated in the literature to encompass endonuclease functionality. A Cas endonuclease may be isolated or identified from a locus that comprises a Cas endonuclease gene encoding an effector protein, and an array comprising a plurality repeats.

Zinc finger motifs are domains that coordinate one or more zinc ions, usually through Cysteine and Histidine sidechains, to stabilize their fold. Zinc fingers are named for the pattern of Cysteine and Histidine residues that coordinate the zinc ion (e.g., C4 means a zinc ion is coordinated by four Cysteine residues; C3H means a zinc ion is coordinated by three Cysteine residues and one Histidine residue).

Cas endonuclease proteins comprise one or more Zinc Finger (ZFN) coordination motif(s) that may form a Zinc binding domain. Zinc Finger-like motifs can aid in target and non-target strand separation and loading of the guide RNA into the DNA target. Cas endonuclease proteins comprising one or more Zinc Finger motifs may provide additional stability to the ribonucleoprotein complex on the target polynucleotide. Cas endonuclease proteins comprise C4 or C3H zinc binding domains.

As used herein, a “domain” is synonymous with “motif”. For example, a zinc finger domain and zinc finger motif are used synonymously. Similarly, a zinc binding domain and zinc binding motif are used synonymously.

Cas endonucleases are RNA-guided endonucleases capable of binding to, and cleaving, a double-strand DNA target that comprises: (1) a sequence sharing homology with a nucleotide sequence of the guide RNA, and (2) a PAM sequence.

A Cas endonuclease is functional as a double-strand-break-inducing agent, and may also be a nickase, or a single-strand-break inducing agent. In some aspects, a catalytically inactive Cas endonuclease may be used to target or recruit to a target DNA sequence but not induce cleavage. In some aspects, a catalytically inactive Cas endonuclease protein may be used with a functional endonuclease, to cleave a target sequence. In some aspects, a catalytically inactive Cas endonuclease protein may be combined with a base editing molecule, such as a deaminase. In some aspects, a deaminase may be a cytidine deaminase. In some aspects, a deaminase may be an adenine deaminase. In some aspects, a deaminase may be ADAR-2.

A Cas endonuclease is further defined as an RNA-guided double-strand DNA cleavage protein that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 450, or greater than 450 contiguous amino acids of any one of SEQ ID NOs:15-47 and that retains at least partial endonuclease activity.

The disclosed engineered Cas endonuclease, or inactivated engineered Cas polypeptide, may be encoded by a polynucleotide that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, between 500 and 550, at least 600, between 600 and 650, at least 650, between 650 and 700, at least 700, between 700 and 750, at least 750, between 750 and 800, at least 800, between 800 and 850, at least 850, between 850 and 900, at least 900, between 900 and 950, at least 950, between 950 and 1000, at least 1000, or even greater than 1000 contiguous nucleotides of any sequence encoding any one of SEQ ID NOs:15-47.

A Cas endonuclease, effector protein, or functional fragment thereof, for use in the disclosed methods, can be isolated from a native source, or from, a recombinant source where the genetically modified host cell is modified to express the nucleic acid sequence encoding the protein. Alternatively, the Cas protein can be produced using cell free protein expression systems, or be synthetically produced. Effector Cas nucleases may be isolated and introduced into a heterologous cell, or may be modified from its native form to exhibit a different type or magnitude of activity than what it would exhibit in its native source. Such modifications include but are not limited to fragments, variants, substitutions, deletions, and insertions. Cas endonuclease WT compositions are described in WO2020123887 published on 16 Jul. 2020.

Fragments and variants of Cas endonucleases and Cas endonuclease effector proteins can be obtained via methods such as site-directed mutagenesis and synthetic construction. Methods for measuring endonuclease activity are well known in the art such as, but not limiting to, WO2013166113 published 7 Nov. 2013, WO2016186953 published 24 Nov. 2016, and WO2016186946 published 24 Nov. 2016.

The Cas endonuclease can comprise a modified form of the Cas polypeptide. The modified form of the Cas polypeptide can include an amino acid change (e.g., deletion, insertion, or substitution) that reduces the naturally occurring nuclease activity of the Cas protein. For example, in some instances, the modified form of the Cas protein has less than 50%, less than 40%, less than 30%, less than 20%, less than 10%, less than 5%, or less than 1% of the nuclease activity of the corresponding wild-type Cas polypeptide (SEQ ID NO:48; see also US20140068797 published 6 Mar. 2014). In some cases, the modified form of the Cas polypeptide has no substantial nuclease activity and is referred to as catalytically “inactivated Cas” or “deactivated Cas (dCas).” An inactivated Cas/deactivated Cas includes a deactivated Cas endonuclease (dCas). A catalytically inactive Cas effector protein can be fused to a heterologous sequence to induce or modify activity.

A Cas endonuclease can be part of a fusion protein comprising one or more heterologous protein domains (e.g., 1, 2, 3, or more domains in addition to the Cas protein). Such a fusion protein may comprise any additional protein sequence, and optionally a linker sequence between any two domains, such as between Cas and a first heterologous domain. Examples of protein domains that may be fused to a Cas protein herein include, without limitation, epitope tags (e.g., histidine [His], V5, FLAG, influenza hemagglutinin [HA], myc, VSV-G, thioredoxin [Trx]), reporters (e.g., glutathione-5-transferase [GST], horseradish peroxidase [HRP], chloramphenicol acetyltransferase [CAT], beta-galactosidase, beta-glucuronidase [GUS], luciferase, green fluorescent protein [GFP], HcRed, DsRed, cyan fluorescent protein [CFP], yellow fluorescent protein [YFP], blue fluorescent protein [BFP]), and domains having one or more of the following activities: methylase activity, demethylase activity, transcription activation activity (e.g., VP16 or VP64), transcription repression activity, transcription release factor activity, histone modification activity, RNA cleavage activity and nucleic acid binding activity. A Cas protein can also be in fusion with a protein that binds DNA molecules or other molecules, such as maltose binding protein (MBP), S-tag, Lex A DNA binding domain (DBD), GAL4A DNA binding domain, and herpes simplex virus (HSV) VP16.

A catalytically active and/or inactive Cas endonuclease can be fused to a heterologous sequence (US20140068797 published 6 Mar. 2014). Suitable fusion partners include, but are not limited to, a polypeptide that provides an activity that indirectly increases transcription by acting directly on the target DNA or on a polypeptide (e.g., a histone or other DNA-binding protein) associated with the target DNA. Additional suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity. Further suitable fusion partners include, but are not limited to, a polypeptide that directly provides for increased transcription of the target nucleic acid (e.g., a transcription activator or a fragment thereof, a protein or fragment thereof that recruits a transcription activator, a small molecule/drug-responsive transcription regulator, etc.). A partially active or catalytically inactive Cas endonuclease can also be fused to another protein or domain, for example Clo51 or FokI nuclease, to generate double-strand breaks (Guilinger et al. Nature Biotechnology, volume 32, number 6, June 2014).

A catalytically active or inactive Cas protein, such as the Cas endonuclease protein described herein, can also be in fusion with a molecule that directs editing of single or multiple bases in a polynucleotide sequence, for example a site-specific deaminase that can change the identity of a nucleotide, for example from C·G to T·A or an A·T to G·C (Gaudelli et al. 2017, “Programmable base editing of A·T to G·C in genomic DNA without DNA cleavage.” Nature 551(7681):464-471; Nishida et al. 2016, “Targeted nucleotide editing using hybrid prokaryotic and vertebrate adaptive immune systems.” Science 353(6305):aaf8729; Komor et al. “Programmable editing of a target base in genomic DNA without double-stranded DNA cleavage.” Nature 533 (7603) (2016):420-4. A base editing fusion protein may comprise, for example, an active (double strand break creating), partially active (nickase) or deactivated (catalytically inactive) Cas endonuclease and a deaminase (such as, but not limited to, a cytidine deaminase, an adenine deaminase, APOBEC1, APOBEC3A, BE2, BE3, BE4, ABEs, or the like). Base edit repair inhibitors and glycosylase inhibitors (e.g., uracil glycosylase inhibitor (to prevent uracil removal)) are contemplated as other components of a base editing system, in some embodiments.

The Cas endonucleases described herein can be expressed and purified by methods known in the art, for example as described in WO/2016/186953 published 24 Nov. 2016.

Many Cas endonucleases have been described to date that can recognize specific PAM sequences (WO2016186953 published 24 Nov. 2016, WO2016186946 published 24 Nov. 2016, and Zetsche B et al. 2015. Cell 163, 1013) and cleave the target DNA at a specific position. It is understood that based on the methods and embodiments described herein utilizing a novel guided Cas system one skilled in the art can now tailor these methods such that they can utilize any guided endonuclease system.

A Cas effector protein can comprise a heterologous nuclear localization sequence (NLS). A heterologous NLS amino acid sequence herein may be of sufficient strength to drive accumulation of a Cas protein in a detectable amount in the nucleus of a yeast cell herein, for example. An NLS may comprise one (monopartite) or more (e.g., bipartite) short sequences (e.g., 2 to 20 residues) of basic, positively charged residues (e.g., lysine and/or arginine), and can be located anywhere in a Cas amino acid sequence but such that it is exposed on the protein surface. An NLS may be operably linked to the N-terminus or C-terminus of a Cas protein herein, for example. Two or more NLS sequences can be linked to a Cas protein, for example, such as on both the N- and C-termini of a Cas protein. The Cas endonuclease gene can be operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region. Non-limiting examples of suitable NLS sequences herein include those disclosed in U.S. Pat. Nos. 6,660,830 and 7,309,576.

### Guide Polynucleotides

The guide polynucleotide enables target recognition, binding, and optionally cleavage by the Cas endonuclease, and can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA” or “gRNA” (US20150082478 published 19 Mar. 2015 and US20150059010 published 26 Feb. 2015). A guide polynucleotide may be engineered or synthetic.

The guide polynucleotide includes a chimeric non-naturally occurring guide RNA comprising regions that are not found together in nature (i.e., they are heterologous with each other). For example, a chimeric non-naturally occurring guide RNA comprising a first nucleotide sequence domain (referred to as Variable Targeting domain or VT domain) that can hybridize to a nucleotide sequence in a target DNA, linked to a second nucleotide sequence that can recognize the Cas endonuclease, such that the first and second nucleotide sequence are not found linked together in nature.

The guide polynucleotide can be a double molecule (also referred to as duplex guide polynucleotide) comprising a crNucleotide sequence (such as a crRNA) and a tracrNucleotide (such as a tracrRNA) sequence. In some cases, there is a linker polynucleotide that connects the crRNA and tracrRNA to form a single guide, for example an sgRNA.

The crNucleotide includes a first nucleotide sequence domain (referred to as Variable Targeting domain or VT domain) that can hybridize to a nucleotide sequence in a target DNA and a second nucleotide sequence (also referred to as a tracr mate sequence) that is part of a Cas endonuclease recognition (CER) domain. The tracr mate sequence can hybridized to a tracrNucleotide along a region of complementarity and together form the Cas endonuclease recognition domain or CER domain. The CER domain is capable of interacting with a Cas endonuclease polypeptide. The crNucleotide and the tracrNucleotide of the duplex guide polynucleotide can be RNA, DNA, and/or RNA-DNA-combination sequences. In some embodiments, the crNucleotide molecule of the duplex guide polynucleotide is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the crRNA naturally occurring in Bacteria and Archaea. The size of the fragment of the crRNA naturally occurring in Bacteria and Archaea that can be present in a crNucleotide disclosed herein can range from, but is not limited to, 2, 3, 4, 5, 6, 7, 8, 9, 10, 11, 12, 13, 14, 15, 16, 17, 18, 19, 20 or more nucleotides.

In some embodiments the tracrNucleotide is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides. In one embodiment, the RNA that guides the RNA/Cas9 endonuclease complex is a duplexed RNA comprising a duplex crRNA-tracrRNA. The tracrRNA (trans-activating CRISPR RNA) comprises, in the 5′-to-3′ direction, (i) a sequence that anneals with the repeat region of CRISPR type II crRNA and (ii) a stem loop-comprising portion (Deltcheva et al. Nature 471:602-607). The duplex guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double-strand break) into the target site. (US20150082478 published 19 Mar. 2015 and US20150059010 published 26 Feb. 2015).

In one aspect, the guide polynucleotide is a guide polynucleotide capable of forming a PGEN as described herein, wherein said guide polynucleotide comprises a first nucleotide sequence domain that is complementary to a nucleotide sequence in a target DNA, and a second nucleotide sequence domain that interacts with the Cas polypeptide disclosed herein.

In one aspect, the guide polynucleotide is a guide polynucleotide described herein, wherein the first nucleotide sequence and the second nucleotide sequence domain is selected from the group consisting of a DNA sequence, a RNA sequence, and a combination thereof.

In one aspect, the guide polynucleotide is a guide polynucleotide described herein, wherein the first nucleotide sequence and the second nucleotide sequence domain is selected from the group consisting of RNA backbone modifications that enhance stability, DNA backbone modifications that enhance stability, and a combination thereof (see Kanasty et al. 2013, Common RNA-backbone modifications, Nature Materials 12:976-977; US20150082478 published 19 Mar. 2015 and US20150059010 published 26 Feb. 2015)

The guide RNA includes a dual molecule comprising a chimeric non-naturally occurring crRNA linked to at least one tracrRNA. A chimeric non-naturally occurring crRNA includes a crRNA that comprises regions that are not found together in nature (i.e., they are heterologous with each other. For example, a crRNA comprising a first nucleotide sequence domain (referred to as Variable Targeting domain or VT domain) that can hybridize to a nucleotide sequence in a target DNA, linked to a second nucleotide sequence (also referred to as a tracr mate sequence) such that the first and second sequence are not found linked together in nature.

The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide.

The VT domain and/or the CER domain of a single guide polynucleotide can comprise a RNA sequence, a DNA sequence, or a RNA-DNA-combination sequence. The single guide polynucleotide being comprised of sequences from the crNucleotide and the tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double-strand break) the target site. (US20150082478 published 19 Mar. 2015 and US20150059010 published 26 Feb. 2015).

A chimeric non-naturally occurring single guide RNA (sgRNA) includes a sgRNA that comprises regions that are not found together in nature (i.e., they are heterologous with each other. For example, a sgRNA comprising a first nucleotide sequence domain (referred to as Variable Targeting domain or VT domain) that can hybridize to a nucleotide sequence in a target DNA linked to a second nucleotide sequence (also referred to as a tracr mate sequence) that are not found linked together in nature.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide (also referred to as “loop”) can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another embodiment, the nucleotide sequence linking the crNucleotide and the tracrNucleotide of a single guide polynucleotide can comprise a tetraloop sequence, such as, but not limiting to a GAAA tetraloop sequence.

The guide polynucleotide can be produced by any method known in the art, including chemically synthesizing guide polynucleotides (such as but not limiting to Hendel et al. 2015, Nature Biotechnology 33, 985-989), in vitro generated guide polynucleotides, and/or self-splicing guide RNAs (such as but not limited to Xie et al. 2015, PNAS 112:3570-3575).

The guide polypeptide can be used in conjunction with any of the engineered Cas polypeptides disclosed herein, including any one of the engineered Cas polypeptides or endonucleases disclosed in the Summary herein at paragraphs [0007] to [0027]. See also the discussion of Guide Polynucleotide/Cas Endonuclease Complexes herein.

### Protospacer Adjacent Motif (PAM)

A “protospacer adjacent motif” (PAM) herein refers to a short nucleotide sequence adjacent to a target sequence (protospacer) that can be recognized (targeted) by a guide polynucleotide/Cas endonuclease system. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

A “randomized PAM” and “randomized protospacer adjacent motif” are used interchangeably herein, and refer to a random DNA sequence adjacent to a target sequence (protospacer) that is recognized (targeted) by a guide polynucleotide/Cas endonuclease system. The randomized PAM sequence can be of any length but is typically 1, 2, 3, 4, 5, 6, 7, 8, 9, 10, 11, 12, 13, 14, 15, 16, 17, 18, 19 or 20 nucleotides long. A randomized nucleotide includes anyone of the nucleotides A, C, G or T.

### Guide Polynucleotide/Cas Endonuclease Complexes

A guide polynucleotide/Cas endonuclease complex described herein is capable of recognizing, binding to, and optionally nicking, unwinding, or cleaving all or part of a target sequence.

A guide polynucleotide/Cas endonuclease complex that can cleave both strands of a DNA target sequence typically comprises a Cas protein that has all of its endonuclease domains in a functional state (e.g., wild type endonuclease domains or variants thereof retaining some or all activity in each endonuclease domain). Thus, a wild-type Cas protein (e.g., a Cas protein disclosed herein), or a variant thereof retaining some or all activity in each endonuclease domain of the Cas protein, is a suitable example of a Cas endonuclease that can cleave both strands of a DNA target sequence.

A guide polynucleotide/Cas endonuclease complex that can cleave one strand of a DNA target sequence can be characterized herein as having nickase activity (e.g., partial cleaving capability). A Cas nickase typically comprises one functional endonuclease domain that allows the Cas to cleave only one strand (i.e., make a nick) of a DNA target sequence. For example, a Cas9 nickase may comprise (i) a mutant, dysfunctional RuvC domain and (ii) a functional HNH domain (e.g., wild type HNH domain). As another example, a Cas9 nickase may comprise (i) a functional RuvC domain (e.g., wild type RuvC domain) and (ii) a mutant, dysfunctional HNH domain. Non-limiting examples of Cas9 nickases suitable for use herein are disclosed in US20140189896 published on 3 Jul. 2014. A pair of Cas nickases can be used to increase the specificity of DNA targeting. In general, this can be done by providing two Cas nickases that, by virtue of being associated with RNA components with different guide sequences, target and nick nearby DNA sequences on opposite strands in the region for desired targeting. Such nearby cleavage of each DNA strand creates a double-strand break (i.e., a DSB with single-stranded overhangs), which is then recognized as a substrate for non-homologous-end-joining, NHEJ (prone to imperfect repair leading to mutations) or homologous recombination, HR. Each nick in these embodiments can be at least about 5, between 5 and 10, at least 10, between 10 and 15, at least 15, between 15 and 20, at least 20, between 20 and 30, at least 30, between 30 and 40, at least 40, between 40 and 50, at least 50, between 50 and 60, at least 60, between 60 and 70, at least 70, between 70 and 80, at least 80, between 80 and 90, at least 90, between 90 and 100, or 100 or greater (or any integer between 5 and 100) bases apart from each other, for example. One or two Cas nickase proteins herein can be used in a Cas nickase pair. For example, a Cas9 nickase with a mutant RuvC domain, but functioning HNH domain (i.e., Cas9 HNH+/RuvC−), can be used (e.g., Streptococcus pyogenes Cas9 HNH+/RuvC−). Each Cas9 nickase (e.g., Cas9 HNH+/RuvC−) can be directed to specific DNA sites nearby each other (up to 100 base pairs apart) by using suitable RNA components herein with guide RNA sequences targeting each nickase to each specific DNA site.

A guide polynucleotide/Cas endonuclease complex in certain embodiments can bind to a DNA target site sequence, but does not cleave any strand at the target site sequence. Such a complex may comprise a Cas protein in which all of its nuclease domains are mutant, dysfunctional. For example, a Cas9 protein that can bind to a DNA target site sequence, but does not cleave any strand at the target site sequence, may comprise both a mutant, dysfunctional RuvC domain and a mutant, dysfunctional HNH domain. A Cas protein herein that binds, but does not cleave, a target DNA sequence can be used to modulate gene expression, for example, in which case the Cas protein could be fused with a transcription factor (or portion thereof) (e.g., a repressor or activator, such as any of those disclosed herein).

In one aspect, the guide polynucleotide/Cas endonuclease complex (PGEN) described herein is a PGEN, wherein said Cas endonuclease is optionally covalently or non-covalently linked, or assembled to at least one protein subunit, or functional fragment thereof.

In one embodiment of the disclosure, the guide polynucleotide/Cas endonuclease complex is a guide polynucleotide/Cas endonuclease complex (PGEN) comprising at least one guide polynucleotide and at least one Cas endonuclease polypeptide, wherein said Cas endonuclease polypeptide comprises at least one protein subunit, or a functional fragment thereof, wherein said guide polynucleotide is a chimeric non-naturally occurring guide polynucleotide, wherein said guide polynucleotide/Cas endonuclease complex is capable of recognizing, binding to, and optionally nicking, unwinding, or cleaving all or part of a target sequence.

The Cas effector protein can be a Cas endonuclease effector protein as disclosed herein.

In one embodiment of the disclosure, the guide polynucleotide/Cas effector complex is a guide polynucleotide/Cas effector protein complex (PGEN) comprising at least one guide polynucleotide and a Cas endonuclease effector protein, wherein said guide polynucleotide/Cas effector protein complex is capable of recognizing, binding to, and optionally nicking, unwinding, or cleaving all or part of a target sequence.

The PGEN can be a guide polynucleotide/Cas effector protein complex, wherein said Cas effector protein is a Cas polypeptide disclosed herein that further comprises one copy or multiple copies of at least one protein subunit, or a functional fragment thereof. In some embodiments, said protein subunit is selected from the group consisting of a Cas1 protein subunit, a Cas2 protein subunit, a Cas4 protein subunit, and any combination thereof. The PGEN can be a guide polynucleotide/Cas effector protein complex, wherein said Cas effector protein further comprises at least two different protein subunits of selected from the group consisting of a Cas1, Cas2, and Cas4.

The PGEN can be a guide polynucleotide/Cas effector protein complex, wherein said Cas effector protein further comprises at least three different protein subunits, or functional fragments thereof, selected from the group consisting of Cas1, Cas2, and one additional Cas protein, optionally comprising Cas4.

In one aspect, the guide polynucleotide/Cas effector protein complex (PGEN) described herein is a PGEN, wherein said Cas effector protein is a Cas polypeptide disclosed herein that is covalently or non-covalently linked to at least one protein subunit, or functional fragment thereof. The PGEN can be a guide polynucleotide/Cas effector protein complex, wherein said Cas effector protein polypeptide is covalently or non-covalently linked, or assembled to one copy or multiple copies of at least one protein subunit, or a functional fragment thereof, selected from the group consisting of a Cas1 protein subunit, a Cas2 protein subunit, a one additional Cas protein optionally comprising Cas4 protein subunit, and any combination thereof. The PGEN can be a guide polynucleotide/Cas effector protein complex, wherein said Cas effector protein is covalently or non-covalently linked or assembled to at least two different protein subunits selected from the group consisting of a Cas1, a Cas2, and one additional Cas protein, optionally comprising Cas4. The PGEN can be a guide polynucleotide/Cas effector protein complex, wherein said Cas effector protein is covalently or non-covalently linked to at least three different protein subunits, or functional fragments thereof, selected from the group consisting of a Cas1, a Cas2, and one additional Cas protein, optionally comprising Cas4, and any combination thereof.

Any component of the guide polynucleotide/Cas effector protein complex, the guide polynucleotide/Cas effector protein complex itself, as well as the polynucleotide modification template(s) and/or donor DNA(s), can be introduced into a heterologous cell or organism by any method known in the art.

### Recombinant Constructs for Transformation of Cells

The disclosed guide polynucleotides, Cas polypeptides or Cas endonucleases, polynucleotide modification templates, donor DNAs, guide polynucleotide/Cas endonuclease systems disclosed herein, and any one combination thereof, optionally further comprising one or more polynucleotide(s) of interest, can be introduced into a cell. Cells include, but are not limited to, human, non-human, animal, bacterial, fungal, insect, yeast, non-conventional yeast, and plant cells as well as plants and seeds produced by the methods described herein.

Standard recombinant DNA and molecular cloning techniques used herein are well known in the art and are described more fully in Sambrook et al. Molecular Cloning: A Laboratory Manual; Cold Spring Harbor Laboratory: Cold Spring Harbor, NY (1989). Transformation methods are well known to those skilled in the art and are described infra.

Vectors and constructs include circular plasmids, and linear polynucleotides, comprising a polynucleotide of interest and optionally other components including linkers, adapters, regulatory or analysis. In some examples a recognition site and/or target site can be comprised within an intron, coding sequence, 5′ UTRs, 3′ UTRs, and/or regulatory regions.

### Components for Expression and Utilization of Novel CRISPR-Cas Systems in Prokaryotic and Eukaryotic Cells

The invention further provides expression constructs for expressing in a prokaryotic or eukaryotic cell/organism a guide RNA/Cas system that is capable of recognizing, binding to, and optionally nicking, unwinding, or cleaving all or part of a target sequence.

In one embodiment, the expression constructs of the disclosure comprise a promoter operably linked to a nucleotide sequence encoding a Cas gene (or plant optimized, including a Cas endonuclease gene described herein) and a promoter operably linked to a guide RNA of the present disclosure. The promoter is capable of driving expression of an operably linked nucleotide sequence in a prokaryotic or eukaryotic cell/organism.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

A method of expressing RNA components such as gRNA in eukaryotic cells for performing Cas9-mediated DNA targeting has been to use RNA polymerase III (Pol III) promoters, which allow for transcription of RNA with precisely defined, unmodified, 5′- and 3′-ends (DiCarlo et al. 2013, Nucleic Acids Res. 41(7):4336-4343; Ma et al. 2014, Mol. Ther. Nucleic Acids 3:e161). This strategy has been successfully applied in cells of several different species including maize and soybean (US20150082478 published 19 Mar. 2015). Methods for expressing RNA components that do not have a 5′ cap have been described (WO2016/025131 published 18 Feb. 2016).

Various methods and compositions can be employed to obtain a cell or organism having a polynucleotide of interest inserted in a target site for a Cas endonuclease. Such methods can employ homologous recombination (HR) to provide integration of the polynucleotide of interest at the target site. In one method described herein, a polynucleotide of interest is introduced into the organism cell via a donor DNA construct.

The donor DNA construct further comprises a first and a second region of homology that flank the polynucleotide of interest. The first and second regions of homology of the donor DNA share homology to a first and a second genomic region, respectively, present in or flanking the target site of the cell or organism genome.

The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al. 2013, Nature Methods Vol. 10:957-963).

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity at least of about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, between 98% and 99%, 99%, between 99% and 100%, or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al. (1989) Molecular Cloning: A Laboratory Manual, (Cold Spring Harbor Laboratory Press, NY); Current Protocols in Molecular Biology, Ausubel et al. Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some instances the regions of homology share significant sequence homology to the genomic sequence immediately flanking the target site, it is recognized that the regions of homology can be designed to have sufficient homology to regions that may be further 5′ or 3′ to the target site. The regions of homology can also have homology with a fragment of the target site along with downstream genomic regions

In one embodiment, the first region of homology further comprises a first fragment of the target site and the second region of homology comprises a second fragment of the target site, wherein the first and second fragments are dissimilar.

### Polynucleotides of Interest

Polynucleotides of interest are further described herein and include polynucleotides reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for genetic engineering will change accordingly.

General categories of polynucleotides of interest include, for example, genes of interest involved in information, such as zinc fingers, those involved in communication, such as kinases, and those involved in housekeeping, such as heat shock proteins. More specific polynucleotides of interest include, but are not limited to, genes involved in traits of agronomic interest such as but not limited to: crop yield, grain quality, crop nutrient content, starch and carbohydrate quality and quantity as well as those affecting kernel size, sucrose loading, protein quality and quantity, nitrogen fixation and/or utilization, fatty acid and oil composition, genes encoding proteins conferring resistance to abiotic stress (such as drought, nitrogen, temperature, salinity, toxic metals or trace elements, or those conferring resistance to toxins such as pesticides and herbicides), genes encoding proteins conferring resistance to biotic stress (such as attacks by fungi, viruses, bacteria, insects, and nematodes, and development of diseases associated with these organisms).

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389.

Polynucleotide sequences of interest may encode proteins involved in providing disease or pest resistance. By “disease resistance” or “pest resistance” is intended that the plants avoid the harmful symptoms that are the outcome of the plant-pathogen interactions. Pest resistance genes may encode resistance to pests that have great yield drag such as rootworm, cutworm, European Corn Borer, and the like. Disease resistance and insect resistance genes such as lysozymes or cecropins for antibacterial protection, or proteins such as defensins, glucanases or chitinases for antifungal protection, or Bacillus thuringiensis endotoxins, protease inhibitors, collagenases, lectins, or glycosidases for controlling nematodes or insects are all examples of useful gene products. Genes encoding disease resistance traits include detoxification genes, such as against fumonisin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like. Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); and the like.

An “herbicide resistance protein” or a protein resulting from expression of an “herbicide resistance-encoding nucleic acid molecule” includes proteins that confer upon a cell the ability to tolerate a higher concentration of an herbicide than cells that do not express the protein, or to tolerate a certain concentration of an herbicide for a longer period of time than cells that do not express the protein. Herbicide resistance traits may be introduced into plants by genes coding for resistance to herbicides that act to inhibit the action of acetolactate synthase (ALS, also referred to as acetohydroxyacid synthase, AHAS), in particular the sulfonylurea (UK.:sulphonylurea) type herbicides, genes coding for resistance to herbicides that act to inhibit the action of glutamine synthase, such as phosphinothricin or basta (e.g., the bar gene), glyphosate (e.g., the EPSP synthase gene and the GAT gene), HPPD inhibitors (e.g., the HPPD gene) or other such genes known in the art. See, for example, U.S. Pat. Nos. 7,626,077, 5,310,667, 5,866,775, 6,225,114, 6,248,876, 7,169,970, 6,867,293, and 9,187,762. The bar gene encodes resistance to the herbicide basta, the nptII gene encodes resistance to the antibiotics kanamycin and geneticin, and the ALS-gene mutants encode resistance to the herbicide chlorsulfuron.

Furthermore, it is recognized that the polynucleotide of interest may also comprise antisense sequences complementary to at least a portion of the messenger RNA (mRNA) for a targeted gene sequence of interest. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

In addition, the polynucleotide of interest may also be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity. See U.S. Pat. Nos. 5,283,184 and 5,034,323.

The polynucleotide of interest can also be a phenotypic marker. A phenotypic marker is screenable or a selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that comprises it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as 0-galactosidase, GUS; fluorescent proteins such as green fluorescent protein (GFP), cyan (CFP), yellow (YFP), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

Additional selectable markers include genes that confer resistance to herbicidal compounds, such as sulphonylureas, glufosinate ammonium, bromoxynil, imidazolinones, and 2,4-dichlorophenoxyacetate (2,4-D). See for example, Acetolactase synthase (ALS) for resistance to sulfonylureas, imidazolinones, triazolopyrimidine sulfonamides, pyrimidinylsalicylates and sulphonylaminocarbonyl-triazolinones (Shaner and Singh, 1997, Herbicide Activity: Toxicol Biochem Mol Biol 69-110); glyphosate resistant 5-enolpyruvylshikimate-3-phosphate (EPSPS) (Saroha et al. 1998, J. Plant Biochemistry & Biotechnology Vol 7:65-72);

Polynucleotides of interest includes genes that can be stacked or used in combination with other traits, such as but not limited to herbicide resistance or any other trait described herein. Polynucleotides of interest and/or traits can be stacked together in a complex trait locus as described in US20130263324 published 3 Oct. 2013 and in WO/2013/112686, published 1 Aug. 2013.

A polypeptide of interest includes any protein or polypeptide that is encoded by a polynucleotide of interest described herein.

Further provided are methods for identifying at least one plant cell, comprising in its genome, a polynucleotide of interest integrated at the target site. A variety of methods are available for identifying those plant cells with insertion into the genome at or near to the target site. Such methods can be viewed as directly analyzing a target sequence to detect any change in the target sequence, including but not limited to PCR methods, sequencing methods, nuclease digestion, Southern blots, and any combination thereof. See, for example, US20090133152 published 21 May 2009. The method also comprises recovering a plant from the plant cell comprising a polynucleotide of interest integrated into its genome. The plant may be sterile or fertile. It is recognized that any polynucleotide of interest can be provided, integrated into the plant genome at the target site, and expressed in a plant.

### Optimization of Sequences for Expression in Plants

Methods are available in the art for synthesizing plant-preferred genes. See, for example, U.S. Pat. Nos. 5,380,831, and 5,436,391, and Murray et al. (1989) Nucleic Acids Res. 17:477-498. Additional sequence modifications are known to enhance gene expression in a plant host. These include, for example, elimination of: one or more sequences encoding spurious polyadenylation signals, one or more exon-intron splice site signals, one or more transposon-like repeats, and other such well-characterized sequences that may be deleterious to gene expression. The G-C content of the sequence may be adjusted to levels average for a given plant host, as calculated by reference to known genes expressed in the host plant cell. When possible, the sequence is modified to avoid one or more predicted hairpin secondary mRNA structures. Thus, “a plant-optimized nucleotide sequence” of the present disclosure comprises one or more of such sequence modifications.

### Expression Elements

Any polynucleotide encoding a variant Cas polypeptide or other CRISPR system component disclosed herein may be functionally linked to a heterologous expression element, to facilitate transcription or regulation in a host cell. Such expression elements include but are not limited to a promoter, leader, intron, and terminator. Expression elements may be “minimal”—meaning a shorter sequence derived from a native source, that still functions as an expression regulator or modifier. Alternatively, an expression element may be “optimized”—meaning that its polynucleotide sequence has been altered from its native state in order to function with a more desirable characteristic in a particular host cell (for example, but not limited to, a bacterial promoter may be “maize-optimized” to improve its expression in corn plants). Alternatively, an expression element may be “synthetic”—meaning that it is designed in silico and synthesized for use in a host cell. Synthetic expression elements may be entirely synthetic, or partially synthetic (comprising a fragment of a naturally occurring polynucleotide sequence).

It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters” if the promoters direct RNA synthesis preferably in certain tissues but also in other tissues at reduced levels.

A plant promoter includes a promoter capable of initiating transcription in a plant cell. For a review of plant promoters, see, Potenza et al. 2004, In vitro Cell Dev Biol 40:1-22; Porto et al. 2014, Molecular Biotechnology (2014), 56(1), 38-49.

Constitutive promoters include, for example, the core CaMV 35S promoter (Odell et al. (1985) Nature 313:810-2); rice actin (McElroy et al. (1990) Plant Cell 2:163-71); ubiquitin (Christensen et al. (1989) Plant Mol Biol 12:619-32; ALS promoter (U.S. Pat. No. 5,659,026) and the like.

Tissue-preferred promoters can be utilized to target enhanced expression within a particular plant tissue. Tissue-preferred promoters include, for example, WO2013103367 published 11 Jul. 2013, Kawamata et al. (1997) Plant Cell Physiol 38:792-803; Hansen et al. (1997)Mol Gen Genet 254:337-43; Russell et al. (1997) Transgenic Res 6:157-68; Rinehart et al. (1996) Plant Physiol 112:1331-41; Van Camp et al. (1996) Plant Physiol 112:525-35; Canevascini et al. (1996) Plant Physiol 112:513-524; Lam, (1994) Results Probl Cell Differ 20:181-96; and Guevara-Garcia et al. (1993) Plant J 4:495-505. Leaf-preferred promoters include, for example, Yamamoto et al. (1997) Plant J 12:255-65; Kwon et al. (1994) Plant Physiol 105:357-67; Yamamoto et al. (1994) Plant Cell Physiol 35:773-8; Gotor et al. (1993) Plant J3:509-18; Orozco et al. (1993) Plant Mol Biol 23:1129-38; Matsuoka et al. (1993) Proc. Natl. Acad. Sci. USA 90:9586-90; Simpson et al. (1958) EMBO J 4:2723-9; Timko et al. (1988) Nature 318:57-8. Root-preferred promoters include, for example, Hire et al. (1992) Plant Mol Biol 20:207-18 (soybean root-specific glutamine synthase gene); Miao et al. (1991) Plant Cell 3:11-22 (cytosolic glutamine synthase (GS)); Keller and Baumgartner, (1991) Plant Cell 3:1051-61 (root-specific control element in the GRP 1.8 gene of French bean); Sanger et al. (1990) Plant Mol Biol 14:433-43 (root-specific promoter of A. tumefaciens mannopine synthase (MAS)); Bogusz et al. (1990) Plant Cell 2:633-41 (root-specific promoters isolated from Parasponia andersonii and Trema tomentosa); Leach and Aoyagi, (1991) Plant Sci 79:69-76 (A. rhizogenes rolC and rolD root-inducing genes); Teeri et al. (1989) EMBOJ8:343-50 (Agrobacterium wound-induced TR1′ and TR2′ genes); VfENOD-GRP3 gene promoter (Kuster et al. (1995) Plant Mol Biol 29:759-72); and rolB promoter (Capana et al. (1994) Plant Mol Biol 25:681-91; phaseolin gene (Murai et al. (1983) Science 23:476-82; Sengopta-Gopalen et al. (1988) Proc. Natl. Acad. Sci. USA 82:3320-4). See also, U.S. Pat. Nos. 5,837,876; 5,750,386; 5,633,363; 5,459,252; 5,401,836; 5,110,732 and 5,023,179.

Seed-preferred promoters include both seed-specific promoters active during seed development, as well as seed-germinating promoters active during seed germination. See, Thompson et al. (1989) BioEssays 10:108. Seed-preferred promoters include, but are not limited to, Cim1 (cytokinin-induced message); cZ19B1 (maize 19 kDa zein); and milps (myo-inositol-1-phosphate synthase); and for example those disclosed in WO2000011177 published 2 Mar. 2000 and U.S. Pat. No. 6,225,529. For dicots, seed-preferred promoters include, but are not limited to, bean p-phaseolin, napin, 0-conglycinin, soybean lectin, cruciferin, and the like. For monocots, seed-preferred promoters include, but are not limited to, maize 15 kDa zein, 22 kDa zein, 27 kDa gamma zein, waxy, shrunken 1, shrunken 2, globulin 1, oleosin, and nuc1. See also, WO2000012733 published 9 Mar. 2000, where seed-preferred promoters from END1 and END2 genes are disclosed.

Chemical inducible (regulated) promoters can be used to modulate the expression of a gene in a prokaryotic and eukaryotic cell or organism through the application of an exogenous chemical regulator. The promoter may be a chemical-inducible promoter, where application of the chemical induces gene expression, or a chemical-repressible promoter, where application of the chemical represses gene expression. Chemical-inducible promoters include, but are not limited to, the maize In2-2 promoter, activated by benzene sulfonamide herbicide safeners (De Veylder et al. (1997) Plant Cell Physiol 38:568-77), the maize GST promoter (GST-II-27, WO1993001294 published 21 Jan. 1993), activated by hydrophobic electrophilic compounds used as pre-emergent herbicides, and the tobacco PR-1a promoter (Ono et al. (2004) Biosci Biotechnol Biochem 68:803-7) activated by salicylic acid. Other chemical-regulated promoters include steroid-responsive promoters (see, for example, the glucocorticoid-inducible promoter (Schena et al. (1991) Proc. Natl. Acad. Sci. USA 88:10421-5; McNellis et al. (1998) Plant J 14:247-257); tetracycline-inducible and tetracycline-repressible promoters (Gatz et al. (1991) Mol Gen Genet 227:229-37; U.S. Pat. Nos. 5,814,618 and 5,789,156).

Pathogen inducible promoters induced following infection by a pathogen include, but are not limited to those regulating expression of PR proteins, SAR proteins, beta-1,3-glucanase, chitinase, etc.

A stress-inducible promoter includes the RD29A promoter (Kasuga et al. (1999) Nature Biotechnol. 17:287-91). One of ordinary skill in the art is familiar with protocols for simulating stress conditions such as drought, osmotic stress, salt stress and temperature stress and for evaluating stress tolerance of plants that have been subjected to simulated or naturally occurring stress conditions.

Another example of an inducible promoter useful in plant cells, is the ZmCAS1 promoter, described in US20130312137 published 21 Nov. 2013.

New promoters of various types useful in plant cells are constantly being discovered; numerous examples may be found in the compilation by Okamuro and Goldberg, (1989) In The Biochemistry of Plants, Vol. 115, Stumpf and Conn, eds (New York, NY Academic Press), pp. 1-82.

Modification of Genomes with Novel CRISPR-Cas System Components

As described herein, a guided Cas endonuclease can recognize, bind to a DNA target sequence and introduce a single strand (nick) or double-strand break. Once a single or double-strand break is induced in the DNA, the cell's DNA repair mechanism is activated to repair the break. Error-prone DNA repair mechanisms can produce mutations at double-strand break sites. The most common repair mechanism to bring the broken ends together is the nonhomologous end-joining (NHEJ) pathway (Bleuyard et al. (2006) DNA Repair 5:1-12). The structural integrity of chromosomes is typically preserved by the repair, but deletions, insertions, or other rearrangements (such as chromosomal translocations) are possible (Siebert and Puchta, 2002, Plant Cell 14:1121-31; Pacher et al. 2007, Genetics 175:21-9).

DNA double-strand breaks appear to be an effective factor to stimulate homologous recombination pathways (Puchta et al. (1995) Plant Mol Biol 28:281-92; Tzfira and White, (2005) Trends Biotechnol 23:567-9; Puchta, (2005) J Exp Bot 56:1-14). Using DNA-breaking agents, a two- to nine-fold increase of homologous recombination was observed between artificially constructed homologous DNA repeats in plants (Puchta et al. (1995) Plant Mol Biol 28:281-92). In maize protoplasts, experiments with linear DNA molecules demonstrated enhanced homologous recombination between plasmids (Lyznik et al. (1991) Mol Gen Genet 230:209-18).

Homology-directed repair (HDR) is a mechanism in cells to repair double-stranded and single stranded DNA breaks. Homology-directed repair includes homologous recombination (HR) and single-strand annealing (SSA) (Lieber. 2010 Annu. Rev. Biochem. 79:181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks (Davis and Maizels. PNAS (0027-8424), 111 (10), p. E924-E932).

Alteration of the genome of a prokaryotic and eukaryotic cell or organism cell, for example, through homologous recombination (HR), is a powerful tool for genetic engineering. Homologous recombination has been demonstrated in plants (Halfter et al. (1992) Mol Gen Genet 231:186-93) and insects (Dray and Gloor, 1997, Genetics 147:689-99). Homologous recombination has also been accomplished in other organisms. For example, at least 150-200 bp of homology was required for homologous recombination in the parasitic protozoan Leishmania (Papadopoulou and Dumas, (1997) Nucleic Acids Res 25:4278-86). In the filamentous fungus Aspergillus nidulans, gene replacement has been accomplished with as little as 50 bp flanking homology (Chaveroche et al. (2000) Nucleic Acids Res 28:e97). Targeted gene replacement has also been demonstrated in the ciliate Tetrahymena thermophila (Gaertig et al. (1994) Nucleic Acids Res 22:5391-8). In mammals, homologous recombination has been most successful in the mouse using pluripotent embryonic stem cell lines (ES) that can be grown in culture, transformed, selected and introduced into a mouse embryo (Watson et al. 1992, Recombinant DNA, 2nd Ed., Scientific American Books distributed by WH Freeman & Co.).

### Gene Targeting

The guide polynucleotide/Cas systems described herein can be used for gene targeting.

In general, DNA targeting can be performed by cleaving one or both strands at a specific polynucleotide sequence in a cell with any of the engineered Cas polypeptides disclosed herein, which is associated with a suitable polynucleotide component. Once a single or double-strand break is induced in the DNA, the cell's DNA repair mechanism is activated to repair the break via nonhomologous end-joining (NHEJ) or Homology-Directed Repair (HDR) processes which can lead to modifications at the target site.

The length of the DNA sequence at the target site can vary, and includes, for example, target sites that are at least 12, 13, 14, 15, 16, 17, 18, 19, 20, 21, 22, 23, 24, 25, 26, 27, 28, 29, 30, or more than 30 nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by a Cas endonuclease.

Assays to measure the single or double-strand break of a target site by an endonuclease are known in the art and generally measure the overall activity and specificity of the agent on DNA substrates comprising recognition sites.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide a guide polynucleotide/Cas endonuclease complex to a unique DNA target site.

### Gene Editing

The process for editing a genomic sequence combining DSB and modification templates generally comprises: introducing into a host cell a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB. Genome editing using DSB-inducing agents, such as Cas-gRNA complexes, has been described, for example in US20150082478 published on 19 Mar. 2015, WO2015026886 published on 26 Feb. 2015, WO2016007347 published 14 Jan. 2016, and WO/2016/025131 published on 18 Feb. 2016.

Some uses for guide RNA/Cas endonuclease systems have been described (see for example: US20150082478 A1 published 19 Mar. 2015, WO2015026886 published 26 Feb. 2015, and US20150059010 published 26 Feb. 2015) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Proteins may be altered in various ways including amino acid substitutions, deletions, truncations, and insertions. Methods for such manipulations are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al. (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff et al. (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not expected to produce radical changes in the characteristics of the protein, and the effect of any substitution, deletion, insertion, or combination thereof can be evaluated by routine screening assays. Assays for double-strand-break-inducing activity are known and generally measure the overall activity and specificity of the agent on DNA substrates comprising target sites.

Described herein are methods for genome editing with a Cas endonuclease and complexes with a Cas endonuclease and a guide polynucleotide. Following characterization of the guide RNA and PAM sequence, components of the endonuclease and associated CRISPR RNA (crRNA) may be utilized to modify chromosomal DNA in other organisms including plants. To facilitate optimal expression and nuclear localization (for eukaryotic cells), the genes comprising the complex may be optimized as described in WO2016186953 published 24 Nov. 2016, and then delivered into cells as DNA expression cassettes by methods known in the art. The components necessary to comprise an active complex may also be delivered as RNA with or without modifications that protect the RNA from degradation or as mRNA capped or uncapped (Zhang, Y. et al. 2016, Nat. Commun. 7:12617) or Cas protein guide polynucleotide complexes (WO2017070032 published 27 Apr. 2017), or any combination thereof. Additionally, a part or part(s) of the complex and crRNA may be expressed from a DNA construct while other components are delivered as RNA with or without modifications that protect the RNA from degradation or as mRNA capped or uncapped (Zhang et al. 2016 Nat. Commun. 7:12617) or Cas protein guide polynucleotide complexes (WO2017070032 published 27 Apr. 2017) or any combination thereof. To produce crRNAs in-vivo, tRNA derived elements may also be used to recruit endogenous RNAses to cleave crRNA transcripts into mature forms capable of guiding the complex to its DNA target site, as described, for example, in WO2017105991 published 22 Jun. 2017. Nickase complexes may be utilized separately or concertedly to generate a single or multiple DNA nicks on one or both DNA strands. Furthermore, the cleavage activity of the Cas endonuclease may be deactivated by altering key catalytic residues in its cleavage domain (Sinkunas, T. et al. 2013, EMBO J. 32:385-394) resulting in a RNA guided helicase that may be used to enhance homology directed repair, induce transcriptional activation, or remodel local DNA structures. Moreover, the activity of the Cas cleavage and helicase domains may both be knocked-out and used in combination with other DNA cutting, DNA nicking, DNA binding, transcriptional activation, transcriptional repression, DNA remodeling, DNA deamination, DNA unwinding, DNA recombination enhancing, DNA integration, DNA inversion, and DNA repair agents.

The transcriptional direction of the tracrRNA for the CRISPR-Cas system (if present) and other components of the CRISPR-Cas system (such as variable targeting domain, crRNA repeat, loop, anti-repeat) can be deduced as described in WO2016186946 published 24 Nov. 2016, and WO2016186953 published 24 Nov. 2016.

As described herein, once the appropriate guide RNA requirement is established, the PAM preferences for each new system disclosed herein may be examined. If the cleavage complex results in degradation of the randomized PAM library, the complex can be converted into a nickase by disabling the ATPase dependent helicase activity either through mutagenesis of critical residues or by assembling the reaction in the absence of ATP as described previously (Sinkunas, T. et al. 2013, EMBO J. 32:385-394). Two regions of PAM randomization separated by two protospacer targets may be utilized to generate a double-stranded DNA break which may be captured and sequenced to examine the PAM sequences that support cleavage by the respective complex.

In one embodiment, the invention describes a method for modifying a target site in the genome of a cell, the method comprising introducing into a cell at least one PGEN described herein, and identifying at least one cell that has a modification at said target, wherein the modification at said target site is selected from the group consisting of (i) a replacement of at least one nucleotide, (ii) a deletion of at least one nucleotide, (iii) an insertion of at least one nucleotide, the chemical alteration of at least one nucleotide, and (v) any combination of (i)-(iv).

The nucleotide to be edited can be located within or outside a target site recognized and cleaved by a Cas endonuclease. In one embodiment, the at least one nucleotide modification is not a modification at a target site recognized and cleaved by a Cas endonuclease. In another embodiment, there are at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the genomic target site.

A knock-out may be produced by an indel (insertion or deletion of nucleotide bases in a target DNA sequence through NHEJ), or by specific removal of sequence that reduces or completely destroys the function of sequence at or near the targeting site.

A guide polynucleotide/Cas endonuclease induced targeted mutation can occur in a nucleotide sequence that is located within or outside a genomic target site that is recognized and cleaved by the Cas endonuclease.

The method for editing a nucleotide sequence in the genome of a cell can be a method without the use of an exogenous selectable marker by restoring function to a non-functional gene product.

In one embodiment, the invention describes a method for modifying a target site in the genome of a cell, the method comprising introducing into a cell at least one PGEN described herein and at least one donor DNA, wherein said donor DNA comprises a polynucleotide of interest, and optionally, further comprising identifying at least one cell that said polynucleotide of interest integrated in or near said target site.

In one aspect, the methods disclosed herein may employ homologous recombination (HR) to provide integration of the polynucleotide of interest at the target site.

Various methods and compositions can be employed to produce a cell or organism having a polynucleotide of interest inserted in a target site via activity of a CRISPR-Cas system component described herein. In one method described herein, a polynucleotide of interest is introduced into the organism cell via a donor DNA construct. As used herein, “donor DNA” is a DNA construct that comprises a polynucleotide of interest to be inserted into the target site of a Cas endonuclease. The donor DNA construct further comprises a first and a second region of homology that flank the polynucleotide of interest. The first and second regions of homology of the donor DNA share homology to a first and a second genomic region, respectively, present in or flanking the target site of the cell or organism genome.

The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al. 2013, Nature Methods Vol. 10:957-963).

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al. (1989) Molecular Cloning: A Laboratory Manual, (Cold Spring Harbor Laboratory Press, NY); Current Protocols in Molecular Biology, Ausubel et al. Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

Episomal DNA molecules can also be ligated into the double-strand break, for example, integration of T-DNAs into chromosomal double-strand breaks (Chilton and Que, (2003) Plant Physiol 133:956-65; Salomon and Puchta, (1998) EMBO J. 17:6086-95). Once the sequence around the double-strand breaks is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al. (2004) Plant Cell 16:342-52). Ectopic and/or epigenic DNA sequences may also serve as a DNA repair template for homologous recombination (Puchta, (1999) Genetics 152:1173-81).

In one embodiment, the disclosure comprises a method for editing a nucleotide sequence in the genome of a cell, the method comprising introducing into at least one PGEN described herein, and a polynucleotide modification template, wherein said polynucleotide modification template comprises at least one nucleotide modification of said nucleotide sequence, and optionally further comprising selecting at least one cell that comprises the edited nucleotide sequence.

The guide polynucleotide/Cas endonuclease system can be used in combination with at least one polynucleotide modification template to allow for editing (modification) of a genomic nucleotide sequence of interest. (See also US20150082478, published 19 Mar. 2015 and WO2015026886 published 26 Feb. 2015).

Polynucleotides of interest and/or traits can be stacked together in a complex trait locus as described in WO2012129373 published 27 Sep. 2012, and in WO2013112686, published 1 Aug. 2013. The guide polynucleotide/Cas9 endonuclease system described herein provides for an efficient system to generate double-strand breaks and allows for traits to be stacked in a complex trait locus.

A guide polynucleotide/Cas system as described herein, mediating gene targeting, can be used in methods for directing heterologous gene insertion and/or for producing complex trait loci comprising multiple heterologous genes in a fashion similar as disclosed in WO2012129373 published 27 Sep. 2012, where instead of using a double-strand break inducing agent to introduce a gene of interest, a guide polynucleotide/Cas system as disclosed herein is used. By inserting independent transgenes within 0.1, 0.2, 0.3, 0.4, 0.5, 1.0, 2, or even 5 centimorgans (cM) from each other, the transgenes can be bred as a single genetic locus (see, for example, US20130263324 published 3 Oct. 2013 or WO2012129373 published 14 Mar. 2013). After selecting a plant comprising a transgene, plants comprising (at least) one transgenes can be crossed to form an F1 that comprises both transgenes. In progeny from these F1 (F2 or BC1) 1/500 progeny would have the two different transgenes recombined onto the same chromosome. The complex locus can then be bred as single genetic locus with both transgene traits. This process can be repeated to stack as many traits as desired.

Further uses for guide RNA/Cas endonuclease systems have been described (See for example: US20150082478 published 19 Mar. 2015, WO2015026886 published 26 Feb. 2015, US20150059010 published 26 Feb. 2015, WO2016007347 published 14 Jan. 2016, and PCT application WO2016025131 published 18 Feb. 2016) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Resulting characteristics from the gene editing compositions and methods described herein may be evaluated. Chromosomal intervals that correlate with a phenotype or trait of interest can be identified. A variety of methods well known in the art are available for identifying chromosomal intervals. The boundaries of such chromosomal intervals are drawn to encompass markers that will be linked to the gene controlling the trait of interest. In other words, the chromosomal interval is drawn such that any marker that lies within that interval (including the terminal markers that define the boundaries of the interval) can be used as a marker for a particular trait. In one embodiment, the chromosomal interval comprises at least one QTL, and furthermore, may indeed comprise more than one QTL. Close proximity of multiple QTLs in the same interval may obfuscate the correlation of a particular marker with a particular QTL, as one marker may demonstrate linkage to more than one QTL. Conversely, e.g., if two markers in close proximity show co-segregation with the desired phenotypic trait, it is sometimes unclear if each of those markers identifies the same QTL or two different QTL. The term “quantitative trait locus” or “QTL” refers to a region of DNA that is associated with the differential expression of a quantitative phenotypic trait in at least one genetic background, e.g., in at least one breeding population. The region of the QTL encompasses or is closely linked to the gene or genes that affect the trait in question. An “allele of a QTL” can comprise multiple genes or other genetic factors within a contiguous genomic region or linkage group, such as a haplotype. An allele of a QTL can denote a haplotype within a specified window wherein said window is a contiguous genomic region that can be defined, and tracked, with a set of one or more polymorphic markers. A haplotype can be defined by the unique fingerprint of alleles at each marker within the specified window.

Introduction of CRISPR-Cas System Components into a Cell

The methods and compositions described herein do not depend on a particular method for introducing a sequence into an organism or cell, only that the polynucleotide or polypeptide gains access to the interior of at least one cell of the organism. Introducing includes reference to the incorporation of a nucleic acid into a eukaryotic or prokaryotic cell where the nucleic acid may be incorporated into the genome of the cell, and includes reference to the transient (direct) provision of a nucleic acid, protein or polynucleotide-protein complex (PGEN, RGEN) to the cell.

Methods for introducing polynucleotides or polypeptides or a polynucleotide-protein complex into cells or organisms are known in the art including, but not limited to, microinjection, electroporation, stable transformation methods, transient transformation methods, ballistic particle acceleration (particle bombardment), whiskers mediated transformation, Agrobacterium-mediated transformation, direct gene transfer, viral-mediated introduction, transfection, transduction, cell-penetrating peptides, mesoporous silica nanoparticle (MSN)-mediated direct protein delivery, topical applications, sexual crossing, sexual breeding, and any combination thereof.

For example, the guide polynucleotide (guide RNA, crNucleotide+tracrNucleotide, guide DNA and/or guide RNA-DNA molecule) can be introduced into a cell directly (transiently) as a single stranded or double stranded polynucleotide molecule. The guide RNA (or crRNA+tracrRNA) can also be introduced into a cell indirectly by introducing a recombinant DNA molecule comprising a heterologous nucleic acid fragment encoding the guide RNA (or crRNA+tracrRNA), operably linked to a specific promoter that is capable of transcribing the guide RNA (crRNA+tracrRNA molecules) in said cell. The specific promoter can be, but is not limited to, a RNA polymerase III promoter, which allow for transcription of RNA with precisely defined, unmodified, 5′- and 3′-ends (Ma et al. 2014, Mol. Ther. Nucleic Acids 3:e161; DiCarlo et al. 2013, Nucleic Acids Res. 41:4336-4343; WO2015026887, published 26 Feb. 2015). Any promoter capable of transcribing the guide RNA in a cell can be used and includes a heat shock/heat inducible promoter operably linked to a nucleotide sequence encoding the guide RNA.

Plant cells differ from animal cells (such as human cells), fungal cells (such as yeast cells) and protoplasts, including for example plant cells comprise a plant cell wall which may act as a barrier to the delivery of components.

Delivery of the Cas endonuclease, and/or the guide RNA, and/or a ribonucleoprotein complex, and/or a polynucleotide encoding any one or more of the preceding, into plant cells can be achieved through methods known in the art, for example but not limited to: Rhizobiales-mediated transformation (e.g., Agrobacterium, Ochrobactrum), particle mediated delivery (particle bombardment), polyethylene glycol (PEG)-mediated transfection (for example to protoplasts), electroporation, cell-penetrating peptides, or mesoporous silica nanoparticle (MSN)-mediated direct protein delivery.

The Cas endonuclease described herein, can be introduced into a cell by directly introducing the Cas polypeptide itself (referred to as direct delivery of Cas endonuclease), the mRNA encoding the Cas protein, and/or the guide polynucleotide/Cas endonuclease complex itself, using any method known in the art. The Cas endonuclease can also be introduced into a cell indirectly by introducing a recombinant DNA molecule that encodes the Cas endonuclease. The endonuclease can be introduced into a cell transiently or can be incorporated into the genome of the host cell using any method known in the art. Uptake of the endonuclease and/or the guided polynucleotide into the cell can be facilitated with a Cell Penetrating Peptide (CPP) as described in WO2016073433 published 12 May 2016. Any promoter capable of expressing the Cas endonuclease in a cell can be used and includes a heat shock/heat inducible promoter operably linked to a nucleotide sequence encoding the Cas endonuclease.

Direct delivery of a polynucleotide modification template into plant cells can be achieved through particle mediated delivery, and any other direct method of delivery, such as but not limiting to, polyethylene glycol (PEG)-mediated transfection to protoplasts, whiskers mediated transformation, electroporation, particle bombardment, cell-penetrating peptides, or mesoporous silica nanoparticle (MSN)-mediated direct protein delivery can be successfully used for delivering a polynucleotide modification template in eukaryotic cells, such as plant cells.

The donor DNA can be introduced by any means known in the art. The donor DNA may be provided by any transformation method known in the art including, for example, Agrobacterium-mediated transformation or biolistic particle bombardment. The donor DNA may be present transiently in the cell or it could be introduced via a viral replicon. In the presence of the Cas endonuclease and the target site, the donor DNA is inserted into the transformed plant's genome.

Direct delivery of any one of the guided Cas system components can be accompanied by direct delivery (co-delivery) of other mRNAs that can promote the enrichment and/or visualization of cells receiving the guide polynucleotide/Cas endonuclease complex components. For example, direct co-delivery of the guide polynucleotide/Cas endonuclease components (and/or guide polynucleotide/Cas endonuclease complex itself) together with mRNA encoding phenotypic markers (such as but not limiting to transcriptional activators such as CRC (Bruce et al. 2000 The Plant Cell 12:65-79) can enable the selection and enrichment of cells without the use of an exogenous selectable marker by restoring function to a non-functional gene product as described in WO2017070032 published 27 Apr. 2017.

Introducing a guide RNA/Cas endonuclease complex described herein, (representing the cleavage ready complex described herein) into a cell includes introducing the individual components of said complex either separately or combined into the cell, and either directly (direct delivery as RNA for the guide and protein for the Cas endonuclease and protein subunits, or functional fragments thereof) or via recombination constructs expressing the components (guide RNA, Cas endonuclease, protein subunits, or functional fragments thereof). Introducing a guide RNA/Cas endonuclease complex (RGEN) into a cell includes introducing the guide RNA/Cas endonuclease complex as a ribonucleotide-protein into the cell. The ribonucleotide-protein can be assembled prior to being introduced into the cell as described herein. The components comprising the guide RNA/Cas endonuclease ribonucleotide protein (at least one Cas endonuclease, at least one guide RNA, at least one protein subunit) can be assembled in vitro or assembled by any means known in the art prior to being introduced into a cell (targeted for genome modification as described herein).

Direct delivery of the RGEN ribonucleoprotein, allows for genome editing at a target site in the genome of a cell which can be followed by rapid degradation of the complex, and only a transient presence of the complex in the cell. This transient presence of the RGEN complex may lead to reduced off-target effects. In contrast, delivery of RGEN components (guide RNA, Cas9 endonuclease) via plasmid DNA sequences can result in constant expression of RGENs from these plasmids which can intensify off target effects (Cradick, T. J. et al. (2013) Nucleic Acids Res 41:9584-9592; Fu, Y et al. (2014) Nat. Biotechnol. 31:822-826).

Direct delivery can be achieved by combining any one component of the guide RNA/Cas endonuclease complex (RGEN), representing the cleavage ready complex described herein, (such as at least one guide RNA, at least one Cas protein, and optionally one additional protein), with a delivery matrix comprising a microparticle (such as but not limited to of a gold particle, tungsten particle, and silicon carbide whisker particle) (see also WO2017070032 published 27 Apr. 2017). The delivery matrix may comprise any one of the components, such as the Cas endonuclease, that is attached to a solid matrix (e.g., a particle for bombardment).

In one aspect the guide polynucleotide/Cas endonuclease complex, is a complex wherein the guide RNA and Cas endonuclease protein forming the guide RNA/Cas endonuclease complex are introduced into the cell as RNA and protein, respectively.

In one aspect the guide polynucleotide/Cas endonuclease complex, is a complex wherein the guide RNA and Cas endonuclease protein and the at least one protein subunit of a complex forming the guide RNA/Cas endonuclease complex are introduced into the cell as RNA and proteins, respectively.

In one aspect the guide polynucleotide/Cas endonuclease complex, is a complex wherein the guide RNA and Cas endonuclease protein and the at least one protein subunit of a complex forming the guide RNA/Cas endonuclease complex (cleavage ready complex) are preassembled in vitro and introduced into the cell as a ribonucleotide-protein complex.

Protocols for introducing polynucleotides, polypeptides or polynucleotide-protein complexes (PGEN, RGEN) into eukaryotic cells, such as plants or plant cells are known and include microinjection (Crossway et al. (1986) Biotechniques 4:320-34 and U.S. Pat. No. 6,300,543), meristem transformation (U.S. Pat. No. 5,736,369), electroporation (Riggs et al. (1986) Proc. Natl. Acad. Sci. USA 83:5602-6, Agrobacterium-mediated transformation (U.S. Pat. Nos. 5,563,055 and 5,981,840), whiskers mediated transformation (Ainley et al. 2013, Plant Biotechnology Journal 11:1126-1134; Shaheen A. and M. Arshad 2011 Properties and Applications of Silicon Carbide (2011), 345-358 Editor(s): Gerhardt, Rosario. Publisher: InTech, Rijeka, Croatia. CODEN:69PQBP; ISBN:978-953-307-201-2), direct gene transfer (Paszkowski et al. (1984) EMBOJ3:2717-22), and ballistic particle acceleration (U.S. Pat. Nos. 4,945,050; 5,879,918; 5,886,244; 5,932,782; Tomes et al. (1995) “Direct DNA Transfer into Intact Plant Cells via Microprojectile Bombardment” in Plant Cell, Tissue, and Organ Culture: Fundamental Methods, ed. Gamborg & Phillips (Springer-Verlag, Berlin); McCabe et al. (1988) Biotechnology 6:923-6; Weissinger et al. (1988) Ann Rev Genet 22:421-77; Sanford et al. (1987) Particulate Science and Technology 5:27-37 (onion); Christou et al. (1988) Plant Physiol 87:671-4 (soybean); Finer and McMullen, (1991) In vitro Cell Dev Biol 27P:175-82 (soybean); Singh et al. (1998) Theor Appl Genet 96:319-24 (soybean); Datta et al. (1990) Biotechnology 8:736-40 (rice); Klein et al. (1988) Proc. Natl. Acad. Sci. USA 85:4305-9 (maize); Klein et al. (1988) Biotechnology 6:559-63 (maize); U.S. Pat. Nos. 5,240,855; 5,322,783 and 5,324,646; Klein et al. (1988) Plant Physiol 91:440-4 (maize); Fromm et al. (1990) Biotechnology 8:833-9 (maize); Hooykaas-Van Slogteren et al. (1984) Nature 311:763-4; U.S. Pat. No. 5,736,369 (cereals); Bytebier et al. (1987) Proc. Natl. Acad. Sci. USA 84:5345-9 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Rep 9:415-8) and Kaeppler et al. (1992) Theor Appl Genet 84:560-6 (whisker-mediated transformation); D'Halluin et al. (1992) Plant Cell 4:1495-505 (electroporation); Li et al. (1993) Plant Cell Rep 12:250-5; Christou and Ford (1995) Annals Botany 75:407-13 (rice) and Osjoda et al. (1996) Nat Biotechnol 14:745-50 (maize via Agrobacterium tumefaciens).

Alternatively, polynucleotides may be introduced into plant or plant cells by contacting cells or organisms with a virus or viral nucleic acids. Generally, such methods involve incorporating a polynucleotide within a viral DNA or RNA molecule. In some examples a polypeptide of interest may be initially synthesized as part of a viral polyprotein, which is later processed by proteolysis in vivo or in vitro to produce the desired recombinant protein. Methods for introducing polynucleotides into plants and expressing a protein encoded therein, involving viral DNA or RNA molecules, are known, see, for example, U.S. Pat. Nos. 5,889,191, 5,889,190, 5,866,785, 5,589,367 and 5,316,931.

For Zea mays, cellular transformation methods disclosed herein include, for example, Agrobacterium-mediated delivery, Ensifer-based delivery, nanoparticle-mediated delivery, and particle-mediated biolistic delivery, as well as approaches utilizing protoplasts (Sardesai and Subramanyam (2018) Agrobacterium Biology: From Basic Science to Biotechnology. Cham: Springer International Publishing, 463-488, Rathore et al. (2019) Transgenic Plants: Methods and Protocols. New York, NY: Springer New York, 37-48, Wang et al. (2019) Molecular Plant. 12, 1037-1040, Rhodes et al. (1988) Science. 240, 204-207 and Golovkin et al. (1993) Plant Science. 90, 41-52). In the present Example, Cas-alpha endonuclease and guide RNA plasmid expression cassettes were either co-delivered into 9-10 day old maize immature embryos using particle-mediated biolistic transformation (Svitashev et al. (2015) Plant Physiology. 169, 931-945 and Karvelis et al. (2015) Genome Biology. 16, 253) or co-delivered on a single transfer DNA (T-DNA) into maize immature embryos using Agrobacterium as described earlier (Lowe et al. (2018) In vitro cellular & developmental biology-Plant. 54, 240-252).

For Saccharomyces cerevisiae, several methods (lithium acetate, polyethylene glycol (PEG), heat shock, electroporation, biolistic, and others) can be used for transformation (Kawai et al. (2010) Bioengineered Bugs. 1:395-403).

The polynucleotide or recombinant DNA construct can be provided to or introduced into a prokaryotic and eukaryotic cell or organism using a variety of transient transformation methods. Such transient transformation methods include, but are not limited to, the introduction of the polynucleotide construct directly into the cell.

Nucleic acids and proteins can be provided to a cell by any method including methods using molecules to facilitate the uptake of anyone or all components of a guided Cas system (protein and/or nucleic acids), such as cell-penetrating peptides and nanocarriers. See also US20110035836 published 10 Feb. 2011, and EP2821486A1 published 7 Jan. 2015.

Other methods of introducing polynucleotides into a prokaryotic and eukaryotic cell or organism or plant part can be used, including plastid transformation methods, and the methods for introducing polynucleotides into tissues from seedlings or mature seeds.

Stable transformation is intended to mean that the nucleotide construct introduced into an organism integrates into a genome of the organism and is capable of being inherited by the progeny thereof. Transient transformation is intended to mean that a polynucleotide is introduced into the organism and does not integrate into a genome of the organism or a polypeptide is introduced into an organism. Transient transformation indicates that the introduced composition is only temporarily expressed or present in the organism.

A variety of methods are available to identify those cells having an altered genome at or near a target site without using a screenable marker phenotype. Such methods can be viewed as directly analyzing a target sequence to detect any change in the target sequence, including but not limited to PCR methods, sequencing methods, nuclease digestion, Southern blots, and any combination thereof.

### Cells and Plants

The presently disclosed polynucleotides and polypeptides can be introduced into a cell. Cells include, but are not limited to, human, non-human, animal, mammalian, bacterial, fungal, insect, yeast, non-conventional yeast, and plant cells as well as plants and seeds produced by the methods described herein. Any plant can be used with the compositions and methods described herein, including monocot and dicot plants, and plant elements.

Examples of monocot plants that can be used include, but are not limited to, corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses.

Examples of dicot plants that can be used include, but are not limited to, soybean (Glycine max), Brassica species (for example but not limited to: oilseed rape or Canola) (Brassica napus, B. campestris, Brassica rapa, Brassica. juncea), alfalfa (Medicago sativa),), tobacco (Nicotiana tabacum), Arabidopsis (Arabidopsis thaliana), sunflower (Helianthus annuus), cotton (Gossypium arboreum, Gossypium barbadense), and peanut (Arachis hypogaea), tomato (Solanum lycopersicum), potato (Solanum tuberosum.

Additional plants that can be used include safflower (Carthamus tinctorius), sweet potato (Ipomoea batatus), cassava (Manihot esculenta), coffee (Coffea spp.), coconut (Cocos nucifera), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), vegetables, ornamentals, and conifers.

Vegetables that can be used include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be used include pines such as loblolly pine (Pinus taeda), slash pine (Pinus elliotii), ponderosa pine (Pinus ponderosa), lodgepole pine (Pinus contorta), and Monterey pine (Pinus radiata); Douglas fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow cedar (Chamaecyparis nootkatensis).

In certain embodiments of the disclosure, a fertile plant is a plant that produces viable male and female gametes and is self-fertile. Such a self-fertile plant can produce a progeny plant without the contribution from any other plant of a gamete and the genetic material comprised therein. Other embodiments of the disclosure can involve the use of a plant that is not self-fertile because the plant does not produce male gametes, or female gametes, or both, that are viable or otherwise capable of fertilization.

The present disclosure finds use in the breeding of plants comprising one or more introduced traits, or edited genomes.

A non-limiting example of how two traits can be stacked into the genome at a genetic distance of, for example, 5 cM from each other is described as follows: A first plant comprising a first transgenic target site integrated into a first DSB target site within the genomic window and not having the first genomic locus of interest is crossed to a second transgenic plant, comprising a genomic locus of interest at a different genomic insertion site within the genomic window and the second plant does not comprise the first transgenic target site. About 5% of the plant progeny from this cross will have both the first transgenic target site integrated into a first DSB target site and the first genomic locus of interest integrated at different genomic insertion sites within the genomic window. Progeny plants having both sites in the defined genomic window can be further crossed with a third transgenic plant comprising a second transgenic target site integrated into a second DSB target site and/or a second genomic locus of interest within the defined genomic window and lacking the first transgenic target site and the first genomic locus of interest. Progeny are then selected having the first transgenic target site, the first genomic locus of interest and the second genomic locus of interest integrated at different genomic insertion sites within the genomic window. Such methods can be used to produce a transgenic plant comprising a complex trait locus having at least 1, 2, 3, 4, 5, 6, 7, 8, 9, 10, 11, 12, 13, 14,15, 16, 17, 19, 19, 20, 21, 22, 23, 24, 25, 26, 27, 28, 29, 30, 31 or more transgenic target sites integrated into DSB target sites and/or genomic loci of interest integrated at different sites within the genomic window. In such a manner, various complex trait loci can be generated.

### Cells and Animals

The presently disclosed polynucleotides and polypeptides can be introduced into an animal cell. Animal cells can include, but are not limited to: an organism of a phylum including chordates, arthropods, mollusks, annelids, cnidarians, or echinoderms; or an organism of a class including mammals, insects, birds, amphibians, reptiles, or fishes. In some aspects, the animal is human, mouse, C. elegans, rat, fruit fly (Drosophila spp.), zebrafish, chicken, dog, cat, guinea pig, hamster, chicken, Japanese ricefish, sea lamprey, pufferfish, tree frog (e.g., Xenopus spp.), monkey, or chimpanzee. Particular cell types that are contemplated include haploid cells, diploid cells, reproductive cells, neurons, muscle cells, endocrine or exocrine cells, epithelial cells, muscle cells, tumor cells, embryonic cells, hematopoietic cells, bone cells, germ cells, somatic cells, stem cells, pluripotent stem cells, induced pluripotent stem cells, progenitor cells, meiotic cells, and mitotic cells. In some aspects, a plurality of cells from an organism may be used.

The novel engineered Cas polypeptides disclosed may be used to edit the genome of an animal cell in various ways. In one aspect, it may be desirable to delete one or more nucleotides. In another aspect, it may be desirable to insert one or more nucleotides. In one aspect, it may be desirable to replace one or more nucleotides. In another aspect, it may be desirable to modify one or more nucleotides via a covalent or non-covalent interaction with another atom or molecule.

Genome modification via a disclosed engineered Cas polypeptide may be used to effect a genotypic and/or phenotypic change on the target organism. Such a change is preferably related to an improved phenotype of interest or a physiologically important characteristic, the correction of an endogenous defect, or the expression of some type of expression marker. In some aspects, the phenotype of interest or physiologically important characteristic is related to the overall health, fitness, or fertility of the animal, the ecological fitness of the animal, or the relationship or interaction of the animal with other organisms in its environment. In some aspects, the phenotype of interest or physiologically-important characteristic is selected from the group consisting of: improved general health, disease reversal, disease modification, disease stabilization, disease prevention, treatment of parasitic infections, treatment of viral infections, treatment of retroviral infections, treatment of bacterial infections, treatment of neurological disorders (for example but not limited to: multiple sclerosis), correction of endogenous genetic defects (for example but not limited to: metabolic disorders, Achondroplasia, Alpha-1 Antitrypsin Deficiency, Antiphospholipid Syndrome, Autism, Autosomal Dominant Polycystic Kidney Disease, Barth syndrome, Breast cancer, Charcot-Marie-Tooth, Colon cancer, Cri du chat, Crohn's Disease, Cystic fibrosis, Dercum Disease, Down Syndrome, Duane Syndrome, Duchenne Muscular Dystrophy, Factor V Leiden Thrombophilia, Familial Hypercholesterolemia, Familial Mediterranean Fever, Fragile X Syndrome, Gaucher Disease, Hemochromatosis, Hemophilia, Holoprosencephaly, Huntington's disease, Klinefelter syndrome, Marfan syndrome, Myotonic Dystrophy, Neurofibromatosis, Noonan Syndrome, Osteogenesis Imperfecta, Parkinson's disease, Phenylketonuria, Poland Anomaly, Porphyria, Progeria, Prostate Cancer, Retinitis Pigmentosa, Severe Combined Immunodeficiency (SCID), Sickle cell disease, Skin Cancer, Spinal Muscular Atrophy, Tay-Sachs, Thalassemia, Trimethylaminuria, Turner Syndrome, Velocardiofacial Syndrome, WAGR Syndrome, and Wilson Disease), treatment of innate immune disorders (for example but not limited to: immunoglobulin subclass deficiencies), treatment of acquired immune disorders (for example but not limited to: AIDS and other HIV-related disorders), treatment of cancer, as well as treatment of diseases, including rare or “orphan” conditions, that have eluded effective treatment options with other methods.

Cells that have been genetically modified using the compositions or methods disclosed herein may be transplanted to a subject for purposes such as gene therapy, e.g. to treat a disease, or as an antiviral, antipathogenic, or anticancer therapeutic, for the production of genetically modified organisms in agriculture, or for biological research.

### In Vitro Polynucleotide Detection, Binding, and Modification

The compositions disclosed herein may further be used as compositions for use in in vitro methods, in some aspects with isolated polynucleotide sequence(s). Said isolated polynucleotide sequence(s) may comprise one or more target sequence(s) for modification. In some aspects, said isolated polynucleotide sequence(s) may be genomic DNA, a PCR product, or a synthesized oligonucleotide.

### Compositions

Modification of a target sequence may be in the form of a nucleotide insertion, a nucleotide deletion, a nucleotide substitution, the addition of an atom molecule to an existing nucleotide, a nucleotide modification, or the binding of a heterologous polynucleotide or polypeptide to said target sequence. The insertion of one or more nucleotides may be accomplished by the inclusion of a donor polynucleotide in the reaction mixture: said donor polynucleotide is inserted into a double-strand break created by an engineered Cas endonuclease disclosed herein. The insertion may be via non-homologous end joining or via homologous recombination.

In one aspect, the sequence of the target polynucleotide is known prior to modification, and compared to the sequence(s) of polynucleotide(s) that result from treatment with the engineered Cas endonuclease. In one aspect, the sequence of the target polynucleotide is not known prior to modification, and the treatment with the engineered Cas endonuclease is used as part of a method to determine the sequence of said target polynucleotide.

Polynucleotide modification with an engineered Cas polypeptide may be accomplished by usage of a full-length polypeptide identified from a Cas locus, or from a fragment, modification, or variant of a polypeptide identified from a Cas locus. In some aspects, said Cas polypeptide variant is a polypeptide sharing at least 80% identity with any of SEQ ID NOs:15-47. In some aspects, said Cas polypeptide variant is a functional variant of any of any of SEQ ID NOs:15-47. In some aspects, said Cas polypeptide variant is a functional fragment of any of any of SEQ ID NOs:15-47. In some aspects, said Cas polypeptide is a Cas endonuclease polypeptide that recognizes a PAM sequence of N(T>W>C)TTC. In some aspects, the Cas polypeptide variant is provided by way of a polynucleotide encoding said Cas polypeptide.

In some aspects, the engineered Cas polypeptide may be selected from the group consisting of: an engineered or modified wild type Cas endonuclease ortholog, a functional Cas endonuclease ortholog variant, a functional engineered Cas polypeptide fragment, a fusion protein comprising an active or deactivated engineered Cas polypeptide variant, an engineered Cas polypeptide further comprising one or more nuclear localization sequences (NLS) on the C-terminus or on the N-terminus or on both the N- and C-termini, a biotinylated engineered Cas polypeptide, an engineered Cas endonuclease nickase, an engineered Cas polypeptide ortholog endonuclease, an engineered Cas polypeptide further comprising a Histidine tag, and a mixture of any two or more of the preceding.

In some aspects, the engineered Cas polypeptide is a fusion protein further comprising a nuclease domain, a transcriptional activator domain, a transcriptional repressor domain, an epigenetic modification domain, a cleavage domain, a nuclear localization signal, a cell-penetrating domain, a translocation domain, a marker, or a transgene that is heterologous to the target polynucleotide sequence or to the cell from which said target polynucleotide sequence is obtained or derived.

In some aspects, a plurality of engineered Cas polypeptides may be desired. In some aspects, said plurality may comprise engineered Cas polypeptides derived from different source organisms or from different loci within the same organism. In some aspects, said plurality may comprise engineered Cas polypeptides with different binding specificities to the target polynucleotide. In some aspects, said plurality may comprise engineered Cas endonucleases with different cleavage efficiencies. In some aspects, said plurality may comprise engineered Cas polypeptides with different PAM specificities. In some aspects, said plurality may comprise engineered Cas polypeptide of different molecular compositions, i.e., a polynucleotide encoding an engineered Cas polypeptide and a polypeptide that is an engineered Cas polypeptide.

The engineered Cas polypeptide disclosed herein may be used with a guide polynucleotide, which may be provided as a single guide RNA (sgRNA), a chimeric molecule comprising a tracrRNA, a chimeric molecule comprising a crRNA, a chimeric RNA-DNA molecule, a DNA molecule, or a polynucleotide comprising one or more chemically modified nucleotides.

The storage conditions of the engineered Cas polypeptide and/or the guide polynucleotide include parameters for temperature, state of matter, and time. In some aspects, the Cas polypeptide and/or the guide polynucleotide is stored at about −80 degrees Celsius, at about −20 degrees Celsius, at about 4 degrees Celsius, at about 20-25 degrees Celsius, or at about 37 degrees Celsius. In some aspects, the Cas polypeptide and/or the guide polynucleotide is stored as a liquid, a frozen liquid, or as a lyophilized powder. In some aspects, the Cas polypeptide and/or the guide polynucleotide is stable for at least one day, at least one week, at least one month, at least one year, or even greater than one year.

Any or all of the possible polynucleotide components of the reaction (e.g., guide polynucleotide, donor polynucleotide, optionally a polynucleotide encoding the Cas polypeptide) may be provided as part of a vector, a construct, a linearized or circularized plasmid, or as part of a chimeric molecule. Each component may be provided to the reaction mixture separately or together. In some aspects, one or more of the polynucleotide components are operably linked to a heterologous noncoding regulatory element that regulates its expression.

The method for modification of a target polynucleotide comprises combining the minimal elements into a reaction mixture comprising: an engineered Cas polypeptide (or variant, fragment, or other related molecule as described above), a guide polynucleotide comprising a sequence that is substantially complementary to, or selectively hybridizes to, the target polynucleotide sequence of the target polynucleotide, and a target polynucleotide for modification. In some aspects, the engineered Cas polypeptide is provided as a polypeptide. In some aspects, the engineered Cas polypeptide is provided as a Cas polypeptide polynucleotide. In some aspects, the guide polynucleotide is provided as an RNA molecule, a DNA molecule, an RNA:DNA hybrid, or a polynucleotide molecule comprising a chemically modified nucleotide.

The storage buffer of any one of the components, or the reaction mixture, may be optimized for stability, efficacy, or other parameters. Additional components of the storage buffer or the reaction mixture may include a buffer composition, Tris, EDTA, dithiothreitol (DTT), phosphate-buffered saline (PBS), sodium chloride, magnesium chloride, HEPES, glycerol, BSA, a salt, an emulsifier, a detergent, a chelating agent, a redox reagent, an antibody, nuclease-free water, a proteinase, and/or a viscosity agent. In some aspects, the storage buffer or reaction mixture further comprises a buffer solution with at least one of the following components: HEPES, MgCl2, NaCl, EDTA, a proteinase, Proteinase K, glycerol, nuclease-free water.

Incubation conditions will vary according to desired outcome. The temperature is preferably at least 10 degrees Celsius, between 10 and 15, at least 15, between 15 and 17, at least 17, between 17 and 20, at least 20, between 20 and 22, at least 22, between 22 and 25, at least 25, between 25 and 27, at least 27, between 27 and 30, at least 30, between 30 and 32, at least 32, between 32 and 35, at least 35, at least 36, at least 37, at least 38, at least 39, at least 40, or even greater than 40 degrees Celsius. The time of incubation is at least 1 minute, at least 2 minutes, at least 3 minutes, at least 4 minutes, at least 5 minutes, at least 6 minutes, at least 7 minutes, at least 8 minutes, at least 9 minutes, at least 10 minutes, or even greater than 10 minutes.

The sequence(s) of the polynucleotide(s) in the reaction mixture prior to, during, or after incubation may be determined by any method known in the art. In one aspect, modification of a target polynucleotide may be ascertained by comparing the sequence(s) of the polynucleotide(s) purified from the reaction mixture to the sequence of the target polynucleotide prior to combining with the engineered Cas polypeptide disclosed herein.

Any one or more of the compositions disclosed herein, useful for in vitro or in vivo polynucleotide detection, binding, and/or modification, may be comprised within a kit. A kit comprises an engineered Cas polypeptide (e.g., engineered Cas endonuclease) disclosed herein or a polynucleotide encoding the engineered Cas polypeptide or endonuclease, optionally further comprising buffer components to enable efficient storage, and one or more additional compositions that enable the introduction of said engineered Cas polypeptide to a cell or system comprising a heterologous polynucleotide, wherein said engineered Cas polypeptide is capable of effecting a modification, addition, deletion, or substitution of at least one nucleotide of said heterologous polynucleotide. In an additional aspect, a engineered Cas polypeptide disclosed herein may be used for the enrichment of one or more polynucleotide target sequences from a mixed pool. In an additional aspect, an engineered Cas polypeptide disclosed herein may be immobilized on a matrix for use in in vitro target polynucleotide detection, binding, and/or modification.

An engineered Cas endonuclease may be attached, associated with, or affixed to a solid matrix for the purposes of storage, purification, and/or characterization. Examples of a solid matrix include, but are not limited to: a filter, a chromatography resin, an assay plate, a test tube, a cryogenic vial, etc. A Cas endonuclease may be substantially purified and stored in an appropriate buffer solution, or lyophilized.

### Methods of Detection

Methods of detecting the engineered Cas polypeptide:guide polynucleotide complex bound to the target polynucleotide may include any known in the art, including but not limited to microscopy, chromatographic separation, electrophoresis, immunoprecipitation, filtration, nanopore separation, microarrays, as well as those described below.

A DNA Electrophoretic Mobility Shift Assay (EMSA): studies proteins binding to known DNA oligonucleotide probes and assesses the specificity of the interaction. The technique is based on the principle that protein-DNA complexes migrate more slowly than free DNA molecules when subjected to polyacrylamide or agarose gel electrophoresis. Because the rate of DNA migration is retarded upon protein binding, the assay is also called a gel retardation assay. Adding a protein-specific antibody to the binding components creates an even larger complex (antibody-protein-DNA) which migrates even slower during electrophoresis, this is known as a supershift and can be used to confirm protein identities.

DNA Pull-down Assays use a DNA probe labelled with a high affinity tag, such as biotin, which allows the probe to be recovered or immobilized. A DNA probe can be complexed with a protein from a cell lysate in a reaction similar to that used in the EMSA and then used to purify the complex using agarose or magnetic beads. The proteins are then eluted from the DNA and detected by Western blot or identified by mass spectrometry. Alternatively, the protein may be labelled with an affinity tag or the DNA-protein complex may be isolated using an antibody against the protein of interest (similar to a supershift assay). In this case, the unknown DNA sequence bound by the protein is detected by Southern blotting or through PCR analysis.

Reporter assays provide a real-time in vivo read-out of translational activity for a promoter of interest. Reporter genes are fusions of a target promoter DNA sequence and a reporter gene DNA sequence which is customized by the researcher and the DNA sequence codes for a protein with detectable properties like firefly/Renilla luciferase or alkaline phosphatase. These genes produce enzymes only when the promoter of interest is activated. The enzyme, in turn, catalyses a substrate to produce either light or a color change that can be detected by spectroscopic instrumentation. The signal from the reporter gene is used as an indirect determinant for the translation of endogenous proteins driven from the same promoter.

Microplate Capture and Detection Assays use immobilized DNA probes to capture specific protein-DNA interactions and confirm protein identities and relative amounts with target specific antibodies. Typically, a DNA probe is immobilized on the surface of 96- or 384-well microplates coated with streptavidin. A cellular extract is prepared and added to allow the binding protein to bind to the oligonucleotide. The extract is then removed and each well is washed several times to remove non-specifically bound proteins. Finally, the protein is detected using a specific antibody labelled for detection. This method can be extremely sensitive, detecting less than 0.2 pg of the target protein per well. This method may also be utilized for oligonucleotides labelled with other tags, such as primary amines that can be immobilized on microplates coated with an amine-reactive surface chemistry.

DNA Footprinting is one of the most widely used methods for obtaining detailed information on the individual nucleotides in protein-DNA complexes, even inside living cells. In such an experiment, chemicals or enzymes are used to modify or digest the DNA molecules.
When sequence specific proteins bind to DNA, they can protect the binding sites from modification or digestion. This can subsequently be visualized by denaturing gel electrophoresis, where unprotected DNA is cleaved more or less at random. Therefore it appears as a ‘ladder’ of bands and the sites protected by proteins have no corresponding bands and look like footprints in the pattern of bands. The footprints there by identify specific nucleosides at the protein-DNA binding sites.

Microscopic techniques include optical, fluorescence, electron, and atomic force microscopy (AFM).

Chromatin immunoprecipitation analysis (ChTP) causes proteins to bind covalently to their DNA targets, after which they are unlinked and characterized separately.

Systematic Evolution of Ligands by EXponential enrichment (SELEX) exposes target proteins to a random library of oligonucleotides. Those genes that bind are separated and amplified by PCR.

While the invention has been particularly shown and described with reference to a preferred embodiment and various alternate embodiments, it will be understood by persons skilled in the relevant art that various changes in form and details can be made therein without departing from the spirit and scope of the invention. For instance, while the particular examples below may illustrate the methods and embodiments described herein using a specific target site or target organism, the principles in these examples may be applied to any target site or target organism. Therefore, it will be appreciated that the scope of this invention is encompassed by the embodiments of the inventions recited herein and in the specification rather than the specific examples that are exemplified below. All cited patents, applications, and publications referred to in this application are herein incorporated by reference in their entirety, for all purposes, to the same extent as if each were individually and specifically incorporated by reference, except for any definitions, subject matter disclaimers or disavowals, and except to the extent that the incorporated material is inconsistent with the express disclosure herein, in which case the language in this disclosure controls.

### EXAMPLES

The following are Examples disclose specific embodiments of some aspects of the invention. The Examples are offered for illustrative purposes only, and are not intended to limit the scope of the invention in any way. Efforts have been made to ensure accuracy with respect to numbers used (e.g., amounts, temperatures, etc.), but some experimental error and deviation should, of course, be allowed for.

### Example 1: Cas-Alpha Nuclease and Guide RNA Expression Cassettes

In this Example, methods for generating Cas-alpha endonuclease and guide RNA expression cassettes for use in Saccharomyces cerevisiae, Zea mays, and Homo sapiens cells are described.

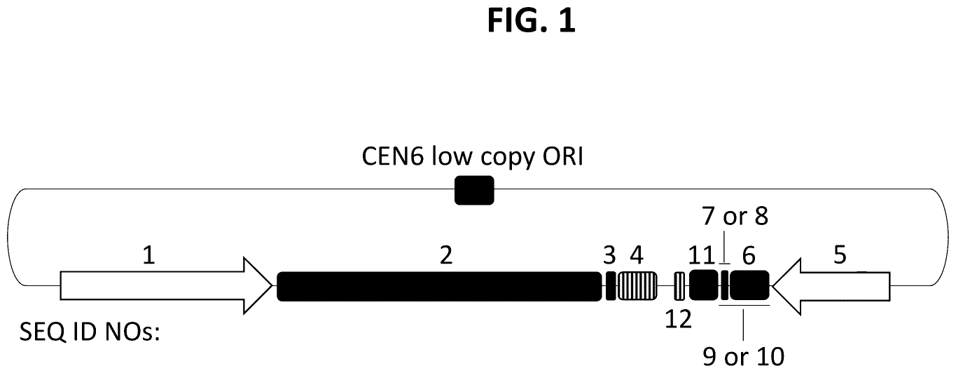

For S. cerevisiae, the gene encoding the Cas-alpha endonuclease was yeast codon optimized. To facilitate nuclear localization of the optimized Cas-alpha endonuclease protein, a nucleotide sequence encoding the Simian virus 40 (SV40) monopartite nuclear localization signal (NLS) (PKKKRKV (SEQ ID NO:13)) was added to either the 5′ and/or 3′ ends. Nucleotide sequences of the optimized Cas-alpha endonuclease gene and NLS variants were then synthesized and operably cloned into a low copy yeast plasmid DNA containing a CEN6 origin of replication (ORI) between a ROX3 promoter and CYC1 terminator (GenScript). An example of a yeast optimized Cas-alpha nuclease expression cassette and references to the sequences described herein can be found in FIG. 1.

The Cas-alpha endonuclease is directed by small RNAs (referred to herein as guide RNAs) to cleave double-stranded DNA in the presence of a 5′ protospacer adjacent motif (PAM) (Bigelyte et al. (2021), Nature Communications. 12:6191, Karvelis et al. (2020), Nucleic Acids Research. 48:5016-5023, U.S. Patent Application Publication No. US20200190494A1, and International Patent Application Publication WO 2022/082179). These guide RNAs comprise a sequence that aids recognition by Cas-alpha (referred to as Cas-alpha recognition domain) and a sequence that serves to direct Cas-alpha cleavage by base pairing with one strand of the DNA target site (Cas-alpha variable targeting domain). To transcribe small RNAs necessary for directing Cas-alpha endonuclease cleavage activity in S. cerevisiae cells, DNA sequences encoding the Hepatitis Delta Virus ribozyme was first appended to the 3′ end of a DNA sequence encoding a Cas-alpha single guide RNA (sgRNA) with a variable targeting domain capable of targeting the yeast ADE2 or CAN1 genes. Next, the SNR52 promoter and SUP4 terminator were operably linked to the ends of the ribozyme and Cas-alpha encoding sgRNA incorporating a G bp onto the 3′ end of the SNR52 to promote transcription of the Cas-alpha sgRNA. DNA fragments were then synthesized and cloned into the S. cerevisiae CEN6 vector containing the cas-alpha gene (GenScript). Schematic diagram of a yeast optimized Cas-alpha guide RNA expression cassette and references to the sequences described herein is shown in FIG. 1.

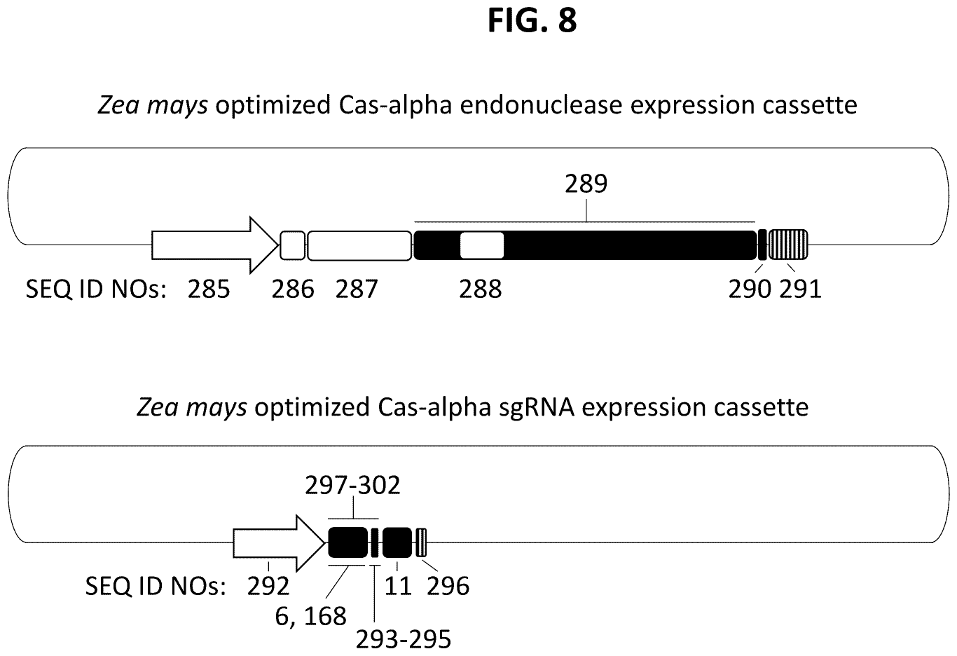

Zea mays Cas-alpha nuclease expression cassettes were built generally as described above, except that the sequence encoding the nuclease was codon optimized using Zea mays codon tables, GC content was adjusted using standard techniques, and repetitive sequences and gene destabilizing features such as miniature inverted-repeat transposable elements (MITEs) were removed. The resulting gene was then synthesized (GenScript) and cloned by restriction enzyme digestion and ligation into a Gateway compatible plasmid DNA containing a polymerase II promoter and terminator. For expression in maize, this includes a Zea mays Ubiquitin (UBI) promoter and terminator. To further enhance expression, a 5′ untranslated region (UTR) (for example but not limited to the maize UBI 5′ UTR) and additional introns (for example the UBI Zea mays intron 1 and potato ST-LS1 intron 2) are used although other introns would work. Schematic diagram of a maize optimized Cas-alpha endonuclease expression construct is shown in FIG. 8, upper diagram.

Cas-alpha sgRNA expression cassettes were built generally as described above, except that a maize U6 promoter and terminator were utilized and a variable targeting domain capable of directing the Cas-alpha endonuclease to the Zea mays MS26 (SEQ ID NO:303), CR36 (SEQ ID NO:304), or D8 (SEQ ID NO:305) target sites was incorporated. Schematic diagram of a maize optimized Cas-alpha sgRNA expression cassette is shown in FIG. 8, lower diagram.

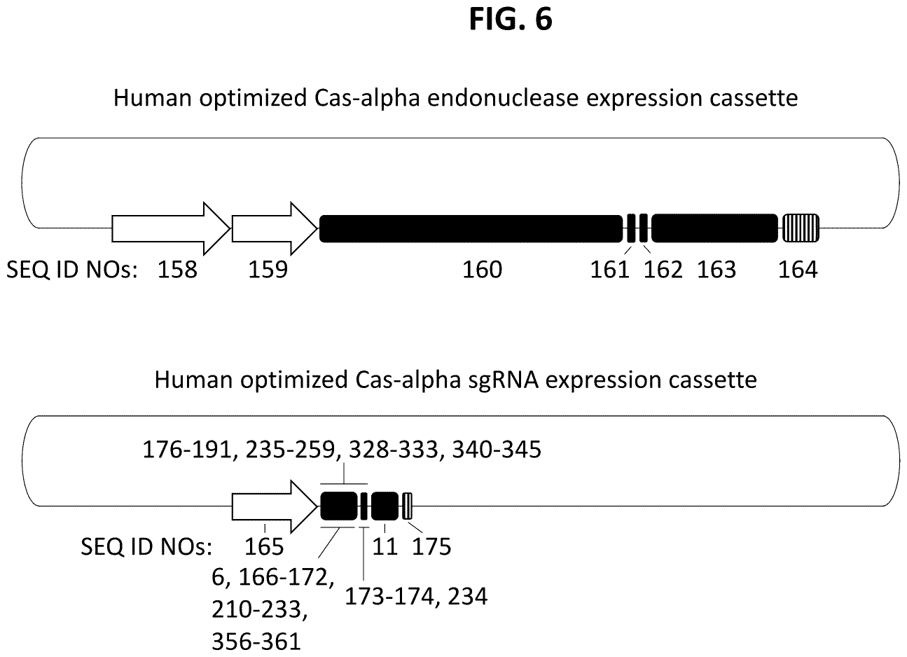

For tests in human cells, Cas-alpha endonuclease expression cassettes for SEQ ID NOs:14, 60, 157, and 362 were constructed, also generally as described above, except that the sequence encoding the nuclease was human codon-optimized, sequences encoding a ribosome skipping T2A peptide (Ahier et al. (2014) Genetics. 196:605-613) and green fluorescent protein (GFP) were fused in-frame to the 3′ end of the Cas-alpha endonuclease open-reading frame, a human betaherpesvirus promoter and enhancer were used to drive expression, and a sequence from the human gammaherpesvirus used to terminate transcription. Schematic diagram of a human-optimized Cas-alpha nuclease expression cassette is shown in FIG. 6, upper diagram.

Cas-alpha sgRNA expression cassettes were built next as described above, except that a human U6 promoter and terminator were utilized and a variable targeting domain capable of directing the Cas-alpha endonuclease to the human RUNX (SEQ ID NO:306), WTAP (SEQ ID NO:307), or DNMT (SEQ ID NO:308) target sites was incorporated. Schematic diagram of a human-optimized Cas-alpha sgRNA expression cassette is shown in FIG. 6, lower diagram.

### Example 2: Cas-Alpha Nuclease and Guide RNA Expression Cassette Transformation

In this Example, methods for transforming Cas-alpha endonuclease and guide RNA expression cassettes into Saccharomyces cerevisiae, Zea mays, and Homo sapiens cells are described.

For Saccharomyces cerevisiae, an approach similar to a lithium cation-based method using the Frozen-EZ yeast Transformation II kit (Zymo Research) was used. Per the manufacture's instruction, S. cerevisiae competent cells were produced by growing S. cerevisiae (BY4742 (Baker et al. (1998) Yeast. 14:115-132) (ATCC)) in yeast extract-peptone-dextrose (YPD) broth (Gibco) to mid-log phase corresponding to an OD 600 nm of 0.8-1.0. Next, the cells were pelleted by centrifugation (500×g for 4 minutes), media decanted, and the pellet gently washed with 10 ml of EZ 1 solution spinning down the cells again prior to removing the wash solution. Cells were then resuspended in 1 ml of EZ 2 solution and aliquoted and either stored at −70° C. or used in the next step. Transformation was performed next by adding 0.5-1 μg (in less than 5 μl) of the CEN6 yeast plasmid DNA containing the Cas-alpha nuclease and single guide RNA expression cassettes to 50 μl of competent cells. Optionally, double-stranded DNA repair template with homology flanking the expected Cas-alpha double-strand break site was also included (0.5 μl at 50 μM). After gently mixing in the DNA, 500 μl of EZ 3 solution was added. Next, cells were incubated at 30° C. for 60-90 min. flicking or vortexing the cells 3-4 times over the duration of the incubation. After transformation, cells were grown-out in YPD broth for ~3 hours, pelleted, washed once with 1 ml of sterile water, resuspended in 1 ml of sterile water, and then ~200 μl plated onto selective media (for example but not limited to 6.7 g/L yeast nitrogen base without amino acids (Becton Dickinson), 20 g/L glucose (Phytotechnology Labs), 1.92 g/L yeast histidine dropout media (MP Biomedicals) and 20 g/L Bacto Agar (Becton Dickinson)). To determine the culture conditions optimal for activity, cells were incubated at 30° C. until colonies formed, or at a range of temperatures (typically 25° C., 30° C., 37° C. and 45° C.) overnight and then placed back at 30° C. until colony growth was visible.

For Zea mays transformation, particle-mediated biolistic transformation, DNA expression cassettes were co-precipitated onto 0.6 μM (average size) gold particles utilizing TransIT-2020. DNA-coated gold particles were pelleted by centrifugation, washed with absolute ethanol and re-dispersed by sonication. Following sonication, 10 μl of the DNA-coated gold particles were loaded onto a macrocarrier and air dried. Biolistic transformation was performed using a PDS-1000/He Gun (Bio-Rad) with a 425 pound per square inch rupture disc. To promote cell division, BabyBoom (BBM) and Wuschel2 (WUS2) genes expressed from non-constitutive promoters, maize phospholipid transferase protein, and maize auxin-inducible, respectively, were also co-delivered alongside the Cas-alpha endonuclease and guide RNA expression cassettes (Lowe et al. (2018) In vitro cellular & developmental biology-Plant. 54:240-252). A visual marker DNA expression cassette encoding a fluorescent protein, for example, a yellow fluorescent protein, can also be co-delivered to aid in the selection of evenly transformed tissue. Moreover, a chemical selectable marker (for example but not limited to neomycin phosphotransferase II) can also be delivered with the aforementioned plasmids to select for transformed cells. To examine Cas-alpha nuclease-guide RNA activity, transformed tissue can be incubated at 28° C., standard for particle gun transformation, or at a range of temperatures lower or higher than 28° C.

For Agrobacterium delivery, the Cas-alpha nuclease and associated sgRNA expression cassettes were Gateway cloned into a T-DNA optionally already containing genes encoding the morphogenic transcription factors Bbm and Wus2 operably linked to Zm-PLTP and Axig1 promoters, respectively. The resulting T-DNA was then placed into LBA4404, a thymine auxotrophic strain of Agrobacterium tumefaciens, containing Vir9, a separate plasmid encoding the Bo542 virulence genes (US20170121722A1 and WO 2017/078836). The resulting Agrobacterium strain was then used to transform nine-to-ten-day old immature maize embryos (approximately 2 mm in size) by submersion in 700A liquid media containing the strain at an optical density (OD) of 0.4 to 2.0 at 550 nm for 5 min. and then removed from the media and placed on solid co-cultivation medium overnight at 21° C. in the dark. Following T-DNA delivery, embryos are transferred to resting media, 13266R, and grown in the dark at 28° C. for 5-7 days. Next, coleoptiles were removed, and embryos transferred to selection media for 11-16 days and kept in the dark at 28° C. They were then moved to maturation media and incubated for 14-25 days in the dark at 28° C. and then subject to light for 2-5 days. Next, they were moved to rooting media and incubated at 26-28° C. under light for 14-28 days refreshing media as needed. To examine Cas-alpha nuclease-guide RNA activity, transformed tissue was incubated at 28° C., standard for Agrobacterium transformation, or at a range of temperatures lower or higher than 28° C.

Human cell transformation was carried-out by culturing HEK293T cells (ATCC catalog umber CRL-3216) in Dulbecco's Modified Eagle Medium (DMEM) supplemented with 10% fetal bovine serum (FBS) and penicillin (100 U/ml)/streptomycin (100 μg/ml) at 37° C. in 5% CO₂. Cells were then seeded in 96-well plates at a 1.8×104 cells/well density. After approximately one day growth, cells were transfected with plasmids encoding nuclease and U6-guide RNA expression cassettes using FuGENE HD (Promega) per manufacturer's instruction. Briefly, on the day of transfection, 0.450 μg of plasmid DNA (0.353 μg of Cas-alpha nuclease and 0.099 μg of sgRNA expression cassettes) was diluted in a final volume of 20 μl of room temperature Opti-MEM (Gibco), mixed, and 1.35 μl of FuGENE HD was added directly to the solution (not allowing undiluted transfection reagent to contact the sides or bottom of the tube or plate) to achieve a 3:1 ratio of transfection reagent to plasmid DNA. The resulting mixture was then incubated for 10 mins at room temperature, 20 μl added to each HEK293T seeded well, and mixed gently by pipetting or by using a shaker. Transfected cells were then grown at 37° C. with 5% CO₂ for 4 days and GFP expression monitored by fluorescence microscope. Experiments were performed with three to twelve independent replicates and transfections where Cas-alpha 10 endonuclease and/or sgRNA plasmid DNA expression cassettes were omitted served as negative controls.

### Example 3: Detecting Cas-Alpha and Guide RNA Variants with Improved Cellular DNA Target Cleavage

In this Example, methods for detecting Cas-alpha endonuclease and guide RNA variants with improved double-stranded DNA target cleavage under low dosage or expression are described.

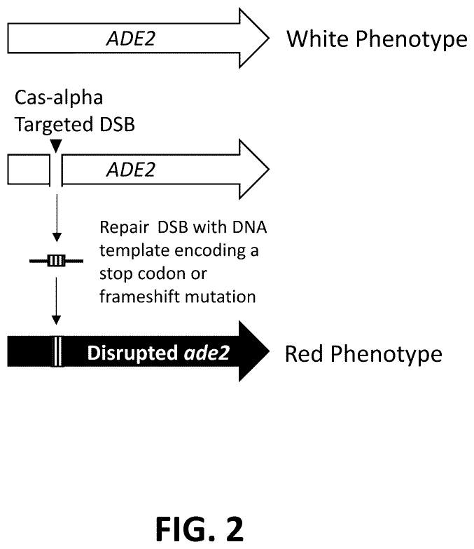

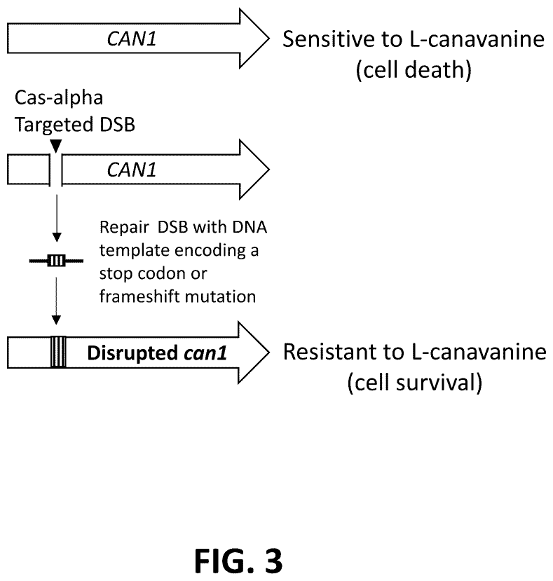

In one method, Cas-alpha nuclease variants and associated single guide RNA (sgRNA) were encoded on a low copy (2-5 copies per cell) Saccharomyces cerevisiae CEN6 autonomous replicating yeast plasmid DNA (Karim et al. (2013) FEMS Yeast Research. 13:107-116) and expressed using the ROX3 promoter, previously demonstrated to support weak gene expression (Generoso et al. (2016) Journal of Microbiological Methods. 127:203-205). Then by targeting either the ADE2 and/or CAN1 gene(s) in S. cerevisiae (BY4742, genotype—MATα his3Δ1 leu2Δ0 lys2Δ0 ura3Δ0) variants with improved cellular targeted DSB activity were selected. See FIGS. 2 and 3.

When targeting the ADE2 gene, selections for Cas-alpha nuclease and guide RNA target cleavage were done by monitoring the shift from white to red (pink) cellular phenotype resulting from the formation of a non-functional ade2 gene (FIG. 2). When viewed as a colony and depending on how quickly the ADE2 gene was disrupted, red coloration varied from entirely red to the observance of smaller red sector(s) within the otherwise white colony. These phenotypic patterns were also used to quantify differences in activity among variants. Colonies scoring as completely red, those containing several sectors, or just one red sector were counted and divided by the total number of colonies present. In some instances, instead of counting colonies, images of yeast colonies were captured with a Nikon Digital Sight Ds-Fil camera (Nikon, Japan) and NIS-Elements BR software (version 4.00.07) (Nikon, Japan) and analyzed by first determining the total yeast area (as pixels) and then calculating the total percentage of red using custom scripts.

For CAN1 experiments, Cas-alpha nuclease variants with improved targeted DSB activity were selected for by plating on media containing a toxic analog of arginine, L-canavanine. In this assay, target cleavage and non-functional repair of the CAN1 gene (FIG. 3) provides resistance against L-canavanine by disrupting its transport into the cell (Whelan et al. (1979) Genetics. 91: 35-51). Therefore, if CAN1 gene is cleaved and non-functionally repaired, L-canavanine is not transported across the cellular membrane and the cell survives; by contrast, if the CAN1 gene is not cleaved, L-canavanine enters resulting in cell death (FIG. 3). To modulate selective pressure, out-growth time in YPD media was varied between 2-7 hours before plating on L-canavanine (60 mg/mL). In some experiments, additional selection was also incorporated by adjusting the out-growth temperature between 30° C. and 37° C. depending on the Cas-alpha nuclease thermal preference and desired selective pressure.

In some instances, both ADE2 and CAN1 genes in S. cerevisiae were targeted for Cas-alpha endonuclease target cleavage. Here, disrupted ade2 red cellular phenotype and resistance to L-canavanine were both utilized to select for Cas-alpha nuclease variants with improved targeted DSB activity.

Once an improved Cas-alpha variant was identified, it was transferred to 5 mls of yeast broth without histidine (for example but not limited to 6.7 g/L yeast nitrogen base without amino acids (Becton Dickinson), 20 g/L glucose (Phytotechnology Labs) and 1.92 g/L yeast histidine dropout media (MP Biomedicals)) and incubated overnight at 30° C. with shaking. The CEN6 plasmid encoding the variant(s) was then isolated using a Yeast Plasmid Miniprep 96 kit (Zymo Research). After purification, the plasmid was next transformed into TransforMax EPI300 (Lucigen) E. coli competent cells per manufacturer's instruction and plated on selective media (for example but not limited to 6.7 g/L yeast nitrogen base without amino acids (Becton Dickinson), 20 g/L glucose (Phytotechnology Labs), 1.92 g/L yeast histidine dropout media (MP Biomedicals) and 20 g/L Bacto Agar (Becton Dickinson)). To account for the possibility that multiple CEN6 vectors were transformed and maintained in a single yeast cell, 4-6 colonies from each E. coli transformation were selected and each used to inoculate 2 mis of 2×YT medium (Sigma-Aldrich) or equivalent containing kanamycin. Cultures were then grown overnight at 37° C. with shaking and then half of the culture was subject to rolling circle amplification and sanger sequencing (Eurofins Scientific) using primers specific to the regions immediately adjacent to or within the cas-alpha gene. After sequencing, plasmid DNA was isolated from E. coli cultures (the remaining half) shown to contain different variants using a Qiagen Spin Miniprep Kit (Qiagen). Finally, ~1 μg of each plasmid was re-transformed back into S. cerevisiae and evaluated for a red cellular phenotype and/or L-canavanine resistance to confirm improvements. To make a comparison among improved variants and the Cas-alpha endonuclease subjected to directed evolution, an additional yeast transformation was typically performed with all variants and their ability to recognize and cleave the ADE2 and/or CAN1 target site reassessed.

In a second assay, Cas-alpha nuclease and sgRNA target cleavage efficiency was assessed by measuring the frequency of small insertion or deletion (indel) mutations resulting from the cellular repair of chromosomal DNA target cleavage in Zea mays or human cells. For this, experiments were performed similar to that described in Svitashev et al., 2015, Karvelis et al., 2015, and Bigelyte et al., 2021.

For Zea mays, two types of assays were used. In the first, transformed immature maize embryos were harvested 2-10 days after transformation, genomic DNA extracted, and Cas-alpha targets examined by Ampli-seq for the presence of mutations indicative of RNA-guided Cas-alpha editing. Briefly, the 20-30 most evenly transformed immature embryos, based on their fluorescence, were harvested for each experiment. Next, total genomic DNA was extracted and the region surrounding the intended target site was PCR amplified with Phusion® HighFidelity PCR Master Mix (New England Biolabs, M0531L) adding on the sequences necessary for amplicon-specific barcodes and Illumina sequencing using “tailed” primers through two rounds of PCR and deep sequenced. The resulting reads were then examined for the presence of mutations at the expected site of cleavage by comparison to control experiments where the small RNA expression cassette was omitted from the transformation. Sequence reads containing putative indels where further validated as true mutations by confirming their absence in the control datasets.

For the second Zea mays assay, regenerated plants were sampled, and DNA targets examined for evidence of editing using Ampli-Seq. This was performed as described above, although, to assess the likelihood of inheritance, the frequency of edited and wildtype sequence reads for each plant was also calculated. Since maize is diploid, plants with ~50% and ~100% mutant reads were assumed to be heterozygous and homozygous, respectively, for the targeted mutation (Zhang et al., (2014) Plant Biotechnology Journal. 12:797-807).

For human cells, cells were washed twice with PBS two to four days after transfection and lysed using lysis buffer (50 mM Tris, 150 mM NaCl, 0.2% Tween 20, 0.2 mg/ml Proteinase K). Next, RUNX and WTAP targets were evaluated for the presence of targeted mutations using T7 endonuclease I (Guschin, D. Y. et al. (2010) In: Mackay J., Segal D. (eds) Engineered Zinc Finger Proteins. Methods in Molecular Biology (Methods and Protocols), vol 649. Humana Press, Totowa, NJ) and Illumina deep sequencing as described above.

### Example 4: Library Design and Generation

In this Example, methods for designing and generating Cas-alpha endonuclease variants for improved double-stranded DNA target cleavage are described.

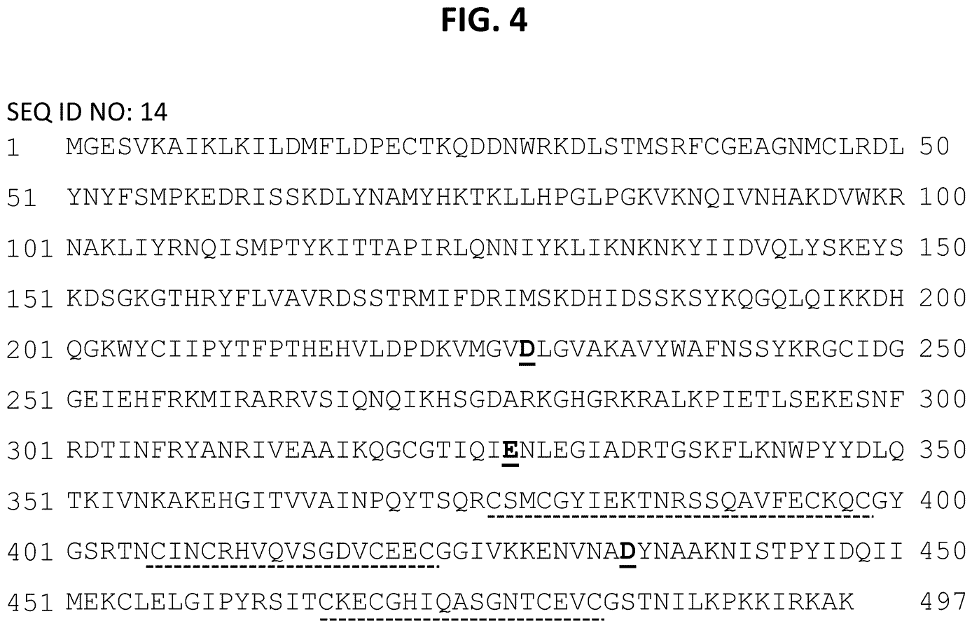

In one method, saturation mutagenesis was performed introducing all other amino acids (19 in total) at each position of the entire amino acid sequence of Cas-alpha 10 variant SEQ ID NO:14 (FIG. 4). In a second method, beneficial changes identified herein were incorporated (either individually or in combination) into variants already containing one or more beneficial changes and assayed for their combined effects. In a third method, amino acid substitutions were reverted to the wildtype residue in variants containing two or more changes, assayed for improvements in cleavage activity, and, if two or more beneficial reversions identified, combined and re-assayed for improvements.

For the first library method, codons within the cas-alpha nuclease gene in the yeast expression plasmid shown in FIG. 1 (SEQ ID NO:2) were altered to encode for different amino acids using GenPlus gene synthesis technology (GenScript). For library methods two and three, the cas-alpha nuclease gene encoding amino acid substitutions and/or reversions were produced by synthesis (GenScript) and/or introduced using site-directed mutagenesis as described earlier (Kunkel (1985) Proc Natl Acad Sci U.S.A. 82, 488-492).

### Example 5: Cas-Alpha Endonuclease Variants with Improved Cellular Activity

In this Example, Cas-alpha endonuclease variants with improved double-stranded DNA cleavage activity are described. All variants generated in Example 4 were tested. These included variants that had all possible amino acid substitutions across the entire length of the protein (positions 1-497, relative to SEQ ID NO: 14), variants containing a combination of beneficial alterations, and variants comprised of a combination of beneficial alterations with one or more amino acids reverted to a wildtype residue. Assays were conducted to capture variants that had improved desirable activity.

In Zea mays, amino acid substitutions giving rise to Cas-alpha 10 variant SEQ ID NO:14, A40G, E81G, A87K, T190K, T217H, K298S, H306F, T335R, and 1405N, were individually reverted to the wildtype residue (see equivalent position in SEQ ID NO:157) and assessed for their impact on targeted mutagenesis. In these experiments, immature embryos were kept at 28° C. until one day after particle-mediated transformation and then they were incubated at 33vC for 72 hrs and harvested for analysis. Additionally, plasmids encoding the guide RNA recognition domain from the Cas-alpha 10 sgRNA SEQ TD NO:61 targeting either the MS26 or CR36 site were co-delivered with a plasmid encoding the respective Cas-alpha 10 nuclease variant. As shown in Table 2, several amino acids (G81E, S298K, F306H, and N405I) when changed back to the wildtype residue resulted in an elevated frequency of targeted mutagenesi.

TABLE 2

| Amino Acid | Cas-alpha | | Average % | |
|---|---|---|---|---|
| Alteration to | Nuclease | Zea mays | Targeted | Standard |
| SEQ ID NO: 14 | SEQ ID NO: | Target Site | Mutagenesis | Deviation |
| None, Negative | 14 | None, guide | 0.00 | 0.00 |
| Control | | RNA expression | | |
| | | construct | | |
| | | omitted | | |
| None, | 14 | MS26 | 0.24 | 0.02 |
| Comparator 1 | | | | |
| None, | 14 | CR36 | 0.37 | 0.24 |
| Comparator 2 | | | | |
| G40A | 309 | MS26 | 0.22 | 0.01 |
| G81E | 48 | MS26 | 0.35 | 0.02 |
| K87A | 310 | MS26 | 0.19 | 0.02 |
| K190T | 311 | MS26 | 0.11 | 0.01 |
| H217T | 312 | CR36 | 0.32 | 0.21 |
| S298K | 313 | MS26 | 0.35 | 0.05 |
| F306H | 314 | MS26 | 0.31 | 0.09 |
| R335T | 315 | MS26 | 0.16 | 0.00 |
| N405I | 316 | MS26 | 0.28 | 0.10 |

The foregoing results identify a collection of amino acid substitutions that if reverted to a wildtype residue can be used to engineer Cas-alpha polypeptide variants that provide significantly improved DNA cleavage activity and targeting efficiency. Cas-alpha polypeptide variants with improved DNA cleavage activity and targeting efficiency include but are not limited to SEQ ID NOs: 48, 313-323, and 362.

In yeast, all possible amino acid substitutions across the entire length of the protein (positions 1-497, relative to SEQ ID NO: 14) were next screened for those that improved double-stranded DNA target cleavage activity when the Cas-alpha 10 nuclease and guide RNA (recognition domain from the Cas-alpha 10 original sgRNA (SEQ ID NO:61)) were encoded on a low copy CEN6 yeast plasmid DNA and the Cas nuclease was expressed from the weak ROX3 promoter. Initial selection was applied by targeting both ADE2 and CAN1 S. cerevisiae genes applying an out-growth temperature regime of 1 hr at 30° C. and then 2 hrs at 37° C. or 1 hr at 30° C. and then 4 hrs at 37° C. Following out-growth, cells were plated on media containing L-canavanine, placed at 37° C. overnight, and then grown at 30° C. until colony growth was visible. Variants that survived L-canavanine and exhibited an ade2 red cellular phenotype were selected and sequenced. To visualize activity relative to the starting Cas-alpha nuclease variant (FIG. 4, SEQ ID NO:14), experiments were next setup using only the ADE2 screen. For this, cells were plated on adenine deficient media immediately after transformation and placed at 37° C. overnight, and then grown at 30° C. until colony growth appeared (~2 days). Photographs were taken of the S. cerevisiae colonies and total percentage of ade2 red phenotype calculated by image analysis with custom scripts. The fold improvement in targeted DNA cleavage was calculated by dividing the total percentage of red S. cerevisiae produced with each variant by that observed from the original protein subject to directed evolution (FIG. 4, SEQ ID NO: 14) (Table 3).

TABLE 3

| Amino Acid | % Red | Fold Improvement in | SEQ ID |
|---|---|---|---|
| Alteration | Yeast | Target DNA Cleavage | NO: |
| None (original protein) | 31.09 | na | 18 |
| T34E | 59.21 | 1.90 | 19 |
| S36A | 46.48 | 1.50 | 20 |
| M45S | 49.72 | 1.60 | 21 |
| F54D | 80.08 | 2.58 | 22 |
| L77S | 44.38 | 1.43 | 23 |
| K85Q | 59.88 | 1.93 | 24 |

Combinations of the changes listed in Table 3 were next tested to identify those that provide additional improvement using the ADE2 screen with a 37° C. overnight incubation as described above. Certain combinations also incorporated a G81E reversion (SEQ ID NO:48) identified in Zea mays experiments. Based on visual assessment of ade2 red phenotype, several combinations were shown to further improve targeted DNA cleavage activity (Table 4). To further differentiate each variant, the 37° C. overnight incubation was omitted, and cells were incubated at 25° C. or 30° C. immediately after transformation until colonies were visible. As shown in Table 4, all combinations improved activity at 30° C.; and M45S+F54D+G81E+K85Q+G470D (SEQ ID NO: 50) provided the highest gain in activity (1.52-fold at 37° C., 29.05-fold at 30° C., and 29.58-fold at 25° C.) relative to the original protein (FIG. 4, SEQ TD NO: 14).

Table 4 lists the amino acid substitutions that in combination further improved double-stranded DNA target cleavage activity. S. cerevisiae experiments were performed as described above and fold improvement in targeted DNA cleavage calculated as described above relative to the original protein.

TABLE 4

| | | | | Fold Improvement | | | |
|---|---|---|---|---|---|---|---|
| | % Red Yeast | | | in Target DNA Cleavage at | | | SEQ ID |
| Amino Acid Alteration | 37° C. | 30° C. | 25° C. | 37° C. | 30° C. | 25° C. | NO: |
| None (original protein) | 59.89 | 3.02 | 2.29 | — | — | — | 14 |
| T34E + F54D | 86.73 | 65.53 | 14.36 | 1.45 | 21.73 | 6.26 | 31 |
| S36A + F54D | 84.88 | 56.87 | 8.54 | 1.42 | 18.86 | 3.73 | 32 |
| M45S + F54D | 78.02 | 54.99 | 4.47 | 1.30 | 18.23 | 1.95 | 33 |
| F54D + K85Q | 83.25 | 78.28 | 15.96 | 1.39 | 25.95 | 6.96 | 34 |
| F54D + G470D | 82.77 | 64.61 | 10.67 | 1.38 | 21.42 | 4.65 | 35 |
| F54D + L488E | 81.28 | 54.18 | 7.13 | 1.36 | 17.96 | 3.11 | 36 |
| T34E + F54D + K8 | 79.58 | 63.82 | 8.48 | 1.33 | 21.16 | 3.70 | 37 |
| S36A + F54D + K85Q | 82.11 | 62.07 | 10.32 | 1.37 | 20.58 | 4.50 | 38 |
| M45S + F54D + K85Q | 80.19 | 62.31 | 12.41 | 1.34 | 20.66 | 5.41 | 39 |
| F54D + K85Q + G470D | 82.7 | 69.99 | 12.41 | 1.38 | 23.20 | 5.41 | 40 |
| F54D + K85Q + L488E | 83.21 | 62.28 | 8.92 | 1.39 | 20.65 | 3.89 | 41 |
| T34E + F54D + K85Q + G470D | 82.17 | 81.28 | 16.50 | 1.37 | 26.95 | 7.20 | 42 |
| S36A + F54D + K85Q + G470D | 84.51 | 70.12 | 15.59 | 1.41 | 23.25 | 6.80 | 43 |
| M45S + F54D + K85Q + G470D | 83.15 | 71.00 | 8.26 | 1.39 | 23.54 | 3.60 | 44 |
| T34E + F54D + K85Q + L488E | 85.15 | 70.87 | 10.77 | 1.42 | 23.50 | 4.70 | 45 |
| S36A + F54D + K85Q + L488E | 80.07 | 62.41 | 8.41 | 1.34 | 20.69 | 3.67 | 46 |
| M45S + F54D + K85Q + L488E | 80.24 | 53.64 | 8.74 | 1.34 | 17.78 | 3.81 | 47 |
| T34E + F54D + G81E + | 88.89 | 87.00 | 44.62 | 1.48 | 28.84 | 19.46 | 49 |
| K85Q + G470D | | | | | | | |
| M45S + F54D + G81E + | 91.21 | 87.61 | 67.82 | 1.52 | 29.05 | 29.58 | 50 |
| K85Q + G470D | | | | | | | |
| T34E + M45S + F54D + | 89.99 | 86.03 | 49.54 | 1.50 | 28.52 | 21.61 | 51 |
| K85Q + G470D | | | | | | | |
| T34E + S36A + F54D + | 93.20 | 87.09 | 22.42 | 1.56 | 28.87 | 9.78 | 52 |
| K85Q + G470D | | | | | | | |
| T34E + S36A + F54D + G81E + | 76.73 | 86.74 | 34.08 | 1.28 | 28.76 | 14.87 | 53 |
| K85Q + G470D | | | | | | | |
| T34E + M45S + F54D + G81E + | 82.07 | 88.66 | 58.06 | 1.37 | 29.39 | 25.32 | 54 |
| K85Q + G470D | | | | | | | |
| T34E + S36A + M45S + F54D + | 87.10 | 87.29 | 57.26 | 1.45 | 28.94 | 24.98 | 55 |
| G81E + K85Q + G470D | | | | | | | |
| T34E + S36A + M45S + F54D + | 88.43 | 86.80 | 28.55 | 1.48 | 28.78 | 12.45 | 56 |
| K85Q + G470D | | | | | | | |

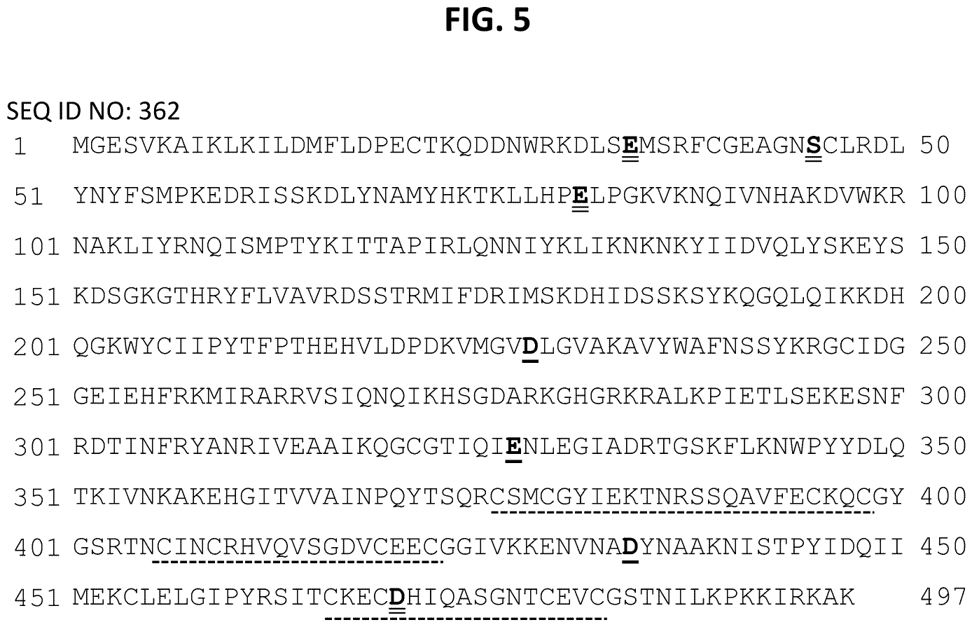

K85Q was also predicted with AlphaFold to be involved in PAM recognition. Therefore, it was converted back to a K residue for M45S+F54D+G81E+K85Q+G470D (SEQ ID NO: 50) and T34E+M45S+F54D+G81E+K85Q+G470D (SEQ ID NO: 54) and re-tested using two ADE2 target sites. Testing was performed as described above and fold improvement in targeted DNA cleavage was calculated as described above, relative to the original protein. Results shown in Table 5 indicate that changing the K residue had a negligible effect on activity at the initial ADE2 target with a 5′-ATTC-3′ PAM (ADE2-CR2, SEQ ID NO: 57). However, when used for the second ADE2 target site with a 5′-GTTC-3′ PAM (ADE2-CR1, SEQ ID NO: 58), the restored K residue substantially improved activity, as shown in Table 5, with T34E+M45S+F54D+G81E+G470D (SEQ ID NO: 60) (FIG. 5) demonstrating the largest fold improvement across both targets.

TABLE 5

| | | | Fold | | |
|---|---|---|---|---|---|
| | | | Improvement in | | |
| | % Red Yeast | | Target DNA | | |
| | 37° C. | | Cleavage at | | SEQ |
| | ADE2- | ADE2- | ADE2- | ADE2- | ID |
| Amino Acid Alteration | CR2 | CR1 | CR2 | CR1 | NO: |
| None (original protein) | 45.53 | 2.76 | — | — | 14 |
| M45S + F54D + G81E + | 91.03 | 5.19 | 2.00 | 1.88 | 50 |
| K85Q + G470D | | | | | |
| T34E + M45S + F54D + | 87.99 | 7.48 | 1.93 | 2.72 | 54 |
| G81E + K85Q + G470D | | | | | |
| M45S + F54D + | 85.59 | 45.38 | 1.88 | 16.46 | 59 |
| G81E + G470D | | | | | |
| T34E + M45S + F54D + | 87.34 | 64.52 | 1.92 | 23.40 | 60 |
| G81E + G470D | | | | | |

Additionally, F54D was predicted using AlphaFold to potentially alter the oligomerization state of Cas-alpha 10 and was changed back to a phenylalanine. The targeted mutation efficiency of the new variant, T34E+M45S+G81E+G470D (SEQ ID NO:362), was then compared with T34E+M45S+F54D+G81E+G470D (SEQ ID NO:60) in human cells (at 37° C.). As shown in Table 6, T34E+M45S+G81E+G470D (SEQ ID NO:362) showed enhanced targeted mutagenesis at the DNMT and WTAP sites and performed equivalently to T34E+M45S+G81E+G470D (SEQ ID NO:362) at the RUNX target.

TABLE 6

| Cas-alpha 10 | Target | % Targeted | Standard |
|---|---|---|---|
| Nuclease | Site | Mutagenesis | Deviation |
| 60 | DNMT | 64.20 | 5.83 |
| 362 | | 74.05 | 4.18 |
| 60 | WTAP | 14.24 | 1.62 |
| 362 | | 41.69 | 9.24 |
| 60 | RUNX | 18.39 | 1.65 |
| 362 | | 20.23 | 1.62 |

The double-stranded DNA target cleavage activity of SEQ ID NO:157, SEQ ID NO:14, SEQ ID NO:48, and SEQ ID NO:362 were next evaluated biochemically similar to that described in Gasiunas et al. (2020) Nat Commun. 11:5512. Briefly, Cas-alpha 10 protein was expressed and purified as described in Bigelyte et al., 2021 and sgRNA produced using HiScribe T7 Quick High Yield RNA Synthesis Kits (NEB). sgRNA was then diluted in 1× Reaction Buffer (Tris, pH7.5, 200 nM NaCl, 1 mM DTT, 1 mM EDTA, 10 mM MgCl₂, and 0.01 mM ZnCl₂) and heat denatured at 90° C. for 3 min. and then cooled slowly to 12° C. Next, sgRNA and purified Cas-alpha 10 nuclease pre-diluted in 1× Reaction Buffer were combined, incubated for 5-10 min. at room temperature, and then moved to the desired reaction temperature for 2-3 min. before mixing with 5′-FAM labeled double-strand DNA substrate. Reactions were performed in 10 μl of 1× Reaction Buffer with final concentrations of sgRNA, Cas-alpha nuclease, and DNA substrate of 200 nM, 200 nM, and 10 nM, respectively. After 30 min., reactions were quenched using stop buffer (1× reaction buffer, 67.5 mM EDTA (final concentration), 0.1 U/μl of Proteinase K (final concentration) and the extent of target cleavage assayed using capillary electrophoresis after diluting 4× in nuclease-free water.

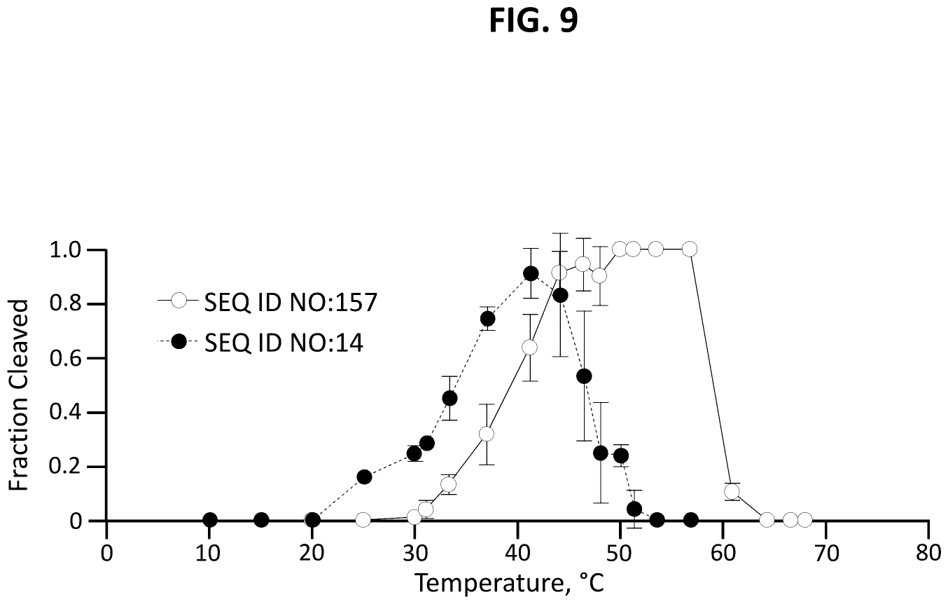

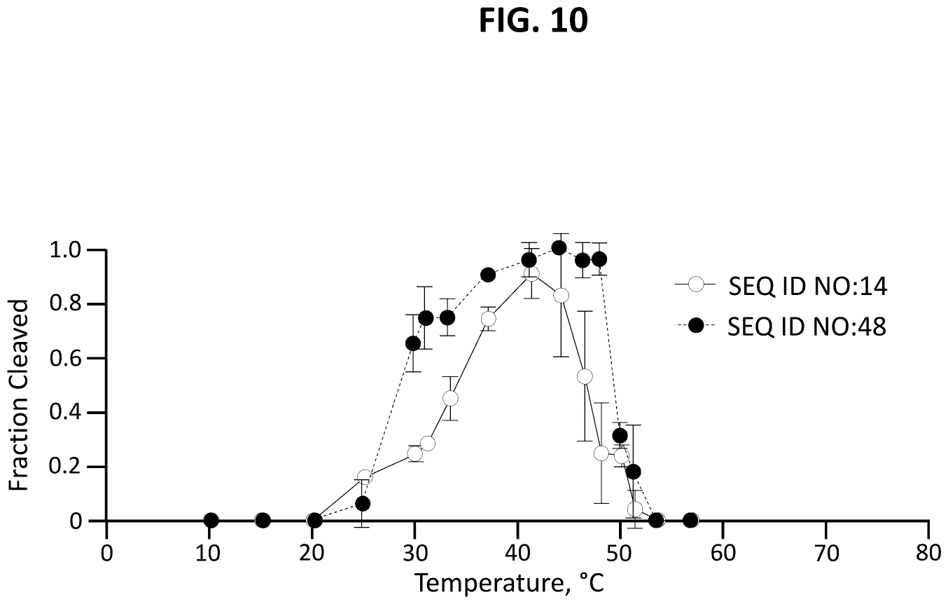

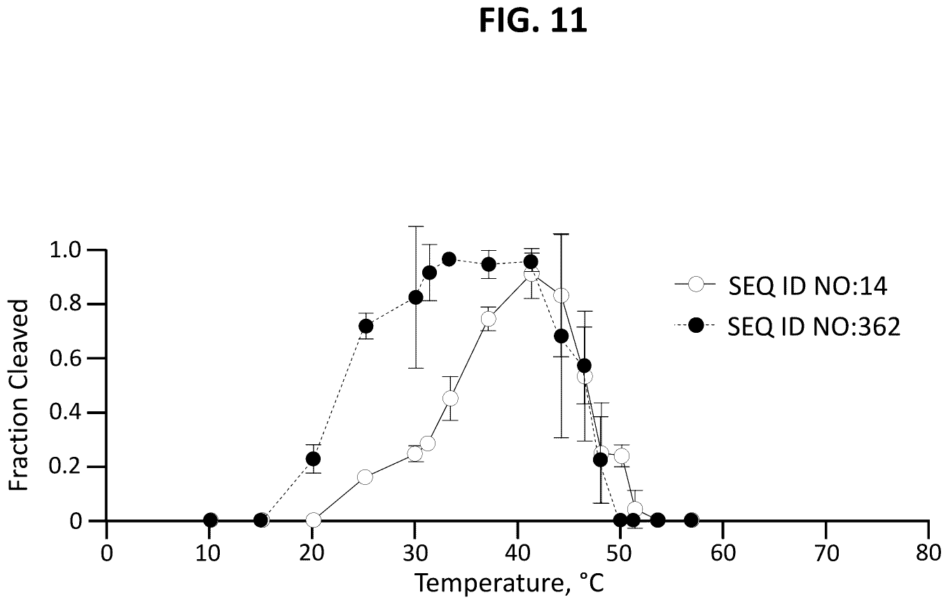

The effect of temperature on nuclease activity was evaluated initially. As shown in FIG. 9, the wildtype Cas-alpha 10 nuclease (SEQ ID NO:157) cleaved its double-stranded DNA substrate robustly (>0.6 fraction of targets cleaved) at temperatures ranging from 42 to 58° C. This can be contrasted with SEQ ID NO:14, SEQ ID NO:48, and SEQ ID NO:362 whose requirement for elevated temperatures was decreased (FIGS. 9-11). Moreover, as a result of the engineering described herein, the range of temperatures that SEQ ID NO:48 efficiently cleaved its target was expanded to 30-48° C. compared to SEQ ID NO:14 which had more narrow temperature preferences, 37-43° C. (FIG. 10). Finally, the thermal profile for SEQ ID NO:362 was further shifted towards lower temperatures demonstrating efficient substrate cleavage at temperatures as low as 25° C. (FIG. 11).

The foregoing results identify a collection of amino acid alterations that can be used to engineer Cas-alpha polypeptide variants that provide significantly improved DNA cleavage activity, targeting efficiency, and/or PAM preference. The improved function of the disclosed variants is notable relative to the starting Cas-alpha variant (FIG. 4, SEQ ID NO:14). The results also show that the disclosed variants and amino acid alterations can broaden the range of possible applications and scope of organisms in which such engineered Cas-alpha polypeptide variants may be used as Cas effectors, e.g., endonucleases, including for targeted genome editing.

### Example 6: Cas-Alpha sgRNA Variants with Improved Cellular Activity

In this Example, Cas-alpha sgRNA variants with improved double-stranded DNA cleavage activity are described.

In one method, three nucleotides of the Cas-alpha 10 sgRNA recognition domain (SEQ ID NO: 61) were systematically deleted in one nucleotide overlapping intervals and its ability to cleave the ADE2 gene was evaluated as described above in Example 5, except that two yeast plasmids were used. The first contained a CEN6 ORI and a Cas-alpha 10 endonuclease (SEQ ID NO: 14) expression cassette while the second constituted a 2-micron ORI and the sgRNA expression cassette. DNA sequences encoding sgRNA variants were produced by inverse PCR and seamless cloning using the 2-micron plasmid encoding a sgRNA (SEQ ID NO: 62) capable of directing Cas-alpha 10 endonuclease cleavage at the ADE2-CR2 target site as template in S. cerevisiae. Experiments were performed at 37° C. and percentage of red cellular phenotype resulting from ADE2-CR2 target cleavage was normalized to the original Cas-alpha sgRNA (SEQ ID NO: 62). Values of about 1.00 represent comparable activity to the original sgRNA while those above or below about 1.00 correspond to an increase or decrease, respectively, in ADE2-CR2 target cleavage. The results of deleting different three nucleotide regions of the Cas-alpha 10 sgRNA recognition domain are shown in Table 7.

TABLE 7

| Region of Cas-alpha | % Red Yeast | SEQ |
|---|---|---|
| 10 sgRNA Recognition | Normalized to Original | ID |
| Domain Deleted | Guide RNA | NO: |
| 1-3 | 0.90 | 63 |
| 3-5 | 2.05 | 64 |
| 5-7 | 1.90 | 65 |
| 7-9 | 1.64 | 66 |
| 9-11 | 0.29 | 67 |
| 11-13 | 0.43 | 68 |
| 13-15 | 0.42 | 69 |
| 15-17 | 0.14 | 70 |
| 17-19 | 0.08 | 71 |
| 19-21 | 0.44 | 72 |
| 21-23 | 0.02 | 73 |
| 23-25 | 0.02 | 74 |
| 25-27 | 0.02 | 75 |
| 27-29 | 0.04 | 76 |
| 29-31 | 0.02 | 77 |
| 31-33 | 0.03 | 78 |
| 33-35 | 0.08 | 79 |
| 35-37 | 0.05 | 80 |
| 37-39 | 0.02 | 81 |
| 39-41 | 0.24 | 82 |
| 41-43 | 0.09 | 83 |
| 43-45 | 0.02 | 84 |
| 45-47 | 0.02 | 85 |
| 47-49 | 0.40 | 86 |
| 49-51 | 0.01 | 87 |
| 51-53 | 0.04 | 88 |
| 53-55 | 0.01 | 89 |
| 55-57 | 0.06 | 90 |
| 57-59 | 0.84 | 91 |
| 59-61 | 2.70 | 92 |
| 61-63 | 2.29 | 93 |
| 63-65 | 1.34 | 94 |
| 65-67 | 0.33 | 95 |
| 67-69 | 0.14 | 96 |
| 69-71 | 0.02 | 97 |
| 71-73 | 0.08 | 98 |
| 73-75 | 0.05 | 99 |
| 75-77 | 0.09 | 100 |
| 77-79 | 0.01 | 101 |
| 79-81 | 0.11 | 102 |
| 81-83 | 0.07 | 103 |
| 83-85 | 0.06 | 104 |
| 85-87 | 0.06 | 105 |
| 87-89 | 0.08 | 106 |
| 89-91 | 0.68 | 107 |
| 91-93 | 0.18 | 108 |
| 93-95 | 0.03 | 109 |
| 95-97 | 0.04 | 110 |
| 97-99 | 0.00 | 111 |
| 99-101 | 0.00 | 112 |
| 101-103 | 0.02 | 113 |
| 103-105 | 0.01 | 114 |
| 105-107 | 0.00 | 115 |
| 107-109 | 0.00 | 116 |
| 109-111 | 0.00 | 117 |
| 111-113 | 0.00 | 118 |
| 113-115 | 0.00 | 119 |
| 115-117 | 0.00 | 120 |
| 117-119 | 0.00 | 121 |
| 119-121 | 0.00 | 122 |
| 121-123 | 0.02 | 123 |
| 123-125 | 0.02 | 124 |
| 125-127 | 0.05 | 125 |
| 127-129 | 0.02 | 126 |
| 129-131 | 0.06 | 127 |
| 131-133 | 0.22 | 128 |
| 133-135 | 0.02 | 129 |
| 135-137 | 0.21 | 130 |
| 137-139 | 0.08 | 131 |
| 139-141 | 0.11 | 132 |
| 141-143 | 0.16 | 133 |
| 143-145 | 0.10 | 134 |
| 145-147 | 0.19 | 135 |
| 147-149 | 0.92 | 136 |
| 149-151 | 1.08 | 137 |
| 151-153 | 1.13 | 138 |
| 153-155 | 1.30 | 139 |
| 155-157 | 0.61 | 140 |
| 157-159 | 2.20 | 141 |
| 159-161 | 0.97 | 142 |
| 161-163 | 0.59 | 143 |
| 163-165 | 1.14 | 144 |
| 165-167 | 0.37 | 145 |
| 167-169 | 1.39 | 146 |
| 169-171 | 0.87 | 147 |
| 171-173 | 0.21 | 148 |
| 173-175 | 0.26 | 149 |
| 175-177 | 0.00 | 150 |
| 177-179 | 0.00 | 151 |
| 179-181 | 0.00 | 152 |
| 181-183 | 0.00 | 153 |
| 183-185 | 0.00 | 154 |
| 185-187 | 0.00 | 155 |
| 187-189 | 0.02 | 156 |

The foregoing results (percentage of red yeast) in Table 7 show that three-nucleotide deletions were tolerated at positions 1-3, 3-5, 5-7, 7-9, 59-61, 61-63, 63-65, 147-149, 149-151, 151-153, 153-155, 157-159, 159-161, 163-165, and 167-169. Notably deletions at 3-5, 5-7, 7-9, 59-61, 61-63, 63-65, 149-151, 151-153, 153-155, 157-159, 163-165, and 167-169 resulted in a gain in target cleavage efficiency at the ADE2-CR2 site.

Deletions in the sgRNA recognition domain that improved DNA target cleavage activity or had a negligible effect on it were next combined and tested for improvement in human cells. They were introduced into the recognition domain of four sgRNA designs, Cas-alpha 10 original sgRNA (SEQ TD NO:61), Cas-alpha 10 sgRNA MS 10 (SEQ TD NO:324), Cas-alpha 10 sgRNA MS 13 (SEQ TD NO:325), and Cas-alpha 10 sgRNA design 5 (SEQ TD NO:310 in WO-2022082179-A2)(SEQ ID NO:326)) as listed in Tables 8 and 9. Since Cas-alpha 10 sgRNA MS 10, Cas-alpha 10 sgRNA MS 13, and Cas-alpha 10 sgRNA design 5 already contained deletions at positions 1-3, 3-5, 5-7, 7-9, 147-149, 149-151, and 1S1-153, removal of these nucleotides were not needed (Tables 8 and 9). Additionally, deletions at positions 157-159 and 167-169 were not tested with Cas-alpha 10 sgRNA MS513 as the region encompassing these deletions wasn't present (Tables 8 and 9). Moreover, the MS13 solution was further modified in some instances by replacing the sequence linking the tracrRNA and crRNA portions or the sgRNA with a 5′-GAAA-3′ tetraloop (SEQ ID NO:327)(Tables 8 and 9).

At the RUNX target, new sgRNA solutions originating from the design 5 sgRNA had the largest effect on enhancing targeted indel frequencies in combination with Cas-alpha 10 endonuclease variants, SEQ TD NO:14 and SEQ TD NO:362, where highest frequencies of mutagenesis were obtained using design 5_157-159 (Table 8).

TABLE 8

| | | | | % | |
|---|---|---|---|---|---|
| Cas- | | Region of | Corres- | Targeted | |
| alpha | | Cas-alpha | ponding | Muta- | |
| 10 | | 10 sgRNA | sgRNA | genesis | |
| Nucle- | sgRNA | Recognition | (SEQ | at RUNX | Standard |
| ase | Name | Domain Deleted | ID NO:) | Site | Deviation |
| 157 | Original | NA | 192 | 5.68 | 3.26 |
| 157 | MS10 | 1-9, 146-153 | 193 | 5.68 | 3.19 |
| 157 | MS10_5 | 1-9, 59-61, 146- | 334 | 4.97 | 1.03 |
| | 9-61 | 153 | | | |
| 157 | MS13 | 1-9, 146-153, | 194 | 6.35 | 1.99 |
| | | 158-170 | | | |
| 157 | MS13_5 | 1-9, 59-61, 146- | 335 | 5.66 | 1.60 |
| | 9-61 | 153, 158-170 | | | |
| 157 | MS13_G | 1-9, 146-153, | 195 | 5.86 | 2.82 |
| | AAA | 158-170 | | | |
| 157 | MS13_G | 1-9, 146-153 | 196 | 5.66 | 1.60 |
| | AAA_59- | | | | |
| | 61 | | | | |
| 157 | TR144 | 1-9, 61-63, and | 197 | 6.22 | 2.55 |
| | | 157-159 | | | |
| 157 | TR149 | 1-9, 61-63, and | 198 | 5.78 | 2.77 |
| | | 167-169 | | | |
| 157 | TR150 | 1-9, 6-63, 157- | 199 | 5.07 | 2.34 |
| | | 159, and 167-169 | | | |
| 157 | Design 5 | 1-9 and 146-153 | 336 | 6.31 | 0.77 |
| 157 | Design | 1-9, 59-61, and | 337 | 5.46 | 0.63 |
| | 5_59-61 | 146-153 | | | |
| 157 | Design | 1-9, 61-63, and | 338 | 5.49 | 1.98 |
| | 5_61-63 | 146-153 | | | |
| 157 | Design | 1-9, 146-153, and | 339 | 5.40 | 3.55 |
| | 5_157- | 157-159 | | | |
| | 159 | | | | |
| 14 | Original | NA | 192 | 8.77 | 2.91 |
| 14 | MS10 | 1-9, 146-153 | 193 | 10.22 | 4.84 |
| 14 | MS10_5 | 1-9, 59-61, 146- | 334 | 6.44 | 1.98 |
| | 9-61 | 153 | | | |
| 14 | MS13 | 1-9, 146-153, | 194 | 11.09 | 5.58 |
| | | 158-170 | | | |
| 14 | MS13_5 | 1-9, 59-61, 146- | 335 | 6.15 | 2.22 |
| | 9-61 | 153, 158-170 | | | |
| 14 | MS13_G | 1-9, 146-153, | 195 | 8.18 | 3.17 |
| | AAA | 158-170 | | | |
| 14 | MS13_G | 1-9, 146-153 | 196 | 7.90 | 0.75 |
| | AAA_59- | | | | |
| | 61 | | | | |
| 14 | TR144 | 1-9, 61-63, and | 197 | 10.88 | 3.11 |
| | | 157-159 | | | |
| 14 | TR149 | 1-9, 61-63, and | 198 | 8.56 | 1.49 |
| | | 167-169 | | | |
| 14 | TR150 | 1-9, 6-63, 157- | 199 | 9.12 | 1.30 |
| | | 159, and 167-169 | | | |
| 14 | Design 5 | 1-9 and 146-153 | 336 | 13.66 | 1.85 |
| 14 | Design | 1-9, 59-61, and | 337 | 13.71 | 2.83 |
| | 5_59-61 | 146-153 | | | |
| 14 | Design | 1-9, 61-63, and | 338 | 15.28 | 2.08 |
| | 5_61-63 | 146-153 | | | |
| 14 | Design | 1-9, 146-153, and | 339 | 19.64 | 5.85 |
| | 5_157- | 157-159 | | | |
| | 159 | | | | |
| 362 | Original | NA | 192 | 10.53 | 3.31 |
| 362 | MS10 | 1-9, 146-153 | 193 | 14.89 | 5.24 |
| 362 | MS10_5 | 1-9, 59-61, 146- | 334 | 10.12 | 3.13 |
| | 9-61 | 153 | | | |
| 362 | MS13 | 1-9, 146-153, | 194 | 18.59 | 8.44 |
| | | 158-170 | | | |
| 362 | MS13_5 | 1-9, 59-61, 146- | 335 | 7.33 | 4.46 |
| | 9-61 | 153, 158-170 | | | |
| 362 | MS13_G | 1-9, 146-153, | 195 | 12.45 | 2.79 |
| | AAA | 158-170 | | | |
| 362 | MS13_G | 1-9, 146-153 | 196 | 11.80 | 1.05 |
| | AAA_59- | | | | |
| | 61 | | | | |
| 362 | TR144 | 1-9, 61-63, and | 197 | 17.00 | 9.74 |
| | | 157-159 | | | |
| 362 | TR149 | 1-9, 61-63, and | 198 | 13.49 | 6.15 |
| | | 167-169 | | | |
| 362 | TR150 | 1-9, 6-63, 157- | 199 | 16.48 | 10.21 |
| | | 159, and 167-169 | | | |
| 362 | Design 5 | 1-9 and 146-153 | 336 | 18.87 | 1.41 |
| 362 | Design | 1-9, 59-61, and | 337 | 19.47 | 4.80 |
| | 5_59-61 | 146-153 | | | |
| 362 | Design | 1-9, 61-63, and | 338 | 20.54 | 2.84 |
| | 5_61-63 | 146-153 | | | |
| 362 | Design | 1-9, 146-153, and | 339 | 24.20 | 8.99 |
| | 5_157- | 157-159 | | | |
| | 159 | | | | |

At the WTAP site, the design 5 sgRNA yielded the highest targeted mutation frequencies when paired with the Cas-alpha 10 endonucleases SEQ TD NO: 14 and 3362 (Table 8). When used in combination with the Cas-alpha 10 nuclease SEQ TD NO: 157, design 5_157-159 produced the highest rate of targeted mutagenesis (Table 9).

TABLE 9

| | | Region of | | % | |
|---|---|---|---|---|---|
| | | Cas-alpha | Corres- | Targeted | |
| Cas- | | 10 sgRNA | ponding | Muta- | |
| alpha | | Recognition | sgRNA | genesis | |
| 10 | sgRNA | Domain | (SEQ | at WTAP | Standard |
| Nuclease | Name | Deleted | ID NO:) | Site | Deviation |
| 157 | Original | NA | 200 | 6.11 | 5.47 |
| 157 | MS10 | 1-9, 146-153 | 201 | 12.62 | 5.86 |
| 157 | MS10_5 | 1-9, 59-61, 146- | 346 | 9.32 | 4.71 |
| | 9-61 | 153 | | | |
| 157 | MS13 | 1-9, 146-153, | 202 | 14.69 | 7.13 |
| | | 158-170 | | | |
| 157 | MS13_5 | 1-9, 59-61, 146- | 347 | 11.32 | 7.21 |
| | 9-61 | 153, 158-170 | | | |
| 157 | MS13_G | 1-9, 146-153, | 203 | 15.36 | 8.02 |
| | AAA | 158-170 | | | |
| 157 | MS13_G | 1-9, 146-153 | 204 | 10.38 | 11.32 |
| | AAA_59- | | | | |
| | 61 | | | | |
| 157 | TR144 | 1-9, 61-63, and | 205 | 20.50 | 9.01 |
| | | 157-159 | | | |
| 157 | TR149 | 1-9, 61-63, and | 206 | 15.09 | 7.36 |
| | | 167-169 | | | |
| 157 | TR150 | 1-9, 6-63, 157- | 207 | 18.36 | 10.15 |
| | | 159, and 167-169 | | | |
| 157 | Design 5 | 1-9 and 146-153 | 348 | 19.55 | 1.70 |
| 157 | Design | 1-9, 59-61, and | 349 | 19.27 | 3.37 |
| | 5_59-61 | 146-153 | | | |
| 157 | Design | 1-9, 61-63, and | 350 | 18.86 | 4.21 |
| | 5_61-63 | 146-153 | | | |
| 157 | Design | 1-9, 146-153, and | 351 | 25.79 | 6.35 |
| | 5_157- | 157-159 | | | |
| | 159 | | | | |
| 14 | Original | NA | 200 | 26.54 | 10.25 |
| 14 | MS10 | 1-9, 146-153 | 201 | 45.35 | 11.72 |
| 14 | MS10_5 | 1-9, 59-61, 146- | 346 | 41.51 | 9.11 |
| | 9-61 | 153 | | | |
| 14 | MS13 | 1-9, 146-153, | 202 | 43.02 | 16.04 |
| | | 158-170 | | | |
| 14 | MS13_5 | 1-9, 59-61, 146- | 347 | 34.29 | 8.93 |
| | 9-61 | 153, 158-170 | | | |
| 14 | MS13_G | 1-9, 146-153, | 203 | 37.68 | 14.83 |
| | AAA | 158-170 | | | |
| 14 | MS13_G | 1-9, 146-153 | 204 | 32.36 | 10.02 |
| | AAA_59- | | | | |
| | 61 | | | | |
| 14 | TR144 | 1-9, 61-63, and | 205 | 54.31 | 12.49 |
| | | 157-159 | | | |
| 14 | TR149 | 1-9, 61-63, and | 206 | 52.95 | 12.78 |
| | | 167-169 | | | |
| 14 | TR150 | 1-9, 6-63, 157- | 207 | 47.93 | 11.70 |
| | | 159, and 167-169 | | | |
| 14 | Design 5 | 1-9 and 146-153 | 348 | 67.77 | 4.21 |
| 14 | Design | 1-9, 59-61, and | 349 | 52.77 | 27.12 |
| | 5_59-61 | 146-153 | | | |
| 14 | Design | 1-9, 61-63, and | 350 | 47.08 | 17.45 |
| | 5_61-63 | 146-153 | | | |
| 14 | Design | 1-9, 146-153, and | 351 | 55.07 | 14.70 |
| | 5_157- | 157-159 | | | |
| | 159 | | | | |
| 362 | Original | NA | 200 | 30.67 | 13.49 |
| 362 | MS10 | 1-9, 146-153 | 201 | 54.42 | 15.66 |
| 362 | MS10_5 | 1-9, 59-61, 146- | 346 | 41.47 | 16.39 |
| | 9-61 | 153 | | | |
| 362 | MS13 | 1-9, 146-153, | 202 | 49.64 | 17.93 |
| | | 158-170 | | | |
| 362 | MS13_5 | 1-9, 59-61, 146- | 347 | 13.95 | 8.47 |
| | 9-61 | 153, 158-170 | | | |
| 362 | MS13_G | 1-9, 146-153, | 203 | 39.40 | 16.54 |
| | AAA | 158-170 | | | |
| 362 | MS13_G | 1-9, 146-153 | 204 | 42.17 | 18.69 |
| | AAA_59- | | | | |
| | 61 | | | | |
| 362 | TR144 | 1-9, 61-63, and | 205 | 52.86 | 17.15 |
| | | 157-159 | | | |
| 362 | TR149 | 1-9, 61-63, and | 206 | 53.15 | 14.88 |
| | | 167-169 | | | |
| 362 | TR150 | 1-9, 6-63, 157- | 207 | 46.30 | 16.87 |
| | | 159, and 167-169 | | | |
| 362 | Design 5 | 1-9 and 146-153 | 348 | 66.80 | 6.88 |
| 362 | Design | 1-9, 59-61, and | 349 | 49.28 | 20.14 |
| | 5_59-61 | 146-153 | | | |
| 362 | Design | 1-9, 61-63, and | 350 | 53.82 | 16.02 |
| | 5_61-63 | 146-153 | | | |
| 362 | Design | 1-9, 146-153, and | 351 | 51.76 | 20.32 |
| | 5_157- | 157-159 | | | |
| | 159 | | | | |

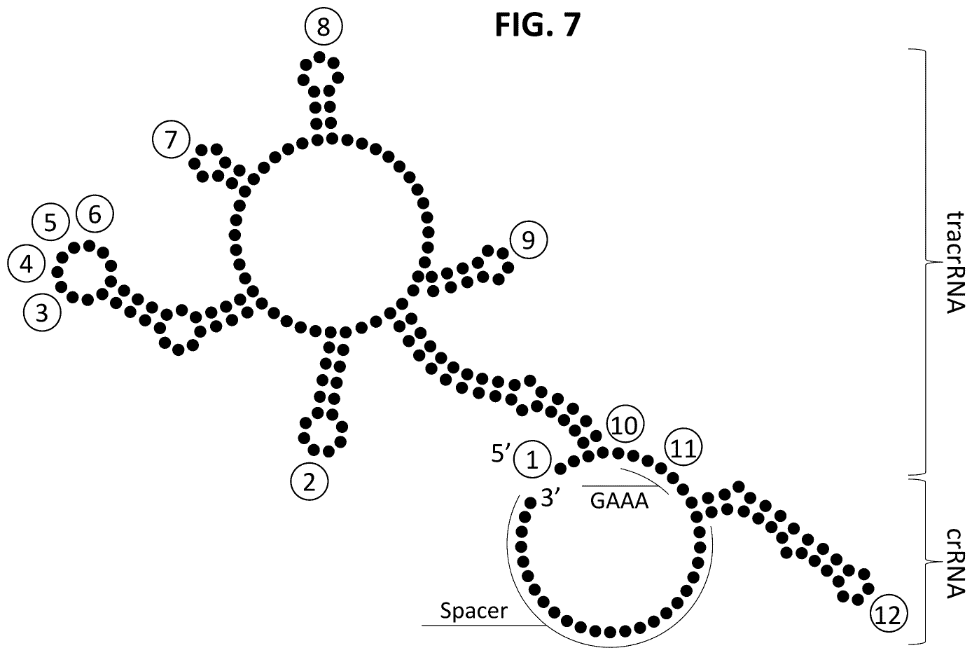

In a second study, RNA polypeptides were added to the Cas-alpha sgRNA. For example, sequences that formed hairpin-like secondary structures were incorporated, including hairpin-like RNA recognition sequence of the MS2 bacteriophage coat protein (SEQ ID NO: 208). Secondary structure of the Cas-alpha sgRNA was predicted using Vienna fold (Gruber et al. (2008) Nucleic Acids Res. 36: W70-74). Then, MS2 recognition sequences were incorporated at the 5′ end, the apex of stem-loops, and within the 5′-GAAA-3′ linker connecting the tracrRNA and crRNA of the sgRNA as shown in FIG. 7. To facilitate proper folding of the aptamer, additional sequences (for example G or C nucleotides on either side of the MS2 recognition sequence arranged in such a way to base pair with each other) were incorporated at the base of each sequence addition. Solutions were tested in human cells at one target in the DNMT gene for their ability to guide Cas-alpha endonuclease target recognition and cleavage using the evolved Cas-alpha 10 nuclease SEQ ID NO: 14. Three independent transfections were performed for each sgRNA variant; negative controls included experiments where Cas-alpha 10 endonuclease and sgRNA plasmid DNA expression cassettes were omitted.

As shown in Table 10, sgRNAs incorporating the MS2 aptamer were tolerated at positions 1, 3, 5, 7, 10, 11, and 12 with an improvement in targeted mutagenesis observed at position 1 relative to the original sgRNA. To confirm the results were not sequence dependent, the hairpin forming RNA recognition sequence of the Cas6 protein from Pseudomonas aeruginosa (SEQ ID NO: 209) (Stenberg et al. (2012) RNA. 18: 661-672) was also incorporated into the Cas-alpha sgRNA at the same positions and tested. A similar trend in targeted mutagenesis was observed with positions 1, 3, 4, 5, 6, 7, 9, 10, and 11 showing equivalent or better frequencies of targeted mutagenesis than the original sgRNA solution (Table 10).

TABLE 9

| | Region of | | | |
|---|---|---|---|---|
| | Cas-alpha | | | |
| | 10 sgRNA | | % Targeted | |
| | where | | Mutagenesis | |
| | MS2 or Cas6 | Corresponding | at | |
| sgRNA | Aptamer was | sgRNA (SEQ | DNMT | Standard |
| Name | Inserted | ID NO:) | Site | Deviation |
| Original | NA | 260 | 56.90 | 7.55 |
| MS2_POS1 | 5′ of position 1 | 261 | 82.53 | 5.16 |
| MS2_POS2 | 28-33 | 262 | 0.98 | 1.24 |
| MS2_POS3 | 57-65 | 263 | 56.45 | 4.43 |
| MS2_POS4 | 57-65 | 264 | 40.94 | 10.91 |
| MS2_POS5 | 59-61 | 265 | 57.81 | 6.89 |
| MS2_POS6 | 61-63 | 266 | 31.05 | 3.72 |
| MS2_POS7 | 86-89 | 267 | 50.04 | 7.23 |
| MS2_POS8 | 101-105 | 268 | 11.10 | 5.18 |
| MS2_POS9 | 127-130 | 269 | 37.11 | 11.92 |
| MS2_POS10 | 151-154 | 270 | 62.55 | 14.95 |
| MS2_POS11 | 157-159 | 271 | 60.46 | 11.22 |
| MS2_POS12 | Between | 272 | 48.56 | 15.25 |
| | 170-171 | | | |
| CAS6 POS1 | 5′ of position 1 | 273 | 69.62 | 14.80 |
| CAS6 POS2 | 28-33 | 274 | 0.22 | 0.11 |
| CAS6 POS3 | 57-65 | 275 | 52.20 | 19.94 |
| CAS6 POS4 | 57-65 | 276 | 58.52 | 18.35 |
| CAS6 POS5 | 59-61 | 277 | 60.19 | 13.93 |
| CAS6 POS6 | 61-63 | 278 | 58.60 | 14.29 |
| CAS6 POS7 | 86-89 | 279 | 57.24 | 18.18 |
| CAS6 POS8 | 101-105 | 280 | 8.38 | 3.11 |
| CAS6 POS9 | 127-130 | 281 | 58.60 | 2.79 |
| CAS6 POS10 | 151-154 | 282 | 66.86 | 1.60 |
| CAS6 POS11 | 157-159 | 283 | 51.11 | 2.03 |
| CAS6 POS12 | Between | 284 | 31.68 | 4.60 |
| | 170-171 | | | |

In a third study, the efficiency of targeted mutagenesis of Cas-alpha 10 endonuclease variants, SEQ ID NOs:14, 48, 60, and 157, were compared when tested with two sgRNA recognition domains, SEQ ID NOs:61 and 327, at two target sites, MS26 and D8. Experiments were carried-out in Zea mays using Agrobacterium (Agro) transformation as described in Example 2. Two temperature regimens were performed. The first subjected Agro transformed embryos (once per day) to 45° C. for 4 hrs on days 3, 4, and 5 (3×4 hr 45° C.) after infection and the second to a continuous incubation at 37° C. on days 1-3 (1-3d 37° C.) following infection. For both regimens, embryos were placed at 28° C. before and after temperature treatment. In initial tests, immature embryos were harvested seven days after inoculation with Agro (OD₅₅₀ of 0.4) and the percent of targeted mutation assayed as described in Example 3.

Cas-alpha 10 endonuclease SEQ ID NO:48 in combination with sgRNA recognition domain SEQ ID NO:327 showed the highest frequency of targeted mutagenesis at both target sites and under both temperature treatments (Table 11). This resulted in a 11.50- and 12.61-fold improvement over the wildtype nuclease (SEQ ID NO:157) in combination with the sgRNA recognition domain SEQ ID NO:61 using the 45° C. temperature treatment (3×4 hr 45° C.) at the MS26 and D8 targets, respectively. Under the 37° C. regimen (1-3d 37° C.), a 1053.00- and 13.15-fold enhancement was observed at the MS26 and D8 sites, respectively.

TABLE 11

| Cas-alpha | Corresponding | | | | |
|---|---|---|---|---|---|
| Nuclease | sgRNA | Temper- | | % Targeted | Fold |
| (SEQ ID | (SEQ | ature | Target | Mutagenesis | Improve- |
| NO) | ID NO: ) | Regimen | Site | (n = 3) | ment |
| 157 | 352 | 3 × 4 hr 4 | MS26 | 1.35 | — |
| 14 | | 5° C. | | 5.24 | 3.88 |
| 48 | | | | 6.62 | 4.90 |
| 60 | | | | 4.79 | 3.55 |
| 157 | 354 | | | 4.67 | 3.46 |
| 14 | | | | 9.81 | 7.27 |
| 48 | | | | 15.53 | 11.50 |
| 60 | | | | 9.67 | 7.16 |
| 157 | 352 | 1-3 d | | 0.01 | — |
| 14 | | 37° C. | | 0.67 | 67.00 |
| 48 | | | | 1.66 | 166.00 |
| 60 | | | | 0.55 | 55.00 |
| 157 | 354 | | | 0.16 | 16.00 |
| 14 | | | | 5.66 | 566.00 |
| 48 | | | | 10.53 | 1053.00 |
| 60 | | | | 4.07 | 407.00 |
| 157 | 353 | 3 × 4 hr | D8 | 0.38 | — |
| 14 | | 45° C. | | 2.24 | 5.89 |
| 48 | | | | 2.73 | 7.18 |
| 60 | | | | 1.61 | 4.24 |
| 157 | 355 | | | 1.10 | 2.89 |
| 14 | | | | 2.76 | 7.26 |
| 48 | | | | 4.79 | 12.61 |
| 60 | | | | 2.12 | 5.58 |
| 157 | 353 | 1-3 d | | 0.13 | — |
| 14 | | 37° C. | | 0.30 | 2.31 |
| 48 | | | | 0.32 | 2.46 |
| 60 | | | | 0.22 | 1.69 |
| 157 | 355 | | | 0.16 | 1.23 |
| 14 | | | | 0.56 | 4.31 |
| 48 | | | | 1.71 | 13.15 |
| 60 | | | | 0.74 | 5.69 |

To confirm the findings in Table 11, T0 plants were regenerated for several of the experiments, and the percentage of plants with a predicted germline mutation calculated as described in Example 3. Results corroborated that Cas-alpha 10 endonuclease variant SEQ ID NO:48 when paired with the sgRNA recognition domain SEQ ID NO:327 produced the highest frequency of targeted mutagenesis out of the combinations initially tested (Table 12).

TABLE 12

| | | | | % of Plants | |
|---|---|---|---|---|---|
| | | | | with a | |
| Cas-alpha | | | | Predicted | |
| Nuclease | Corresponding | Temper- | | Targeted | No. of |
| (SEQ ID | sgRNA (SEQ | ature | Target | Germline | Plants |
| NO) | ID NO: ) | Regimen | Site | Mutation | Analyzed |
| 14 | 352 | 3 × 4 hr | MS26 | 29 | 190 |
| 14 | 354 | 45° C. | | 66 | 184 |
| 48 | | | | 77 | 169 |
| 60 | | | | 46 | 180 |
| 14 | 352 | 1-3 d | | 2 | 166 |
| 14 | 354 | 37° C. | | 47 | 114 |
| 48 | | | | 71 | 199 |
| 60 | | | | 42 | 222 |
| 14 | 353 | 3 × 4 hr | D8 | 37 | 164 |
| 14 | 355 | 45° C. | | 44 | 214 |
| 48 | | | | 63 | 143 |
| 60 | | | | 31 | 187 |
| 14 | 353 | 1-3 d | | 1 | 201 |
| 14 | 355 | 37° C. | | 6 | 164 |
| 48 | | | | 34 | 146 |
| 0 | | | | 15 | 177 |

1. An engineered Cas polypeptide comprising a sequence having 90% amino acid sequence identity to SEQ ID NO:14 and one or more of the following amino acids at positions relative to an alignment with SEQ ID NO:14: Glutamate at 34, Alanine at 36, Serine at 45, Aspartate at 54, Serine at 77, Glutamate at 81, or Glutamine at 85, wherein the engineered Cas polypeptide is capable of site specifically binding a target site of a polynucleotide.

2. An engineered Cas polypeptide, comprising:
(a) a sequence having 90% amino acid sequence identity to SEQ ID NO:14 a C-terminal tri-split RuvC domain and three zinc finger motifs; and
(b) one or more of the following amino acids at positions relative to an alignment with SEQ ID NO:14: Glutamate at 34, Alanine at 36, Serine at 45, Aspartate at 54, Serine at 77, Glutamate at 81, or Glutamine at 85;
wherein the engineered Cas polypeptide does not comprise at least one of the following amino acids at positions relative to an alignment with SEQ ID NO:14: Phenylalanine at position 38, Alanine at position 40, Histidine at position 79, Alanine at relative position 87, Threonine at relative position 335, Cysteine at position 409, Glutamate at position 421, Lysine at position 467, or Glutamate at position 468 and wherein the engineered Cas polypeptide is capable of site specifically binding a target site of a polynucleotide.

3. The engineered Cas polypeptide of claim 1, wherein the polypeptide has at least 95% amino acid sequence identity to a sequence selected from the group consisting of: SEQID NOs:14-56 or the group consisting of SEQID Nos:59 and 60, or the group consisting of SEQID NOs:309-323 and SEQID NO:362.

4. The engineered Cas polypeptide of claim 1, further comprising at least one of the following amino acids at positions relative to an alignment with SEQ ID NO:14: Aspartate or Glutamate at position 38, Aspartate at position 79, Proline at position 120, Aspartate at position 149, Glycine at 226, Glycine at 230, Histidine at position 293, Serine at position 298, Phenylalanine at position 306, Glutamate at 329, Serine at position 313, Asparagine at position 325, Glutamate at position 327, Valine at position 338, Cysteine at 376, Cysteine at 379, Cysteine at 395, Cysteine at 398, Cysteine at position 406, Lysine or Arginine at position 409, Asparagine or Arginine at position 421, Proline at position 430, Arginine at position 467, or Proline at position 468.

5. The engineered Cas polypeptide of claim 1, wherein the engineered Cas polypeptide is an endonuclease that has greater activity than SEQ ID NO: 14 at about 30 degrees Celsius.

6-10. (canceled)

11. The engineered Cas polypeptide of any one of claim 1, further comprising a guide polynucleotide comprising a variable targeting domain that comprises a region of complementarity to the target site of a polynucleotide.

12. The engineered Cas polypeptide of claim 11, wherein the guide polynucleotide variable targeting domain comprises fewer than 20 nucleotides.

13. (canceled)

14. The engineered Cas polypeptide of claim 1, wherein the engineered Cas polypeptide is an endonuclease that cleaves a double-stranded DNA polynucleotide.

15. The engineered Cas polypeptide of claim 1, wherein the engineered Cas polypeptide is catalytically inactive for endonuclease activity.

16. The engineered Cas polypeptide of claim 1, wherein the engineered Cas polypeptide recognizes a PAM sequence that comprises N(T>W>C)TTC.

17. (canceled)

18. The engineered Cas polypeptide of claim 1, wherein the engineered Cas polypeptide is part of a fusion protein, wherein the fusion protein further comprises a heterologous nuclease domain.

19. The engineered Cas polypeptide of claim 1, further comprising a deaminase.

20. A synthetic composition comprising the engineered Cas polypeptide of claim 1, further comprising a heterologous polynucleotide.

21. (canceled)

22. (canceled)

23. A synthetic composition comprising:
(a) an engineered Cas polypeptide in accordance with any claim 1;
(b) a target double-stranded DNA polynucleotide; and
(c) a guide polynucleotide comprising a variable targeting domain that comprises a region of complementarity to a target double-stranded DNA polynucleotide;
wherein the Cas polypeptide recognizes a PAM sequence on the target double-stranded DNA polynucleotide, wherein the guide polynucleotide and the Cas polypeptide form a complex that binds the target double-stranded DNA polynucleotide.

24. A polynucleotide encoding the engineered Cas polypeptide of claim 1.

25. (canceled)

26. (canceled)

27. The engineered Cas polypeptide of claim 1, wherein the engineered Cas polypeptide is attached to a solid matrix or the Cas polypeptide is complexed with a guide polynucleotide and the Cas polypeptide/guide polynucleotide complex is attached to a solid matrix.

28. A eukaryotic cell comprising the engineered Cas polypeptide of claim 1.

29-31. (canceled)

32. The eukaryotic cell of claim 28, wherein the eukaryotic cell is at a temperature of about 30 degrees Celsius or less.

33. A method of introducing a targeted edit in a target polynucleotide, the method comprising:
(a) providing the engineered Cas polypeptide and guide polynucleotide of claim 1, wherein the Cas polypeptide/guide polynucleotide form a complex that recognizes a PAM sequence on the target polynucleotide; and
(b) contacting the Cas polypeptide/guide polynucleotide complex with the target; and
(c) introducing a targeted edit in the target polynucleotide.

34. The method of claim 33, wherein the target polynucleotide is a target genomic sequence of a cell and the method comprises:
(i) delivering the Cas polypeptide/guide polynucleotide complex to the cell;
(ii) incubating the cell at a temperature of about 37 degrees or less, about 35 degrees Celsius or less, about 30 degrees Celsius or less, about 25 degrees Celsius or less, or about 20 degrees or less;
(iii) modifying at least one nucleotide in the target genomic sequence of the cell to generate a modified genomic sequence as compared to the target genomic sequence of the cell prior to the delivering the Cas polypeptide/guide polynucleotide complex; and
(iv) generating a whole organism from the cell, wherein the organism comprises the modified genomic sequence.

35. The method of claim 34, wherein the cell is a eukaryotic cell.

36-43. (canceled)

44. A kit comprising the engineered Cas polypeptide and solid matrix of claim 27.

45. A method of altering a target Cas polypeptide; the method comprising modifying the target Cas polypeptide to include one or more amino acid changes, such that when aligned with SEQ ID NO:14 and relative to the amino acid position numbers of SEQ ID NO:14, the modified Cas polypeptide comprises one or more of the following amino acids: Glutamate at position 34, Alanine at position 36, Serine at position 45, Aspartate at position 54, Serine at position 77, Glutamate at 81, or Glutamine at position 85, wherein the modified Cas polypeptide is capable of site specifically binding a target site of a polynucleotide.

46. The method of claim 45, wherein the method further comprises modifying the Cas polypeptide, such that when aligned with SEQ ID NO:14 and relative to the amino acid position numbers of SEQ ID NO:14, the modified Cas polypeptide further comprises: Aspartate or Glutamate at position 38, Aspartate at position 79, Proline at position 120, Aspartate at position 149, Glycine at 226, Glycine at 230, Histidine at position 293, Serine at position 298, Phenylalanine at position 306, Glutamate at 329, Serine at position 313, Asparagine at position 325, Glutamate at position 327, Valine at position 338, Cysteine at 376, Cysteine at 379, Cysteine at 395, Cysteine at 398, Cysteine at position 406, Lysine or Arginine at position 409, Asparagine or Arginine at position 421, Proline at position 430, Arginine at position 467, or Proline at position 468.

47. (canceled)

48. A system comprising a Cas polypeptide and an engineered guide RNA (egRNA), wherein the egRNA is based on a template guide RNA sequence (sgRNA) that comprises a Cas polypeptide recognition domain and wherein the egRNA comprises (i) a deletion, substitution or an insertion at one or more of nucleotide positions 1-3, 3-5, 5-7, 7-9, 59-61, 61-63, 63-65, 147-149, 149-151, 151-153, 153-155, 157-159, 159-161, 163-165, or 167-169 of the sgRNA Cas polypeptide recognition domain or (ii) one of SEQ ID NOs: 62-156, SEQ ID NOs: 192-207, SEQ ID NOs: 260-284, SEQ ID NOs: 324-327, SEQ ID NOs: 346-355, or SEQ ID NO:362.

49-55. (canceled)
